SEQUENCE LISTING (243 sequences)

SEQ ID NO: 1 - protein, 16 aa, Measles virus
  PEPTIDE (1)..(16): MvF Th
DLSDLKGLLLHKLDGL

SEQ ID NO: 2 - protein, 16 aa, Measles virus
  PEPTIDE (1)..(16): MvF Th
EISDEIRLIIIKRIEI

SEQ ID NO: 3 - protein, 16 aa, Measles virus
  PEPTIDE (1)..(16): MvF Th
DVSDVKGVVVHKVDGV

SEQ ID NO: 4 - protein, 16 aa, Measles virus
  PEPTIDE (1)..(16): MvF Th
DFSDFKGFFFHKFDGF

SEQ ID NO: 5 - protein, 16 aa, Measles virus
  PEPTIDE (1)..(16): MvF Th
  SITE (1)..(1): D or E
  SITE (2)..(2): L or I or V or F
  SITE (4)..(4): D or E
  SITE (5)..(5): L or I or V or F
  SITE (6)..(6): K or R
  SITE (8)..(8): L or I or V or F
  SITE (9)..(9): L or I or V or F
  SITE (10)..(10): L or I or V or F
  SITE (12)..(12): K or R
  SITE (13)..(13): L or I or V or F
  SITE (14)..(14): D or E
  SITE (16)..(16): L or I or V or F
XXSXXXGXXXHXXXGX

SEQ ID NO: 6 - protein, 15 aa, Measles virus
  PEPTIDE (1)..(15): MvF1 Th
LSEIKGVIVHRLEGV

SEQ ID NO: 7 - protein, 15 aa, Measles virus
  PEPTIDE (1)..(15): MvF 2 Th
ISEIKGVIVHKIEGI

SEQ ID NO: 8 - protein, 19 aa, Measles virus
  PEPTIDE (1)..(19): MvF3 Th
ISISEIKGVIVHKIEGILF

SEQ ID NO: 9 - protein, 19 aa, Measles virus
  PEPTIDE (1)..(19): MvF 3Th
ISITEIRTVIVTRIETILF

SEQ ID NO: 10 - protein, 19 aa, Measles virus
  PEPTIDE (1)..(19): MvF3 Th
  SITE (4)..(4): S or T
  SITE (7)..(7): K or R
  SITE (8)..(8): G or T
  SITE (12)..(12): H or T
  SITE (13)..(13): K or R
  SITE (16)..(16): G or T
ISIXEIXXVIVXXIETILF

SEQ ID NO: 11 - protein, 22 aa, Measles virus
  PEPTIDE (1)..(22): KKKMvF3 Th
KKKISISEIKGVIVHKIEGILF

SEQ ID NO: 12 - protein, 22 aa, Measles virus
  PEPTIDE (1)..(22): KKKMvF3 Th
KKKISITEIRTVIVTRIETILF

SEQ ID NO: 13 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 14 - protein, 19 aa, Measles virus
  PEPTIDE (1)..(19): MvF4 Th
ISISEIKGVIVHKIETILF

SEQ ID NO: 15 - protein, 19 aa, Measles virus
  PEPTIDE (1)..(19): MvF4 Th
ISITEIRTVIVTRIETILF

SEQ ID NO: 16 - protein, 19 aa, Measles virus
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 17 - protein, 19 aa, Measles virus
  PEPTIDE (1)..(19): MvF5 Th (UBITh1)
ISITEIKGVIVHRIETILF

SEQ ID NO: 18 - protein, 22 aa, Measles virus
  PEPTIDE (1)..(22): KKKMvF5 Th (UBITh1a)
KKKISITEIKGVIVHRIETILF

SEQ ID NO: 19 - protein, 18 aa, Hepatitis B virus
  PEPTIDE (1)..(18): HBsAg1 Th
KKKLFLLTKLLTLPQSLD

SEQ ID NO: 20 - protein, 18 aa, Hepatitis B virus
  PEPTIDE (1)..(18): HBsAg1 Th
RRRIKIITRIITIPLSIR

SEQ ID NO: 21 - protein, 18 aa, Hepatitis B virus
  PEPTIDE (1)..(18): HBsAg1 Th
KKKVRVVTKVVTVPISVD

SEQ ID NO: 22 - protein, 18 aa, Hepatitis B virus
  PEPTIDE (1)..(18): HBsAg1 Th
KKKFFFFTKFFTFPVSFD

SEQ ID NO: 23 - protein, 18 aa, Hepatitis B virus
  PEPTIDE (1)..(18): HBsAg1 Th
KKKLFLLTKLLTLPFSLD

SEQ ID NO: 24 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 25 - protein, 18 aa, Hepatitis B virus
  PEPTIDE (1)..(18): HBsAg2 Th
KKKIITITRIITIPQSLD

SEQ ID NO: 26 - protein, 18 aa, Hepatitis B virus
  PEPTIDE (1)..(18): HBsAg2 Th
KKKFFLLTRILTIITTID

SEQ ID NO: 27 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 28 - protein, 18 aa, Hepatitis B virus
  PEPTIDE (1)..(18): HBsAg 3 Th
KKKIITITRIITIITTID

SEQ ID NO: 29 - protein, 15 aa, Hepatitis B virus
  PEPTIDE (1)..(15): HBsAg4 Th (UBITh4)
FFLLTRILTIPQSLD

SEQ ID NO: 30 - protein, 18 aa, Hepatitis B virus
  PEPTIDE (1)..(18): KKK-HBsAg Th
KKKFFLLTRILTIPQSLD

SEQ ID NO: 31 - protein, 14 aa, Hepatitis B virus
  PEPTIDE (1)..(14): HBsAg Th
FFLLTRILTIPQSL

SEQ ID NO: 32 - protein, 24 aa, Bordetella pertussis
  PEPTIDE (1)..(24): Bordetella pertussis Th
GAYARCPNGTRALTVAELRGNAEL

SEQ ID NO: 33 - protein, 25 aa, Vibrio cholerae
  PEPTIDE (1)..(25): Cholera Toxin Th
ALNIWDRFDVFCTLGATTGYLKGNS

SEQ ID NO: 34 - protein, 15 aa, Clostridium tetani
  PEPTIDE (1)..(15): Clostridium tetani TT1 Th
QYIKANSKFIGITEL

SEQ ID NO: 35 - protein, 17 aa, Clostridium tetani
  PEPTIDE (1)..(17): Clostridium tetani 1 Th
KKQYIKANSKFIGITEL

SEQ ID NO: 36 - protein, 21 aa, Clostridium tetani
  PEPTIDE (1)..(21): Clostridium tetani  TT2 Th
FNNFTVSFWLRVPKVSASHLE

SEQ ID NO: 37 - protein, 16 aa, Clostridium tetani
  PEPTIDE (1)..(16): Clostridium tetani TT3 Th
KFIIKRYTPNNEIDSF

SEQ ID NO: 38 - protein, 16 aa, Clostridium tetani
  PEPTIDE (1)..(16): Clostridium tetani  TT4 Th
VSIDKFRIFCKALNPK

SEQ ID NO: 39 - protein, 17 aa, Clostridium tetani
  PEPTIDE (1)..(17): Clostridium tetani 2 Th
WVRDIIDDFTNESSQKT

SEQ ID NO: 40 - protein, 23 aa, diphtheria bacilli
  PEPTIDE (1)..(23): Diphtheria Th
DSETADNLEKTVAALSILPGHGC

SEQ ID NO: 41 - protein, 19 aa, Epstein-Barr virus
  PEPTIDE (1)..(19): EBV BHRF1 Th
AGLTLSLLVICSYLFISRG

SEQ ID NO: 42 - protein, 20 aa, Epstein-Barr virus
  PEPTIDE (1)..(20): EBV EBNA-1 Th
PGPLRESIVCYFMVFLQTHI

SEQ ID NO: 43 - protein, 18 aa, Epstein-Barr virus
  PEPTIDE (1)..(18): EBV CP Th
VPGLYSPCRAFFNKEELL

SEQ ID NO: 44 - protein, 15 aa, Epstein-Barr virus
  PEPTIDE (1)..(15): EBV GP340 Th
TGHGARTSTEPTTDY

SEQ ID NO: 45 - protein, 13 aa, Epstein-Barr virus
  PEPTIDE (1)..(13): EBV BPLF1 Th
KELKRQYEKKLRQ

SEQ ID NO: 46 - protein, 11 aa, Epstein-Barr virus
  PEPTIDE (1)..(11): EBV EBNA-2 Th
TVFYNIPPMPL

SEQ ID NO: 47 - protein, 14 aa, Human cytomegalovirus
  PEPTIDE (1)..(14): HCMV IE1 Th
DKREMWMACIKELH

SEQ ID NO: 48 - protein, 11 aa, Influenza virus
  PEPTIDE (1)..(11): Influenza Matrix protein 1_1 Th
FVFTLTVPSER

SEQ ID NO: 49 - protein, 15 aa, Influenza virus
  PEPTIDE (1)..(15): Influenza Matrix protein 1_2 Th
SGPLKAEIAQRLEDV

SEQ ID NO: 50 - protein, 9 aa, Influenza virus
  PEPTIDE (1)..(9): Influenza Non-structural protein 1 Th
DRLRRDQKS

SEQ ID NO: 51 - protein, 21 aa, Plasmodium falciparum
  PEPTIDE (1)..(21): Plasmodium falciparum Th
DHEKKHAKMEKASSVFNVVNS

SEQ ID NO: 52 - protein, 17 aa, Schistosoma mansoni
  PEPTIDE (1)..(17): Schistosoma mansoni Th
KWFKTNAPNGVDEKHRH

SEQ ID NO: 53 - protein, 4 aa, Artificial Sequence
  Synthetic peptide
  SITE (1)..(1): epsilon-K
  PEPTIDE (1)..(4): epsilon-K-KKK as a spacer
KKKK

SEQ ID NO: 54 - protein, 4 aa, Artificial Sequence
  Synthetic peptide
  PEPTIDE (1)..(4): KKK-epsilon-K as a spacer
  SITE (4)..(4): epsilon-K
KKKK

SEQ ID NO: 55 - protein, 6 aa, Artificial Sequence
  Synthetic peptide
  PEPTIDE (1)..(6): Flexible Hinge Spacer
  SITE (3): Xaa is any amino acid
  SITE (5): Xaa is any amino acid
PPXPXP

SEQ ID NO: 56 - protein, 14 aa, Homo sapiens
  PEPTIDE (1)..(14): Abeta1-14
DAEFRHDSGYEVHH

SEQ ID NO: 57 - protein, 16 aa, Homo sapiens
  PEPTIDE (1)..(16): Abeta1-16
DAEFRHDSGYEVHHQK

SEQ ID NO: 58 - protein, 26 aa, Homo sapiens
  PEPTIDE (1)..(26): Abeta17-42
LVFFAEDVGSNKGAIIGLMVGGVVIA

SEQ ID NO: 59 - protein, 28 aa, Homo sapiens
  PEPTIDE (1)..(28): Abeta1-28
DAEFRHDSGYEVHHQKLVFFAEDVGSNK

SEQ ID NO: 60 - protein, 42 aa, Homo sapiens [sequence omitted]

SEQ ID NO: 61 - protein, 22 aa, Homo sapiens
  PEPTIDE (1)..(22): alpha-Syn111-132 (Derived from GenBank NP_000336)
GILEDMPVDPDNEAYEMPSEEG

SEQ ID NO: 62 - protein, 39 aa, Homo sapiens
  PEPTIDE (1)..(39): IgE-EMPD1-39
GLAGGSAQSQRAPDRVLCHSGQQQGLPRAAGGSVPHPRC

SEQ ID NO: 63 - protein, 30 aa, Homo sapiens
  PEPTIDE (1)..(30): Tau379-408 (Derived from GenBank AGF19246.1)
RENAKAKTDHGAEIVYKSPVVSGDTSPRHL

SEQ ID NO: 64 - protein, 48 aa, Homo sapiens
  PEPTIDE (1)..(48): IL-3197-144 (Derived from Uniprot C7G0W1-1; GenBank BAH97742.1)
LSDKNIIDKIIEQLDKLKFQHEPETEISVPADTFECKSFILTILQQFS

SEQ ID NO: 65 - protein, 37 aa, Artificial Sequence
  Synthetic peptide
  PEPTIDE (1)..(14): Abeta 1-14
  PEPTIDE (15)..(18): epsilon-K-KKK as a spacer
  SITE (15)..(15): epsilon-K
  PEPTIDE (19)..(37): MvF 4 Th
  SITE (22)..(22): S or T
  SITE (25)..(25): K or R
  SITE (26)..(26): G or T
  SITE (30)..(30): H or T
  SITE (31)..(31): K or R
DAEFRHDSGYEVHHKKKKISIXEIXXVIVXXIETILF

SEQ ID NO: 66 - protein, 33 aa, Artificial Sequence
  Synthetic peptide
  PEPTIDE (1)..(14): Abeta 1-14
  SITE (15)..(15): epsilon-K
  PEPTIDE (16)..(33): HBsAg 2 Th
  SITE (19)..(19): I or F
  SITE (20)..(20): I or F
  SITE (21)..(21): T or L
  SITE (22)..(22): I or L
  SITE (26)..(26): I or L
  SITE (29)..(29): P or I
  SITE (30)..(30): Q or T
  SITE (31)..(31): S or T
  SITE (32)..(32): L or I
DAEFRHDSGYEVHHKKKKXXXXTRIXTIXXXXD

SEQ ID NO: 67 - protein, 37 aa, Artificial Sequence
  Synthetic peptide
  PEPTIDE (1)..(14): Abeta 1-14
  SITE (15)..(15): epsilon K
  PEPTIDE (15)..(18): epsilon K-KKK as a spacer
  PEPTIDE (19)..(37): MvF5 Th (UBITh1)
DAEFRHDSGYEVHHKKKKISITEIKGVIVHRIETILF

SEQ ID NO: 68 - protein, 33 aa, Artificial Sequence
  Synthetic peptide
  PEPTIDE (1)..(14): Abeta 1-14
  SITE (15)..(15): epsilon K
  PEPTIDE (16)..(33): HBsAg3 Th
DAEFRHDSGYEVHHKKKKIITITRIITIITTID

SEQ ID NO: 69 - protein, 16 aa, Homo sapiens
  PEPTIDE (1)..(16): Tau145-160
ADGKTKIATPRGAAPP

SEQ ID NO: 70 - protein, 50 aa, Homo sapiens
  PEPTIDE (1)..(50): Tau1-50
MAEPRQEFEVMEDHAGTYGLGDRKDQGGYTMHQDQEGDTDAGLKESPLQT

SEQ ID NO: 71 - protein, 15 aa, Homo sapiens
  PEPTIDE (1)..(15): Tau297-311
IKHVPGGGSVQIVYK

SEQ ID NO: 72 - protein, 31 aa, Homo sapiens
  PEPTIDE (1)..(31): IL-31 85-115 (S85 to C85, I115 to C115)
CPAIRAYLKTIRQLDNKSVIDEIIEHLDKLC

SEQ ID NO: 73 - protein, 25 aa, Melanomma radicans
  PEPTIDE (1)..(25): Melanoma Neoantigen
SGSPPLRVSVGDFSQEFSPQEAQQD

SEQ ID NO: 74 - protein, 10 aa, Artificial Sequence
  Glioma
  PEPTIDE (1)..(10): Histone 3 Variant H3.3K27M for Glioma
RMSAPSTGGV

SEQ ID NO: 75 - protein, 24 aa, Artificial Sequence
  KRAS Metastatic Colorectal Cancer
  PEPTIDE (1)..(24): KRAS  Metastatic Colorectal Cancer (MUTANT G-to-D)
MTEYKLVVVGADGVGKSALTIQLI

SEQ ID NO: 76 - protein, 32 aa, Human immunodeficiency virus
  PEPTIDE (1)..(32): HIV CTL Gag P24
SILDIKQGPKEPFRDYVDRFYKTLRAEQASQE

SEQ ID NO: 77 - protein, 22 aa, Human immunodeficiency virus
  PEPTIDE (1)..(22): HIV CTL Gag P17
ASRELERFAVNPGLLETSEGCR

SEQ ID NO: 78 - protein, 20 aa, Human immunodeficiency virus
  PEPTIDE (1)..(20): HIV CTL Gag 293-312
FRDYVDRFYKTLRAEQASQE

SEQ ID NO: 79 - protein, 35 aa, Human immunodeficiency virus
  PEPTIDE (1)..(35): HIV CTL POL PRT
PRTKMIGGIGGFIKVRQYDQILIECGHKAIGTVLV

SEQ ID NO: 80 - protein, 26 aa, Human immunodeficiency virus
  PEPTIDE (1)..(26): HIV CTL POL RT
LRWGFTTPDKKHQKEPPFLWMGYELH

SEQ ID NO: 81 - protein, 17 aa, Human immunodeficiency virus
  PEPTIDE (1)..(17): HIV CTL POL INT
TKELQKQITKIQNFRVY

SEQ ID NO: 82 - protein, 16 aa, Human immunodeficiency virus
  PEPTIDE (1)..(16): HIV CTL VIF
CFADSAIRKAILGHIV

SEQ ID NO: 83 - protein, 9 aa, human herpesvirus 1
  PEPTIDE (1)..(9): HSV CTL VP11/12 (66-74)
FLTCTDRSV

SEQ ID NO: 84 - protein, 9 aa, human herpesvirus 1
  PEPTIDE (1)..(9): HSV CTL VP11/12 (197-205)
RIQQYMFFM

SEQ ID NO: 85 - protein, 9 aa, human herpesvirus 1
  PEPTIDE (1)..(9): HSV CTL VP11/12 (220-228)
RLNELLAYV

SEQ ID NO: 86 - protein, 17 aa, human herpesvirus 1
  PEPTIDE (1)..(17): HSV CTL VP11/12 (230-246)
VLYRWASWMLWTTDKHV

SEQ ID NO: 87 - protein, 9 aa, human herpesvirus 1
  PEPTIDE (1)..(9): HSV CTL VP11/12 (702-710)
ALSALLTKL

SEQ ID NO: 88 - protein, 8 aa, human herpesvirus 1
  PEPTIDE (1)..(8): HSV CTL gamma1 gB498
SSIEFARL

SEQ ID NO: 89 - protein, 9 aa, human herpesvirus 1
  PEPTIDE (1)..(9): HSV CTL gamma1 gB452
YQPLLSNTL

SEQ ID NO: 90 - protein, 9 aa, human herpesvirus 1
  PEPTIDE (1)..(9): HSV CTL gamma1 gB560
SARMLGDVM

SEQ ID NO: 91 - protein, 9 aa, human herpesvirus 1
  PEPTIDE (1)..(9): HSV CTL gamma1 gG4
GAMRAVVPI

SEQ ID NO: 92 - protein, 8 aa, human herpesvirus 1
  PEPTIDE (1)..(8): HSV CTL beta PR1982
FAPLFTNL

SEQ ID NO: 93 - protein, 8 aa, human herpesvirus 1
  PEPTIDE (1)..(8): HSV CTL beta PR1_822
QTFDFGRL

SEQ ID NO: 94 - protein, 9 aa, human herpesvirus 1
  PEPTIDE (1)..(9): HSV CTL beta PR1_372
FGLLNYALV

SEQ ID NO: 95 - protein, 8 aa, human herpesvirus 1
  PEPTIDE (1)..(8): HSV CTL beta ICP_876
GAINFINL

SEQ ID NO: 96 - protein, 11 aa, human herpesvirus 1
  PEPTIDE (1)..(11): HSV CTL beta ICP_168
AVCINNTFLHL

SEQ ID NO: 97 - protein, 10 aa, human herpesvirus 1
  PEPTIDE (1)..(10): HSV CTL beta PR2_81
SFYRFLFAFL

SEQ ID NO: 98 - protein, 9 aa, human herpesvirus 1
  PEPTIDE (1)..(9): HSV CTL beta PR2_279
AAIENYVRF

SEQ ID NO: 99 - protein, 9 aa, human herpesvirus 1
  PEPTIDE (1)..(9): HSV CTL beta UL9_251
FLPRLGTEL

SEQ ID NO: 100 - protein, 8 aa, human herpesvirus 1
  PEPTIDE (1)..(8): HSV CTL beta UL41_181
LGYAYINS

SEQ ID NO: 101 - protein, 9 aa, human herpesvirus 1
  PEPTIDE (1)..(9): HSV CTL gamma2 gC8
LAVVLWSLL

SEQ ID NO: 102 - protein, 9 aa, human herpesvirus 1
  PEPTIDE (1)..(9): HSV CTL beta UL28_629
YSVENVGLL

SEQ ID NO: 103 - protein, 9 aa, human herpesvirus 1
  PEPTIDE (1)..(9): HSV CTL gamma2 gH391
FAFVNAAHA

SEQ ID NO: 104 - protein, 9 aa, human herpesvirus 1
  PEPTIDE (1)..(9): HSV CTL gamma2 gK54
WMKMNQTLL

SEQ ID NO: 105 - protein, 20 aa, human herpesvirus 1
  PEPTIDE (1)..(20): HSV CTL gB161-180
ATMYYKDVTVSQVWFGHRYS

SEQ ID NO: 106 - protein, 15 aa, human herpesvirus 1
  PEPTIDE (1)..(15): HSV CTL gB 666-680
TVSTFIDLNITMLED

SEQ ID NO: 107 - protein, 8 aa, Foot-and-mouth disease virus
  PEPTIDE (1)..(8): FMDV CTL L_65
EPFFDWVY

SEQ ID NO: 108 - protein, 9 aa, Foot-and-mouth disease virus
  PEPTIDE (1)..(9): FMDV CTL VP4_227
YMQQYQNSM

SEQ ID NO: 109 - protein, 9 aa, Foot-and-mouth disease virus
  PEPTIDE (1)..(9): FMDV CTL VP2_314
SSVGVTYGY

SEQ ID NO: 110 - protein, 8 aa, Foot-and-mouth disease virus
  PEPTIDE (1)..(8): FMDV CTL VP2_346
RFFKTHLF

SEQ ID NO: 111 - protein, 11 aa, Foot-and-mouth disease virus
  PEPTIDE (1)..(11): FMDV CTL VP2_385
AYMRNGWDVEV

SEQ ID NO: 112 - protein, 13 aa, Foot-and-mouth disease virus
  PEPTIDE (1)..(13): FMDV CTL VP2_421
RELYQLTLFPHQF

SEQ ID NO: 113 - protein, 8 aa, Foot-and-mouth disease virus
  PEPTIDE (1)..(8): FMDV CTL VP3_622
KARYMIAY

SEQ ID NO: 114 - protein, 8 aa, Foot-and-mouth disease virus
  PEPTIDE (1)..(8): FMDV CTL 2C1309
IIATTNLY

SEQ ID NO: 115 - protein, 11 aa, Foot-and-mouth disease virus
  PEPTIDE (1)..(11): FMDV CTL 2C1369
FQYDCALLNGM

SEQ ID NO: 116 - protein, 10 aa, Foot-and-mouth disease virus
  PEPTIDE (1)..(10): FMDV CTL 3C1720
MLSDAALMVL

SEQ ID NO: 117 - protein, 19 aa, Foot-and-mouth disease virus
  PEPTIDE (1)..(19): FMDV CTL 3D2083
WQRFGTHFAQYRNVWDVDY

SEQ ID NO: 118 - protein, 11 aa, Foot-and-mouth disease virus
  PEPTIDE (1)..(11): FMDV CTL 3D2159
NTILNNIYVLY

SEQ ID NO: 119 - protein, 31 aa, Foot-and-mouth disease virus
  PEPTIDE (1)..(31): FMDV CTL 3D2231/43/58
SITDVTFLKKKHMDYGTGFYKKKTLEAILSF

SEQ ID NO: 120 - protein, 17 aa, Foot-and-mouth disease virus
  PEPTIDE (1)..(17): FMDV CTL 3D2295
FEPFQGLFEIPSYRSLY

SEQ ID NO: 121 - protein, 10 aa, Foot-and-mouth disease virus
  PEPTIDE (1)..(10): FMDV CTL 3D2302
FEIPSYRSLY

SEQ ID NO: 122 - protein, 9 aa, Foot-and-mouth disease virus
  PEPTIDE (1)..(9): FMDV CTL Type O 750
RRQHTDVSF

SEQ ID NO: 123 - protein, 9 aa, Foot-and-mouth disease virus
  PEPTIDE (1)..(9): FMDV CTL Type O 881
RTLPTSFNY

SEQ ID NO: 124 - protein, 14 aa, Porcine reproductive and respiratory syndrome virus
  PEPTIDE (1)..(14): PRRSV CTL ORF1a
RLGKIISLCQVIED

SEQ ID NO: 125 - protein, 15 aa, Porcine reproductive and respiratory syndrome virus
  PEPTIDE (1)..(15): PRRSV CTL ORF1a
VPVITCGVVHLLAII

SEQ ID NO: 126 - protein, 9 aa, Porcine reproductive and respiratory syndrome virus
  PEPTIDE (1)..(9): PRRSV CTL ORF7
LSDSGRISY

SEQ ID NO: 127 - protein, 9 aa, Porcine reproductive and respiratory syndrome virus
  PEPTIDE (1)..(9): PRRSV CTL ORF2
RTAPNEIAF

SEQ ID NO: 128 - protein, 9 aa, Porcine reproductive and respiratory syndrome virus
  PEPTIDE (1)..(9): PRRSV CTL ORF2a
ASDWFAPRY

SEQ ID NO: 129 - protein, 10 aa, Porcine reproductive and respiratory syndrome virus
  PEPTIDE (1)..(10): PRRSV CTL ORF5
MSWRYSCTRY

SEQ ID NO: 130 - protein, 10 aa, Porcine reproductive and respiratory syndrome virus
  PEPTIDE (1)..(10): PRRSV CTL NSP9
TTMPSGFELY

SEQ ID NO: 131 - protein, 9 aa, Porcine reproductive and respiratory syndrome virus
  PEPTIDE (1)..(9): PRRSV CTL NSP10
NSFLDEAAY

SEQ ID NO: 132 - protein, 10 aa, Porcine reproductive and respiratory syndrome virus
  PEPTIDE (1)..(10): PRRSV CTL ORF6
RGRLLGLLHL

SEQ ID NO: 133 - protein, 9 aa, Porcine reproductive and respiratory syndrome virus
  PEPTIDE (1)..(9): PRRSV CTL ORF5
LYRWRSPVI

SEQ ID NO: 134 - protein, 10 aa, Porcine reproductive and respiratory syndrome virus
  PEPTIDE (1)..(10): PRRSV CTL NSP9
MPNYHWWVEH

SEQ ID NO: 135 - protein, 9 aa, Porcine reproductive and respiratory syndrome virus
  PEPTIDE (1)..(9): PRRSV CTL NSP9
EVALSAQII

SEQ ID NO: 136 - protein, 9 aa, Porcine reproductive and respiratory syndrome virus
  PEPTIDE (1)..(9): PRRSV CTL Type 1_4596
CLFAILLAT

SEQ ID NO: 137 - protein, 12 aa, Porcine reproductive and respiratory syndrome virus
  PEPTIDE (1)..(12): PRRSV CTL Type 1_4721
CAFAAFVCFVIR

SEQ ID NO: 138 - protein, 9 aa, Porcine reproductive and respiratory syndrome virus
  PEPTIDE (1)..(9): PRRSV CTL Type 1_5028
KPEKPHFPL

SEQ ID NO: 139 - protein, 9 aa, Porcine reproductive and respiratory syndrome virus
  PEPTIDE (1)..(9): PRRSV CTL Type 1_5083
FMLPVAHTV

SEQ ID NO: 140 - protein, 9 aa, Porcine reproductive and respiratory syndrome virus
  PEPTIDE (1)..(9): PRRSV CTL Type 2_2702
TMPPGFELY

SEQ ID NO: 141 - protein, 15 aa, Porcine reproductive and respiratory syndrome virus
  PEPTIDE (1)..(15): PRRSV CTL Type 2_4765
LAALICFVIRLAKNC

SEQ ID NO: 142 - protein, 16 aa, Porcine reproductive and respiratory syndrome virus
  PEPTIDE (1)..(16): PRRSV CTL Type 2_4797
KGRLYRWRSPVIIVEK

SEQ ID NO: 143 - protein, 15 aa, Artificial Sequence
  Classical swine fever virus
  PEPTIDE (1)..(15): CSFV CTL Type 1_1446
KHKVRNEVMVHWFDD

SEQ ID NO: 144 - protein, 9 aa, Artificial Sequence
  Classical swine fever virus
  PEPTIDE (1)..(9): CSFV CTL Type 1_2276
ENALLVALF

SEQ ID NO: 145 - protein, 11 aa, Homo sapiens
  PEPTIDE (1)..(11): IL-6 73-83
CFQSGFNEETC

SEQ ID NO: 146 - DNA, 32 bp, Artificial Sequence
  CpG1 oligonucleotide ODN
tcgtcgttttgtcgttttgtcgttttgtcgtt

SEQ ID NO: 147 - DNA, 24 bp, Artificial Sequence
  CpG2 oligonucleotide ODN
  misc_feature (1)..(1): phosphorothioate group
tcgtcgttttgtcgttttgtcgtt

SEQ ID NO: 148 - DNA, 24 bp, Artificial Sequence
  CpG3 Oligonucleotide ODN
tcgtcgttttgtcgttttgtcgtt

SEQ ID NO: 149 - protein, 45 aa, Artificial Sequence
  Alpha-Synuclein peptide immunogen construct
  PEPTIDE (1)..(19): MvF5 Th (UBITh1)
  SITE (20)..(20): epsilon-K
  PEPTIDE (20)..(23): epsilon-K-KKK spacer
  PEPTIDE (24)..(45): alpha-Synuclein (G111-G132)
ISITEIKGVIVHRIETILFKKKKGILEDMPVDPDNEAYEMPSEEG

SEQ ID NO: 150 - protein, 44 aa, Artificial Sequence
  Alpha-Synuclein peptide immunogen construct
  PEPTIDE (1)..(18): HBsAg3 Th (UBITh2)
  SITE (19)..(19): epsilon-K
  PEPTIDE (19)..(22): epsilon-K-KKK spacer
  PEPTIDE (23)..(44): alpha-Synuclein (G111-G132)
KKKIITITRIITIITTIDKKKKGILEDMPVDPDNEAYEMPSEEG

SEQ ID NO: 151 - protein, 45 aa, Artificial Sequence
  Alpha-Synuclein peptide immunogen construct
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon-K-KKK spacer
  PEPTIDE (24)..(45): alpha-Synuclein (G111-G132)
ISIXEIXXVIVXXIETILFKKKKGILEDMPVDPDNEAYEMPSEEG

SEQ ID NO: 152 - protein, 43 aa, Artificial Sequence
  Alpha-Synuclein peptide immunogen construct
  PEPTIDE (1)..(17): Clostridium tetani1 Th
  PEPTIDE (18)..(21): epsilon-K-KKK spacer
  SITE (18)..(18): epsilon-K
  PEPTIDE (22)..(43): alpha-Synuclein (G111-G132)
KKQYIKANSKFIGITELKKKKGILEDMPVDPDNEAYEMPSEEG

SEQ ID NO: 153 - protein, 41 aa, Artificial Sequence
  alpha-Synuclein (G111-G132)
  PEPTIDE (1)..(15): MvF1 Th
  PEPTIDE (16)..(19): epsilon-K-KKK spacer
  SITE (16)..(16): epsilon-K
  PEPTIDE (20)..(41): alpha-Synuclein (G111-G132)
LSEIKGVIVHRLEGVKKKKGILEDMPVDPDNEAYEMPSEEG

SEQ ID NO: 154 - protein, 50 aa, Artificial Sequence
  Synthetic peptide
  PEPTIDE (1)..(24): Bordetella pertussis Th
  PEPTIDE (25)..(28): epsilon-K-KKK spacer
  SITE (25)..(25): epsilon-K
  PEPTIDE (29)..(50): alpha-Synuclein (G111-G132)
GAYARCPNGTRALTVAELRGNAELKKKKGILEDMPVDPDNEAYEMPSEEG

SEQ ID NO: 155 - protein, 43 aa, Artificial Sequence
  Alpha-Synuclein peptide immunogen construct
  PEPTIDE (1)..(17): Clostridium tetani2 Th
  PEPTIDE (18)..(21): epsilon-K-KKK spacer
  SITE (18)..(18): epsilon-K
  PEPTIDE (22)..(43): alpha-Synuclein (G111-G132)
WVRDIIDDFTNESSQKTKKKKGILEDMPVDPDNEAYEMPSEEG

SEQ ID NO: 156 - protein, 49 aa, Artificial Sequence
  Alpha-Synuclein peptide immunogen construct
  PEPTIDE (1)..(23): Diphtheria Th
  PEPTIDE (24)..(27): epsilon-K-KKK spacer
  SITE (24)..(24): epsilon-K
  PEPTIDE (28)..(49): alpha-Synuclein (G111-G132)
DSETADNLEKTVAALSILPGHGCKKKKGILEDMPVDPDNEAYEMPSEEG

SEQ ID NO: 157 - protein, 47 aa, Artificial Sequence
  Alpha-Synuclein peptide immunogen construct
  PEPTIDE (1)..(21): Plasmodium falciparum Th
  PEPTIDE (22)..(25): epsilon-K-KKK spacer
  SITE (22)..(22): epsilon-K
  PEPTIDE (26)..(47): alpha-Synuclein (G111-G132)
DHEKKHAKMEKASSVFNVVNSKKKKGILEDMPVDPDNEAYEMPSEEG

SEQ ID NO: 158 - protein, 43 aa, Artificial Sequence
  Alpha-Synuclein peptide immunogen construct
  PEPTIDE (1)..(17): Schistosoma mansoni Th
  PEPTIDE (18)..(21): epsilon-K-KKK spacer
  SITE (18)..(18): epsilon-K
  PEPTIDE (22)..(43): alpha-Synuclein (G111-G132)
KWFKTNAPNGVDEKHRHKKKKGILEDMPVDPDNEAYEMPSEEG

SEQ ID NO: 159 - protein, 51 aa, Artificial Sequence
  Alpha-Synuclein peptide immunogen construct
  PEPTIDE (1)..(25): Cholera Toxin Th
  PEPTIDE (26)..(29): epsilon-K-KKK spacer
  SITE (26)..(26): epsilon-K
  PEPTIDE (30)..(51): alpha-Synuclein (G111-G132)
ALNIWDRFDVFCTLGATTGYLKGNSKKKKGILEDMPVDPDNEAYEMPSEEG

SEQ ID NO: 160 - protein, 41 aa, Artificial Sequence
  Alpha-Synuclein peptide immunogen construct
  PEPTIDE (1)..(15): MvF2 Th
  PEPTIDE (16)..(19): epsilon-K-KKK spacer
  SITE (16)..(16): epsilon-K
  PEPTIDE (20)..(41): alpha-Synuclein (G111-G132)
ISEIKGVIVHKIEGIKKKKGILEDMPVDPDNEAYEMPSEEG

SEQ ID NO: 161 - protein, 48 aa, Artificial Sequence
  Synthetic peptide
  PEPTIDE (1)..(22): KKKMvF3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
  SITE (23)..(23): epsilon K as a spacer
  PEPTIDE (23)..(26): epsilon-K-KKK spacer
  PEPTIDE (27)..(48): alpha-Synuclein 111-132
  PEPTIDE (27)..(48): alpha-Synuclein (G111-G132)
KKKISIXEIXXVIVXXIEXILFKKKKGILEDMPVDPDNEAYEMPSEEG

SEQ ID NO: 162 - protein, 44 aa, Artificial Sequence
  Synthetic peptide
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
  SITE (19)..(19): epsilon K as a spacer
  PEPTIDE (19)..(22): epsilon-K-KKK spacer
  PEPTIDE (23)..(44): alpha-Synuclein 111-132
  PEPTIDE (23)..(44): alpha-Synuclein (G111-G132)
XXXXXXXTXXXTXPXSXXKKKKGILEDMPVDPDNEAYEMPSEEG

SEQ ID NO: 163 - protein, 44 aa, Artificial Sequence
  Synthetic peptide
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
  SITE (19)..(19): epsilon K as a spacer
  PEPTIDE (19)..(22): epsilon-K-KKK spacer
  PEPTIDE (23)..(44): alpha-Synuclein (G111-G132)
KKKXXXXTRIXTIXXXXDKKKKGILEDMPVDPDNEAYEMPSEEG

SEQ ID NO: 164 - protein, 37 aa, Artificial Sequence
  Alpha-Synuclein peptide immunogen construct
  PEPTIDE (1)..(11): Influenza MP1_1 Th
  PEPTIDE (12)..(15): epsilon-K-KKK spacer
  SITE (12)..(12): epsilon-K
  PEPTIDE (16)..(37): alpha-Synuclein (G111-G132)
FVFTLTVPSERKKKKGILEDMPVDPDNEAYEMPSEEG

SEQ ID NO: 165 - protein, 41 aa, Artificial Sequence
  Alpha-Synuclein peptide immunogen construct
  PEPTIDE (1)..(15): Influenza MP1_2 Th
  PEPTIDE (16)..(19): epsilon-K-KKK spacer
  SITE (16)..(16): epsilon-K
  PEPTIDE (20)..(41): alpha-Synuclein (G111-G132)
SGPLKAEIAQRLEDVKKKKGILEDMPVDPDNEAYEMPSEEG

SEQ ID NO: 166 - protein, 35 aa, Artificial Sequence
  Alpha-Synuclein peptide immunogen construct
  PEPTIDE (1)..(9): Influenza NSP1 Th
  PEPTIDE (10)..(13): epsilon-K-KKK spacer
  SITE (10)..(10): epsilon-K
  PEPTIDE (14)..(35): alpha-Synuclein (G111-G132)
DRLRRDQKSKKKKGILEDMPVDPDNEAYEMPSEEG

SEQ ID NO: 167 - protein, 45 aa, Artificial Sequence
  Alpha-Synuclein peptide immunogen construct
  PEPTIDE (1)..(19): EBV BHRF1 Th
  PEPTIDE (20)..(23): epsilon-K-KKK spacer
  SITE (20)..(20): epsilon-K
  PEPTIDE (24)..(45): alpha-Synuclein (G111-G132)
AGLTLSLLVICSYLFISRGKKKKGILEDMPVDPDNEAYEMPSEEG

SEQ ID NO: 168 - protein, 41 aa, Artificial Sequence
  Alpha-Synuclein peptide immunogen construct
  PEPTIDE (1)..(15): Clostridium tetani TT1 Th
  PEPTIDE (16)..(19): epsilon-K-KKK spacer
  SITE (16)..(16): epsilon-K
  PEPTIDE (20)..(41): alpha-Synuclein (G111-G132)
QYIKANSKFIGITELKKKKGILEDMPVDPDNEAYEMPSEEG

SEQ ID NO: 169 - protein, 46 aa, Artificial Sequence
  Alpha-Synuclein peptide immunogen construct
  PEPTIDE (1)..(20): EBV EBNA-1 Th
  PEPTIDE (21)..(24): epsilon-K-KKK spacer
  SITE (21)..(21): epsilon-K
  PEPTIDE (25)..(46): alpha-Synuclein (G111-G132)
PGPLRESIVCYFMVFLQTHIKKKKGILEDMPVDPDNEAYEMPSEEG

SEQ ID NO: 170 - protein, 47 aa, Artificial Sequence
  Alpha-Synuclein peptide immunogen construct
  PEPTIDE (1)..(21): Clostridium tetani TT2 Th
  PEPTIDE (22)..(25): epsilon-K-KKK spacer
  SITE (22)..(22): epsilon-K
  PEPTIDE (26)..(47): alpha-Synuclein (G111-G132)
FNNFTVSFWLRVPKVSASHLEKKKKGILEDMPVDPDNEAYEMPSEEG

SEQ ID NO: 171 - protein, 42 aa, Artificial Sequence
  Alpha-Synuclein peptide immunogen construct
  PEPTIDE (1)..(16): Clostridium tetani TT3 Th
  PEPTIDE (17)..(20): epsilon-K-KKK spacer
  SITE (17)..(17): epsilon-K
  PEPTIDE (21)..(42): alpha-Synuclein (G111-G132)
KFIIKRYTPNNEIDSFKKKKGILEDMPVDPDNEAYEMPSEEG

SEQ ID NO: 172 - protein, 42 aa, Artificial Sequence
  Alpha-Synuclein peptide immunogen construct
  PEPTIDE (1)..(16): Clostridium tetani TT4 Th
  PEPTIDE (17)..(20): epsilon-K-KKK spacer
  SITE (17)..(17): epsilon-K
  PEPTIDE (21)..(42): alpha-Synuclein (G111-G132)
VSIDKFRIFCKALNPKKKKKGILEDMPVDPDNEAYEMPSEEG

SEQ ID NO: 173 - protein, 44 aa, Artificial Sequence
  Alpha-Synuclein peptide immunogen construct
  PEPTIDE (1)..(18): EBV CP Th
  PEPTIDE (19)..(22): epsilon-K-KKK spacer
  SITE (19)..(19): epsilon-K
  PEPTIDE (23)..(44): alpha-Synuclein (G111-G132)
VPGLYSPCRAFFNKEELLKKKKGILEDMPVDPDNEAYEMPSEEG

SEQ ID NO: 174 - protein, 40 aa, Artificial Sequence
  Alpha-Synuclein peptide immunogen construct
  PEPTIDE (1)..(14): HCMV IE1 Th
  PEPTIDE (15)..(18): epsilon-K-KKK spacer
  SITE (15)..(15): epsilon-K
  PEPTIDE (19)..(40): alpha-Synuclein (G111-G132)
DKREMWMACIKELHKKKKGILEDMPVDPDNEAYEMPSEEG

SEQ ID NO: 175 - protein, 41 aa, Artificial Sequence
  Alpha-Synuclein peptide immunogen construct
  PEPTIDE (1)..(15): EBV GP340 Th
  PEPTIDE (16)..(19): epsilon-K-KKK spacer
  SITE (16)..(16): epsilon-K
  PEPTIDE (20)..(41): alpha-Synuclein (G111-G132)
TGHGARTSTEPTTDYKKKKGILEDMPVDPDNEAYEMPSEEG

SEQ ID NO: 176 - protein, 39 aa, Artificial Sequence
  Alpha-Synuclein peptide immunogen construct
  PEPTIDE (1)..(13): EBV BPLF1 Th
  PEPTIDE (14)..(17): epsilon-K-KKK spacer
  SITE (14)..(14): epsilon-K
  PEPTIDE (18)..(39): alpha-Synuclein (G111-G132)
KELKRQYEKKLRQKKKKGILEDMPVDPDNEAYEMPSEEG

SEQ ID NO: 177 - protein, 37 aa, Artificial Sequence
  Alpha-Synuclein peptide immunogen construct
  PEPTIDE (1)..(11): EBV EBNA-2 Th
  PEPTIDE (12)..(15): epsilon-K-KKK spacer
  SITE (12)..(12): epsilon-K
  PEPTIDE (16)..(37): alpha-Synuclein (G111-G132)
TVFYNIPPMPLKKKKGILEDMPVDPDNEAYEMPSEEG

SEQ ID NO: 178 - protein, 59 aa, Artificial Sequence
  IgE-EMPD peptide immunogen construct
  PEPTIDE (1)..(39): IgE-EMPD (G1-C39)
  SITE (40)..(40): epsilon-K
  PEPTIDE (41)..(59): MvF5 Th (UBITh1)
GLAGGSAQSQRAPDRVLCHSGQQQGLPRAAGGSVPHPRCKISITEIKGVIVHRIETILF

SEQ ID NO: 179 - protein, 58 aa, Artificial Sequence
  IgE-EMPD peptide immunogen construct
  PEPTIDE (1)..(18): HBsAg3 Th (UBITh2)
  SITE (19)..(19): epsilon-K
  PEPTIDE (20)..(58): IgE-EMPD (G1-C39)
KKKIITITRIITIITTIDKGLAGGSAQSQRAPDRVLCHSGQQQGLPRAAGGSVPHPRC

SEQ ID NO: 180 - protein, 59 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (21)..(59): IgE-EMPD (G1-C39)
ISIXEIXXVIVXXIETILFKGLAGGSAQSQRAPDRVLCHSGQQQGLPRAAGGSVPHPRC

SEQ ID NO: 181 - protein, 57 aa, Artificial Sequence
  IgE-EMPD peptide immunogen construct
  PEPTIDE (1)..(17): Clostridium tetani1 Th
  SITE (18)..(18): epsilon-K
  PEPTIDE (19)..(57): IgE-EMPD (G1-C39)
KKQYIKANSKFIGITELKGLAGGSAQSQRAPDRVLCHSGQQQGLPRAAGGSVPHPRC

SEQ ID NO: 182 - protein, 55 aa, Artificial Sequence
  IgE-EMPD peptide immunogen construct
  PEPTIDE (1)..(15): MvF1 Th
  SITE (16)..(16): epsilon-K
  PEPTIDE (17)..(55): IgE-EMPD (G1-C39)
LSEIKGVIVHRLEGVKGLAGGSAQSQRAPDRVLCHSGQQQGLPRAAGGSVPHPRC

SEQ ID NO: 183 - protein, 64 aa, Artificial Sequence
  IgE-EMPD peptide immunogen construct
  PEPTIDE (1)..(24): Bordetella pertussis Th
  SITE (25)..(25): epsilon-K
  PEPTIDE (26)..(64): IgE-EMPD (G1-C39)
GAYARCPNGTRALTVAELRGNAELKGLAGGSAQSQRAPDRVLCHSGQQQGLPRAAGGSVPHPRC

SEQ ID NO: 184 - protein, 57 aa, Artificial Sequence
  IgE-EMPD peptide immunogen construct
  PEPTIDE (1)..(17): Clostridium tetani2 Th
  SITE (18)..(18): epsilon-K
  PEPTIDE (19)..(57): IgE-EMPD (G1-C39)
WVRDIIDDFTNESSQKTKGLAGGSAQSQRAPDRVLCHSGQQQGLPRAAGGSVPHPRC

SEQ ID NO: 185 - protein, 63 aa, Artificial Sequence
  IgE-EMPD peptide immunogen construct
  PEPTIDE (1)..(23): Diphtheria Th
  SITE (24)..(24): epsilon-K
  PEPTIDE (25)..(63): IgE-EMPD (G1-C39)
DSETADNLEKTVAALSILPGHGCKGLAGGSAQSQRAPDRVLCHSGQQQGLPRAAGGSVPHPRC

SEQ ID NO: 186 - protein, 61 aa, Artificial Sequence
  IgE-EMPD peptide immunogen construct
  PEPTIDE (1)..(21)
  SITE (22)..(22): epsilon-K
  PEPTIDE (23)..(61): IgE-EMPD (G1-C39)
DHEKKHAKMEKASSVFNVVNSKGLAGGSAQSQRAPDRVLCHSGQQQGLPRAAGGSVPHPRC

SEQ ID NO: 187 - protein, 57 aa, Artificial Sequence
  IgE-EMPD peptide immunogen construct
  PEPTIDE (1)..(17): Schistosoma mansoni Th
  SITE (18)..(18): epsilon-K
  PEPTIDE (19)..(57): IgE-EMPD (G1-C39)
KWFKTNAPNGVDEKHRHKGLAGGSAQSQRAPDRVLCHSGQQQGLPRAAGGSVPHPRC

SEQ ID NO: 188 - protein, 65 aa, Artificial Sequence
  IgE-EMPD peptide immunogen construct
  PEPTIDE (1)..(25): Cholera Toxin Th
  SITE (26)..(26): epsilon-K
  PEPTIDE (27)..(65): IgE-EMPD (G1-C39)
ALNIWDRFDVFCTLGATTGYLKGNSKGLAGGSAQSQRAPDRVLCHSGQQQGLPRAAGGSVPHPRC

SEQ ID NO: 189 - protein, 55 aa, Artificial Sequence
  IgE-EMPD peptide immunogen construct
  PEPTIDE (1)..(15): MvF2 Th
  SITE (16)..(16): epsilon-K
  PEPTIDE (17)..(55): IgE-EMPD (G1-C39)
ISEIKGVIVHKIEGIKGLAGGSAQSQRAPDRVLCHSGQQQGLPRAAGGSVPHPRC

SEQ ID NO: 190 - protein, 62 aa, Artificial Sequence
  Synthetic peptide
  PEPTIDE (1)..(22): KKKMvF3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
  SITE (23)..(23): epsilon K as a spacer
  PEPTIDE (24)..(62): IgE-EMPD (G1-C39)
KKKISIXEIXXVIVXXIEXILFKGLAGGSAQSQRAPDRVLCHSGQQQGLPRAAGGSVPHPRC

SEQ ID NO: 191 - protein, 58 aa, Artificial Sequence
  Synthetic peptide
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
  SITE (19)..(19): epsilon K as a spacer
  PEPTIDE (20)..(58): IgE-EMPD (G1-C39)
XXXXXXXTXXXTXPXSXXKGLAGGSAQSQRAPDRVLCHSGQQQGLPRAAGGSVPHPRC

SEQ ID NO: 192 - protein, 58 aa, Artificial Sequence
  Synthetic peptide
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
  SITE (19)..(19): epsilon K as a spacer
  PEPTIDE (20)..(58): IgE-EMPD (G1-C39)
KKKXXXXTRIXTIXXXXDKGLAGGSAQSQRAPDRVLCHSGQQQGLPRAAGGSVPHPRC

SEQ ID NO: 193 - protein, 51 aa, Artificial Sequence
  IgE-EMPD peptide immunogen construct
  PEPTIDE (1)..(11): Influenza MP1_1 Th
  SITE (12)..(12): epsilon-K
  PEPTIDE (13)..(51): IgE-EMPD (G1-C39)
FVFTLTVPSERKGLAGGSAQSQRAPDRVLCHSGQQQGLPRAAGGSVPHPRC

SEQ ID NO: 194 - protein, 55 aa, Artificial Sequence
  IgE-EMPD peptide immunogen construct
  PEPTIDE (1)..(15): Influenza MP1_2 Th
  SITE (16)..(16): epsilon-K
  PEPTIDE (17)..(55): IgE-EMPD (G1-C39)
SGPLKAEIAQRLEDVKGLAGGSAQSQRAPDRVLCHSGQQQGLPRAAGGSVPHPRC

SEQ ID NO: 195 - protein, 49 aa, Artificial Sequence
  IgE-EMPD peptide immunogen construct
  PEPTIDE (1)..(9): Influenza NSP1 Th
  SITE (10)..(10): epsilon-K
  PEPTIDE (11)..(49): IgE-EMPD (G1-C39)
DRLRRDQKSKGLAGGSAQSQRAPDRVLCHSGQQQGLPRAAGGSVPHPRC

SEQ ID NO: 196 - protein, 59 aa, Artificial Sequence
  IgE-EMPD peptide immunogen construct
  PEPTIDE (1)..(19): EBV BHRF1 Th
  SITE (20)..(20): epsilon-K
  PEPTIDE (21)..(59): IgE-EMPD (G1-C39)
AGLTLSLLVICSYLFISRGKGLAGGSAQSQRAPDRVLCHSGQQQGLPRAAGGSVPHPRC

SEQ ID NO: 197 - protein, 55 aa, Artificial Sequence
  IgE-EMPD peptide immunogen construct
  PEPTIDE (1)..(15): Clostridium tetani TT1 Th
  SITE (16)..(16): epsilon-K
  PEPTIDE (17)..(55): IgE-EMPD (G1-C39)
QYIKANSKFIGITELKGLAGGSAQSQRAPDRVLCHSGQQQGLPRAAGGSVPHPRC

SEQ ID NO: 198 - protein, 60 aa, Artificial Sequence
  IgE-EMPD peptide immunogen construct
  PEPTIDE (1)..(20): EBV EBNA-1 Th
  SITE (21)..(21): epsilon-K
  PEPTIDE (22)..(60): IgE-EMPD (G1-C39)
PGPLRESIVCYFMVFLQTHIKGLAGGSAQSQRAPDRVLCHSGQQQGLPRAAGGSVPHPRC

SEQ ID NO: 199 - protein, 61 aa, Artificial Sequence
  IgE-EMPD peptide immunogen construct
  PEPTIDE (1)..(21): Clostridium tetani TT2 Th
  SITE (22)..(22): epsilon-K
  PEPTIDE (23)..(61): IgE-EMPD (G1-C39)
FNNFTVSFWLRVPKVSASHLEKGLAGGSAQSQRAPDRVLCHSGQQQGLPRAAGGSVPHPRC

SEQ ID NO: 200 - protein, 56 aa, Artificial Sequence
  IgE-EMPD peptide immunogen construct
  PEPTIDE (1)..(16): Clostridium tetani TT3 Th
  SITE (17)..(17): epsilon-K
  PEPTIDE (18)..(56): IgE-EMPD (G1-C39)
KFIIKRYTPNNEIDSFKGLAGGSAQSQRAPDRVLCHSGQQQGLPRAAGGSVPHPRC

SEQ ID NO: 201 - protein, 56 aa, Artificial Sequence
  IgE-EMPD peptide immunogen construct
  PEPTIDE (1)..(16): Clostridium tetani TT4 Th
  SITE (17)..(17): epsilon-K
  PEPTIDE (18)..(56): IgE-EMPD (G1-C39)
VSIDKFRIFCKALNPKKGLAGGSAQSQRAPDRVLCHSGQQQGLPRAAGGSVPHPRC

SEQ ID NO: 202 - protein, 58 aa, Artificial Sequence
  IgE-EMPD peptide immunogen construct
  PEPTIDE (1)..(18): EBV CP Th
  SITE (19)..(19): epsilon-K
  PEPTIDE (20)..(58): IgE-EMPD (G1-C39)
VPGLYSPCRAFFNKEELLKGLAGGSAQSQRAPDRVLCHSGQQQGLPRAAGGSVPHPRC

SEQ ID NO: 203 - protein, 54 aa, Artificial Sequence
  IgE-EMPD peptide immunogen construct
  PEPTIDE (1)..(14): HCMV IE1 Th
  SITE (15)..(15): epsilon-K
  PEPTIDE (16)..(54): IgE-EMPD (G1-C39)
DKREMWMACIKELHKGLAGGSAQSQRAPDRVLCHSGQQQGLPRAAGGSVPHPRC

SEQ ID NO: 204 - protein, 55 aa, Artificial Sequence
  IgE-EMPD peptide immunogen construct
  PEPTIDE (1)..(15): EBV GP340 Th
  SITE (16)..(16): epsilon-K
  PEPTIDE (17)..(55): IgE-EMPD (G1-C39)
TGHGARTSTEPTTDYKGLAGGSAQSQRAPDRVLCHSGQQQGLPRAAGGSVPHPRC

SEQ ID NO: 205 - protein, 53 aa, Artificial Sequence
  IgE-EMPD peptide immunogen construct
  PEPTIDE (1)..(13): EBV BPLF1 Th
  SITE (14)..(14): epsilon-K
  PEPTIDE (15)..(53): IgE-EMPD (G1-C39)
KELKRQYEKKLRQKGLAGGSAQSQRAPDRVLCHSGQQQGLPRAAGGSVPHPRC

SEQ ID NO: 206 - protein, 51 aa, Artificial Sequence
  IgE-EMPD peptide immunogen construct
  PEPTIDE (1)..(11): EBV EBNA-2 Th
  SITE (12)..(12): epsilon-K
  PEPTIDE (13)..(51): IgE-EMPD (G1-C39)
TVFYNIPPMPLKGLAGGSAQSQRAPDRVLCHSGQQQGLPRAAGGSVPHPRC

SEQ ID NO: 207 - protein, 34 aa, Artificial Sequence
  IL-6 peptide immunogen construct
  PEPTIDE (1)..(19): MvF5 Th (UBITh1)
  SITE (20)..(20): epsilon-K
  PEPTIDE (20)..(23): epsilon-K-KKK spacer
  PEPTIDE (24)..(34): IL6 (C73-C83)
ISITEIKGVIVHRIETILFKKKKCFQSGFNEETC

SEQ ID NO: 208 - protein, 33 aa, Artificial Sequence
  IL-6 peptide immunogen construct
  PEPTIDE (1)..(18): HBsAg3 Th (UBITh2)
  SITE (19)..(19): epsilon-K
  PEPTIDE (19)..(22): epsilon-K-KKK spacer
  PEPTIDE (23)..(33): IL6 (C73-C83)
KKKIITITRIITIITTIDKKKKCFQSGFNEETC

SEQ ID NO: 209 - protein, 34 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  PEPTIDE (20)..(23): epsilon-K-KKK spacer
  SITE (20)..(20): epsilon-K
  PEPTIDE (24)..(34): IL6 (C73-C83)
ISIXEIXXVIVXXIETILFKKKKCFQSGFNEETC

SEQ ID NO: 210 - protein, 32 aa, Artificial Sequence
  IL-6 peptide immunogen construct
  PEPTIDE (1)..(17): Clostridium tetani1 Th
  PEPTIDE (18)..(21): KKK-epsilon-K spacer
  SITE (21)..(21): epsilon-K
  PEPTIDE (22)..(32): IL6 (C73-C83)
KKQYIKANSKFIGITELKKKKCFQSGFNEETC

SEQ ID NO: 211 - protein, 30 aa, Artificial Sequence
  IL-6 peptide immunogen construct
  PEPTIDE (1)..(15): MvF1 Th
  PEPTIDE (16)..(19): KKK-epsilon-K spacer
  SITE (19)..(19): epsilon-K
  PEPTIDE (20)..(30): IL6 (C73-C83)
LSEIKGVIVHRLEGVKKKKCFQSGFNEETC

SEQ ID NO: 212 - protein, 39 aa, Artificial Sequence
  IL-6 peptide immunogen construct
  PEPTIDE (1)..(24): Bordetella pertussis Th
  PEPTIDE (25)..(28): KKK-epsilon-K spacer
  SITE (28)..(28): epsilon-K
  PEPTIDE (29)..(39): IL6 (C73-C83)
GAYARCPNGTRALTVAELRGNAELKKKKCFQSGFNEETC

SEQ ID NO: 213 - protein, 32 aa, Artificial Sequence
  IL-6 peptide immunogen construct
  PEPTIDE (1)..(17): Clostridium tetani2 Th
  PEPTIDE (18)..(21): KKK-epsilon-K spacer
  SITE (21)..(21): epsilon-K
  PEPTIDE (22)..(32): IL6 (C73-C83)
WVRDIIDDFTNESSQKTKKKKCFQSGFNEETC

SEQ ID NO: 214 - protein, 38 aa, Artificial Sequence
  IL-6 peptide immunogen construct
  PEPTIDE (1)..(23): Diphtheria Th
  PEPTIDE (24)..(27): KKK-epsilon-K spacer
  SITE (27)..(27): epsilon-K
  PEPTIDE (28)..(38): IL6 (C73-C83)
DSETADNLEKTVAALSILPGHGCKKKKCFQSGFNEETC

SEQ ID NO: 215 - protein, 36 aa, Artificial Sequence
  IL-6 peptide immunogen construct
  PEPTIDE (1)..(21): Plasmodium falciparum Th
  PEPTIDE (22)..(25): KKK-epsilon-K spacer
  SITE (25)..(25): epsilon-K
  PEPTIDE (26)..(36): IL6 (C73-C83)
DHEKKHAKMEKASSVFNVVNSKKKKCFQSGFNEETC

SEQ ID NO: 216 - protein, 32 aa, Artificial Sequence
  IL-6 peptide immunogen construct
  PEPTIDE (1)..(17): Schistosoma mansoni Th
  PEPTIDE (18)..(21): KKK-epsilon-K spacer
  SITE (21)..(21): epsilon-K
  PEPTIDE (22)..(32): IL6 (C73-C83)
KWFKTNAPNGVDEKHRHKKKKCFQSGFNEETC

SEQ ID NO: 217 - protein, 40 aa, Artificial Sequence
  IL-6 peptide immunogen construct
  PEPTIDE (1)..(25): Cholera Toxin Th
  PEPTIDE (26)..(29): KKK-epsilon-K spacer
  SITE (29)..(29): epsilon-K
  PEPTIDE (30)..(40): IL6 (C73-C83)
ALNIWDRFDVFCTLGATTGYLKGNSKKKKCFQSGFNEETC

SEQ ID NO: 218 - protein, 30 aa, Artificial Sequence
  IL-6 peptide immunogen construct
  PEPTIDE (1)..(15): MvF2 Th
  PEPTIDE (16)..(19): KKK-epsilon-K spacer
  SITE (19)..(19): epsilon-K
  PEPTIDE (20)..(30): IL6 (C73-C83)
ISEIKGVIVHKIEGIKKKKCFQSGFNEETC

SEQ ID NO: 219 - protein, 37 aa, Artificial Sequence
  Synthetic peptide
  PEPTIDE (1)..(22): KKKMvF3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
  PEPTIDE (23)..(26): KKK-epsilon-K spacer
  SITE (26)..(26): epsilon-K
  PEPTIDE (27)..(37): IL6 (C73-C83)
KKKISIXEIXXVIVXXIEXILFKKKKCFQSGFNEETC

SEQ ID NO: 220 - protein, 33 aa, Artificial Sequence
  Synthetic peptide
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
  PEPTIDE (19)..(22): KKK-epsilon-K spacer
  SITE (22)..(22): epsilon-K
  PEPTIDE (23)..(33): IL6 (C73-C83)
XXXXXXXTXXXTXPXSXXKKKKCFQSGFNEETC

SEQ ID NO: 221 - protein, 33 aa, Artificial Sequence
  Synthetic peptide
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
  PEPTIDE (19)..(22): KKK-epsilon-K spacer
  SITE (22)..(22): epsilon-K
  PEPTIDE (23)..(33): IL6 (C73-C83)
KKKXXXXTRIXTIXXXXDKKKKCFQSGFNEETC

SEQ ID NO: 222 - protein, 26 aa, Artificial Sequence
  IL-6 peptide immunogen construct
  PEPTIDE (1)..(11): Influenza MP1_1 Th
  PEPTIDE (12)..(15): KKK-epsilon-K spacer
  SITE (15)..(15): epsilon-K
  PEPTIDE (16)..(26): IL6 (C73-C83)
FVFTLTVPSERKKKKCFQSGFNEETC

SEQ ID NO: 223 - protein, 30 aa, Artificial Sequence
  IL-6 peptide immunogen construct
  PEPTIDE (1)..(15): Influenza MP1_2 Th
  PEPTIDE (16)..(19): KKK-epsilon-K spacer
  SITE (19)..(19): epsilon-K
  PEPTIDE (20)..(30): IL6 (C73-C83)
SGPLKAEIAQRLEDVKKKKCFQSGFNEETC

SEQ ID NO: 224 - protein, 24 aa, Artificial Sequence
  IL-6 peptide immunogen construct
  PEPTIDE (1)..(9): Influenza NSP1 Th
  PEPTIDE (10)..(13): KKK-epsilon-K spacer
  SITE (13)..(13): epsilon-K
  PEPTIDE (14)..(24): IL6 (C73-C83)
DRLRRDQKSKKKKCFQSGFNEETC

SEQ ID NO: 225 - protein, 34 aa, Artificial Sequence
  IL-6 peptide immunogen construct
  PEPTIDE (1)..(19): EBV BHRF1 Th
  PEPTIDE (20)..(23): KKK-epsilon-K spacer
  SITE (23)..(23): epsilon-K
  PEPTIDE (24)..(34): IL6 (C73-C83)
AGLTLSLLVICSYLFISRGKKKKCFQSGFNEETC

SEQ ID NO: 226 - protein, 30 aa, Artificial Sequence
  IL-6 peptide immunogen construct
  PEPTIDE (1)..(15): Clostridium tetani TT1 Th
  PEPTIDE (16)..(19): KKK-epsilon-K spacer
  SITE (19)..(19): epsilon-K
  PEPTIDE (20)..(30): IL6 (C73-C83)
QYIKANSKFIGITELKKKKCFQSGFNEETC

SEQ ID NO: 227 - protein, 35 aa, Artificial Sequence
  IL-6 peptide immunogen construct
  PEPTIDE (1)..(20): EBV EBNA-1 Th
  PEPTIDE (21)..(24): KKK-epsilon-K spacer
  SITE (24)..(24): epsilon-K
  PEPTIDE (25)..(35): IL6 (C73-C83)
PGPLRESIVCYFMVFLQTHIKKKKCFQSGFNEETC

SEQ ID NO: 228 - protein, 36 aa, Artificial Sequence
  IL-6 peptide immunogen construct
  PEPTIDE (1)..(21): Clostridium tetani TT2 Th
  PEPTIDE (22)..(25): KKK-epsilon-K spacer
  SITE (25)..(25): epsilon-K
  PEPTIDE (26)..(36): IL6 (C73-C83)
FNNFTVSFWLRVPKVSASHLEKKKKCFQSGFNEETC

SEQ ID NO: 229 - protein, 31 aa, Artificial Sequence
  IL-6 peptide immunogen construct
  PEPTIDE (1)..(16): Clostridium tetani TT3 Th
  PEPTIDE (17)..(20): KKK-epsilon-K spacer
  SITE (20)..(20): epsilon-K
  PEPTIDE (21)..(31): IL6 (C73-C83)
KFIIKRYTPNNEIDSFKKKKCFQSGFNEETC

SEQ ID NO: 230 - protein, 31 aa, Artificial Sequence
  IL-6 peptide immunogen construct
  PEPTIDE (1)..(16): Clostridium tetani TT4 Th
  PEPTIDE (17)..(20): KKK-epsilon-K spacer
  SITE (20)..(20): epsilon-K
  PEPTIDE (21)..(31): IL6 (C73-C83)
VSIDKFRIFCKALNPKKKKKCFQSGFNEETC

SEQ ID NO: 231 - protein, 33 aa, Artificial Sequence
  IL-6 peptide immunogen construct
  PEPTIDE (1)..(18): EBV CP Th
  PEPTIDE (19)..(22): KKK-epsilon-K spacer
  SITE (22)..(22): epsilon-K
  PEPTIDE (23)..(33): IL6 (C73-C83)
VPGLYSPCRAFFNKEELLKKKKCFQSGFNEETC

SEQ ID NO: 232 - protein, 29 aa, Artificial Sequence
  IL-6 peptide immunogen construct
  PEPTIDE (1)..(14): HCMV IE1 Th
  PEPTIDE (15)..(18): KKK-epsilon-K spacer
  SITE (18)..(18): epsilon-K
  PEPTIDE (19)..(29): IL6 (C73-C83)
DKREMWMACIKELHKKKKCFQSGFNEETC

SEQ ID NO: 233 - protein, 30 aa, Artificial Sequence
  IL-6 peptide immunogen construct
  PEPTIDE (1)..(15): EBV GP340 Th
  PEPTIDE (16)..(19): KKK-epsilon-K spacer
  SITE (19)..(19): epsilon-K
  PEPTIDE (20)..(30): IL6 (C73-C83)
TGHGARTSTEPTTDYKKKKCFQSGFNEETC

SEQ ID NO: 234 - protein, 28 aa, Artificial Sequence
  IL-6 peptide immunogen construct
  PEPTIDE (1)..(13): EBV BPLF1 Th
  PEPTIDE (14)..(17): KKK-epsilon-K spacer
  SITE (17)..(17): epsilon-K
  PEPTIDE (18)..(28): IL6 (C73-C83)
KELKRQYEKKLRQKKKKCFQSGFNEETC

SEQ ID NO: 235 - protein, 26 aa, Artificial Sequence
  IL-6 peptide immunogen construct
  PEPTIDE (1)..(11): EBV EBNA-2 Th
  PEPTIDE (12)..(15): KKK-epsilon-K spacer
  SITE (15)..(15): epsilon-K
  PEPTIDE (16)..(26): IL6 (C73-C83)
TVFYNIPPMPLKKKKCFQSGFNEETC

SEQ ID NO: 236 - protein, 43 aa, Artificial Sequence
  DPR peptide immunogen construct
  PEPTIDE (1)..(20): (GA) 10 repeats
  PEPTIDE (21)..(24): KKK-epsilon-K spacer
  SITE (24)..(24): epsilon-K
  PEPTIDE (25)..(43): UBITh1
GAGAGAGAGAGAGAGAGAGAKKKKISITEIKGVIVHRIETILF

SEQ ID NO: 237 - protein, 53 aa, Artificial Sequence
  DPR peptide immunogen construct
  PEPTIDE (1)..(30): (GA) 15 repeats
  PEPTIDE (31)..(34): KKK-epsilon-K spacer
  SITE (34)..(34): epsilon-K
  PEPTIDE (35)..(53): UBITh1
GAGAGAGAGAGAGAGAGAGAGAGAGAGAGAKKKKISITEIKGVIVHRIETILF

SEQ ID NO: 238 - protein, 73 aa, Artificial Sequence
  DPR peptide immunogen construct
  PEPTIDE (1)..(50): (GA) 25 repeats
  PEPTIDE (51)..(54): KKK-epsilon-K spacer
  SITE (54)..(54): epsilon-K
  PEPTIDE (55)..(73): UBITh1
GAGAGAGAGAGAGAGAGAGAGAGAGAGAGAGAGAGAGAGAGAGAGAGAGAKKKKISITEIKGVIVHRIETILF

SEQ ID NO: 239 - protein, 27 aa, Artificial Sequence
  LHRH peptide immunogen construct
  PEPTIDE (1)..(17): TT1 Th
  PEPTIDE (18)..(27): LHRH
KKQYIKANSKFIGITELEHWSYGLRPG

SEQ ID NO: 240 - protein, 45 aa, Artificial Sequence
  LHRH peptide immunogen construct
  PEPTIDE (1)..(16): Invasin
  SITE (17)..(18): GG spacer
  PEPTIDE (19)..(33): MVF Th
  SITE (34)..(35): GG spacer
  PEPTIDE (36)..(45): LHRH
TAKSKKFPSYTATYQFGGLSEIKGVIVHRLEGVGGEHWSYGLRPG

SEQ ID NO: 241 - protein, 45 aa, Artificial Sequence
  LHRH peptide immunogen construct
  PEPTIDE (1)..(16): Invasin
  SITE (17)..(18): GG spacer
  PEPTIDE (19)..(33): HBsAg Th
  SITE (34)..(35): GG spacer
  PEPTIDE (36)..(45): LHRH
TAKSKKFPSYTATYQFGGFFLLTRILTIPQSLEGGEHWSYGLRPG

SEQ ID NO: 242 - protein, 11 aa, Artificial Sequence
  IL-6 (72-82) (rat counterpart)
  PEPTIDE (1)..(11): IL-6 (72-82) rat counterpart
CFQTGYNQEIC

SEQ ID NO: 243 - protein, 34 aa, Artificial Sequence
  Synthetic peptide
  PEPTIDE (1)..(19): MvF 4 Th (UBITh3)
  SITE (4)..(4): S or T
  SITE (7)..(7): K or R
  SITE (8)..(8): G or T
  SITE (12)..(12): H or T
  SITE (13)..(13): K or R
  PEPTIDE (20)..(23): epsilon K-KKK spacer
  SITE (20)..(20): epsilon-K
  PEPTIDE (24)..(34): rat counterpart IL-6 (72-82)
ISIXEIXXVIVXXIETILFKKKKCFQTGYNQEIC

(19) United States
(12) Patent Application Publication
(10) Pub. No.: US 2023/0218748 A1
(43) Pub. Date: Jul. 13, 2023

(54) ARTIFICIAL PROMISCUOUS T HELPER CELL EPITOPES AS IMMUNE STIMULATORS FOR SYNTHETIC PEPTIDE IMMUNOGENS

(71) Applicant: UNITED BIOMEDICAL, INC., Hauppauge, NY (US)

(72) Inventor: Chang Yi WANG, Cold Spring Harbor, NY (US)

(21) Appl. No.: 17/416,306

(22) PCT Filed: Dec. 19, 2019
(86) PCT No.: PCT/US2019/067532

§ 371 Date: Jun. 18, 2021

Related U.S. Application Data
(60) Provisional application No. 62/782,253, filed on Dec. 19, 2018.

(51) Int. Cl. A61K 39/245; C07K 14/47; A61K 39/21; A61K 39/135; A61K 39/12; A61K 39/187; A61P 31/22; A61P 31/18; A61P 31/14

(52) U.S. Cl. CPC A61K 39/245; C07K 14/47; A61K 39/21; A61K 39/135; A61K 39/12; A61K 39/187; A61P 31/22; A61P 31/18; A61P 31/14; A61K 2039/6031

(57) ABSTRACT

The present invention is directed to novel promiscuous and artificial T helper cell epitopes (Th epitopes) designed to provide optimum immunogenicity of a target antigenic site. The target antigenic site can include a B cell epitope, a CTL epitope, a peptide hapten, a non-peptide hapten, or any immunologically reactive analogue thereof. The disclosed Th epitopes, when covalently linked to a target antigenic site in a peptide immunogen construct, elicit a strong B cell antibody response or an effector T cell response to the target antigenic site. The Th epitopes are immunosilent on their own, i.e., little, if any, of the antibodies generated by the peptide immunogen constructs will be directed towards the Th epitope, thus allowing a very focused immune response directed to the targeted antigenic site. The promiscuous artificial Th epitopes provide effective and safe peptide immunogens that do not generate inflammatory, anti-self, cell-mediated immune responses following administration.

The present application is a PCT International Application that claims the benefit of U.S. Provisional Application Ser. No. 62/782,253, filed Dec. 19, 2018, which is incorporated herein by reference in its entirety.

### BACKGROUND

Immune responses require the cooperative interaction between antigen-presenting cells and T helper (Th) cells. The elicitation of an effective antibody response requires that antigen-presenting cells recognize the target antigenic site of a subject immunogen and that the T helper cells recognize a T helper cell epitope. Generally, the T helper epitope on a subject immunogen is different from its B cell epitope(s) or related effector T cell (e.g., cytotoxic T lymphocyte or CTL) epitope(s). The B cell and related effector T cell epitopes are sites on a desired target immunogen that is recognized by B cells and related cells, which results in the production of antibodies or cytokines against the desired target site. The natural conformation of the target determines the site to which the antibody or related effector T cell directly binds. Evocation of a Th cell response requires a Th cell receptor to recognize a complex on the membrane of an antigen-presenting cell that is formed between a processed peptide fragment of a target protein and an associated class II major histocompatibility complex (MHC). Thus, peptide processing of the target protein and three-way recognition are required for a Th cell response. The three part complex is difficult to define because 1) the critical MHC class II contact residues are variably positioned within different MHC binding peptides (Th epitopes); 2) the different MHC binding peptides have variable lengths and different amino acid sequences; and 3) MHC class II molecules can be highly diverse depending on the genetic make-up of the host. The immune responsiveness to a particular Th epitope is, in part, determined by the MHC genes of the host, and the reactivity of Th epitopes differ among individuals of a population. Th epitopes that are reactive across species and individuals (i.e., promiscuous Th epitopes) within a single species are difficult to identify.

Multiple factors are required for each component step of T cell recognition, such as appropriate peptide processing by the antigen-processing cell, presentation of the peptide by a genetically determined class II MHC molecule, and recognition of an MHC molecule and peptide complex by the receptor on Th cells. The requirements for promiscuous Th epitope recognition for providing broad responsiveness can be difficult to determine.

It is clear that for the induction of antibodies and related cytokines against immune responses, the immunogen must comprise both the B cell epitope/effector T cell epitope and Th cell determinant(s). Commonly, a carrier protein (e.g., keyhole limpet hemocyanin; KLH) is coupled to a target immunogen to provide the Th response in order to increase the immunogenicity of the target immunogen. However, there are many disadvantages with using large carrier proteins to enhance the immunogenicity of a target immunogen. In particular, it is difficult to manufacture a well-defined, safe, and effective peptide-carrier protein conjugates because (a) chemical coupling involves reactions that can result in heterogeneity in size and composition, e.g., conjugation with glutaraldehyde (Borras-Cuesta et al., Eur J Immunol, 1987; 17: 1213-1215); (b) the carrier protein introduces a potential for undesirable immune responses such as allergic and autoimmune reactions (Bixler et al., WO 89/06974); (c) the large peptide-carrier protein elicits irrelevant immune responses predominantly misdirected to the carrier protein rather than the target site (Cease et al., Proc Natl Acad Sci USA, 1987; 84: 4249-4253); and (d) the carrier protein also introduces a potential for epitopic suppression in a host that had previously been immunized with an immunogen comprising the same carrier protein. When a host is subsequently immunized with another immunogen wherein the same carrier protein is coupled to a different hapten, the resultant immune response is enhanced for the carrier protein but inhibited for the hapten (Schutze et al., J Immunol, 1985; 135: 2319-2322).

To avoid the risks described above, it is desirable to elicit T cell help without the use of traditional carrier proteins.

### SUMMARY OF THE INVENTION

The present disclosure provides promiscuous artificial T helper cell (Th) epitopes for stimulating functional site-directed antibodies against target antigen for preventative and therapeutic use. The present disclosure is also directed to peptide immunogen constructs that contain the Th epitopes, compositions containing the Th epitopes, methods of making and using the Th epitopes, and antibodies produced by peptide immunogen constructs containing the Th epitopes.

The disclosed artificial T helper cell (Th) epitopes can be linked to a B cell epitope(s) and/or effector T cell epitope(s) (“target antigenic site(s)”) through an optional spacer to produce a peptide immunogen construct. The disclosed Th epitopes impart to the peptide immunogen the ability to induce a strong T helper cell-mediated immune response with the production of high level of antibodies and/or cellular responses against the target antigenic site. The disclosed peptide immunogen constructs provide for the advantageous replacement of large carrier proteins and pathogen-derived T helper cell sites in peptide immunogens with the disclosed artificial Th epitopes designed specifically to improve the immunogenicity of the target antigenic site. The relatively short peptide immunogen constructs containing the disclosed Th epitopes elicit a high level of antibodies and/or effector cell related cytokines to a specific target antigenic site without causing a significant inflammatory response or immune response against the Th epitope.

The immune response elicited by the peptide immunogen constructs (including antibody titers, Cₘₐₓ, onset of antibody production, duration of response, etc.) can be modulated by varying: (a) the choice of the Th epitope that is chemically linked to the B cell epitope, (b) the length of the B cell epitope, (c) the adjuvant that is used in the formulation containing the peptide immunogen construct, and/or (d) the dosing regimen including dosage per immunization and the prime and boost time points for each immunization. Therefore, specific immune responses to target antigenic sites can be designed using the disclosed Th epitopes, which can facilitate the tailoring of personalized medical treatment to the individual characteristics of any patient or subject.

Peptide immunogen constructs containing the disclosed artificial Th epitopes of the present invention can be represented by the following formulae:

(A)ₙ-(Target antigenic site)-(B)ₒ-(Th)ₘ-(A)ₙ-X

or

(A)ₙ-(Th)ₘ-(B)ₒ-(Target antigenic site)-(A)ₙ-X

or

(A)ₙ-(Th)ₘ-(B)ₒ-(Target antigenic site)-(B)ₒ-(Th)ₘ-(A)ₙ-X

or

{(A)ₙ-(Th)ₚ-(B)ₒ-(Target antigenic site)-(B)ₒ-(Th)ₚ-(A)ₙ-X}m

wherein:
each A is independently an amino acid;
each B is independently a heterologous spacer;
each Th is independently an artificial Th epitope;
Target antigenic site is a B cell epitope, a CTL epitope, a peptide hapten, a non-peptide hapten, or an immunologically reactive analogue thereof;
X is an amino acid, α-COOH, or α-CONH₂;
n is 0, 1, 2, 3, 4, 5, 6, 7, 8, 9, or 10;
m is 1, 2, 3, or 4;
o is 0, 1, 2, 3, 4, 5, 6, 7, 8, 9, or 10; and
p is 0, 1, 2, 3, or 4.

An example of a peptide hapten as a target antigenic site is amino acids 1-14 of the beta-amyloid (Aβ) protein (Aβ₁₋₁₄) (SEQ ID NO: 56). Examples of a non-peptide hapten include a tumor associated carbohydrate antigen (TACA) or a small molecule drug.

The present disclosure is also directed to compositions that comprise peptide immunogen constructs containing the artificial Th epitopes. Such compositions are capable of evoking antibody responses in an immunized host to a desired target antigenic site. The target antigenic site may be derived from pathogenic organisms, self-antigens that are normally immunosilent, or tumor-associated targets.

The disclosed compositions are useful in many diverse medical and veterinary applications, including vaccines to provide protective immunity from infectious disease, immunotherapies for the treatment of disorders resulting from the malfunction of normal physiological processes, immunotherapies for the treatment of cancer, and agents to desirably intervene in and modify normal physiological processes.

Some of the targets antigens that may be covalently linked to the Th epitopes of the present invention include portions of: beta-amyloid (Aβ) for the treatment of Alzheimer's Disease, alpha-synuclein (α-Syn) for the treatment of Parkinson's Disease, the extracellular membrane-proximal domain of membrane-bound IgE (or IgE EMPD) for the treatment of allergic disease, Tau for the treatment of tauopathies including Alzheimer's Disease, and Interleukin-31 (IL-31) for the treatment of atopic dermatitis, to name a few. More specifically, the target antigens include Aβ₁₋₁₄ (as described in U.S. Pat. No. 9,102,752), α-Syn₁₁₁₋₁₃₂ (as described in International PCT Application No. PCT/US2018/037938), IgE EMPD₁₋₃₉ (as described in International PCT Application No. PCT/US2017/069174), Tau₃₇₉₋₄₀₈ (as described in International PCT Application No. PCT/US2018/057840), and IL-3₁₉₇₋₁₄₄ (as described in International PCT Application No. PCT/US2018/065025) and those target antigenic sites described in Table 3A and Table 3B.

The present disclosure also provides methods for preventing and/or treating a disease or condition in a subject by administering a peptide immunogen construct (comprising a disclosed artificial Th epitope and an antigen-presenting epitope) to a subject in need thereof. In some embodiments, the peptide immunogen constructs produce an immunogenic inflammatory response in the subject that is at least about 3-fold lower than an immunogenic inflammatory response of a positive control, as shown in Example 12.

The present disclosure is also directed to antibodies produced by peptide immunogen constructs containing the disclosed artificial Th epitopes. The antibodies produced by the peptide immunogen constructs are highly specific to the target antigenic site and not the artificial Th epitopes.

### REFERENCES

Each patent, publication, and non-patent literature cited in the application is hereby incorporated by reference in its entirety as if each was incorporated by reference individually.
1. BORRAS-CUESTA, F., et al., “Engineering of immunogenic peptides by co-linear synthesis of determinants recognized by B and T cells”, Eur. J. Immunol., 17:1213-1215 (1987).
2. CEASE, K. B., et al., “Helper T-cell antigenic site identification in the acquired immunodeficiency syndrome virus gp120 envelope protein and induction of immunity in mice to the native protein using a 16-residue synthetic peptide”, Proc. Natl. Acad. Sci. USA, 84:4249-4253 (1987).
3. CHIANG, H-L., et al., “A novel synthetic bipartite carrier protein for developing glycotope-based vaccines”, Vaccine, 30(52):7573-7581 (2012).
4. DANISHEFSKY, S. J., et al., “Development of Globo-H Cancer Vaccine”, Acc. Chem. Res. 48(3):643-652 (2015).
5. HUANG, C. Y, et al., “Carbohydrate microarray for profiling the antibodies interacting with Globo H tumor antigen”, Proc. Natl. Acad. Sci. USA 103(1):15-20 (2006).
6. JACQUES, S., et al., “Chemoenzymatic synthesis of GM3 and GM2 gangliosides containing a truncated ceramide functionalized for glycoconjugate synthesis and solid phase applications”, J. Am. Chem. Soc., 134(10):4521-4 (2012).
7. KATIAL, R. K., et al., “Cytokine Production in Cell Culture by Peripheral Blood Mononuclear Cells from Immunocompetent Hosts”, Clin. Diag. Lab. Immunol., 5(1):78-81 (1998).
8. KUDUK, S. D., et al., “Synthetic and Immunological Studies on Clustered Modes of Mucin-Related Tn and TF O-Linked Antigens: The Preparation of a Glycopeptide-Based Vaccine for Clinical Trials against Prostate Cancer”; J. Am. Chem. Soc., 120(48):12474-85 (1998).
9. KUZNIK, G., et al., “Chemical and enzymatic synthesis of high-affinity selectin ligands”, Bioorganic & Medicinal Chemistry Letters, 7(5):577-580 (1997).
10. MEISTER, G. E., et al., “Two novel T cell epitope prediction algorithms based on MHC-binding motifs; comparison of predicted and published epitopes from Mycobacterium tuberculosis and HIV protein sequences”, Vaccine, 13(6):581-591 (1995).
11. PARTIDOS, C. D., et al., “Immune responses in mice following immunization with chimeric synthetic peptides representing B and T cell epitopes of measles virus proteins”, J. of Gen. Virology, 72:1293-1299 (1991).
12. ROTHBARD, J. B., et al., “A sequence pattern common to T cell epitopes”, EMBO J., 7(1):93-101 (1988).
13. SCHUTZE, M. P., et al., “Carrier-induced epitopic suppression, a major issue for future synthetic vaccines”, J. Immunol., 135(4):2319-2322 (1985).
14. TOYOKUNI, T., et al., “Synthetic carbohydrate vaccines: synthesis and immunogenicity of Tn antigen conjugates”, Bioorg. Med. Chem., 11:1119-32 (1994).
15. WO1989/06974 by BIXLER, et al., “T-Cell Epitope As Carriers Molecule For Conjugate Vaccines” (1989-08-10).
16. WO1995/011998 by WANG, et al., “Structured Synthetic Antigen Libraries As Diagnostics, Vaccines And Therapeutics” (1995-05-04).
17. WO1999/066957 by WANG, “Artificial T Helper Cell Epitopes As Immune Stimulators For Synthetic Peptide Immunogens” (1999-12-29) and corresponding U.S. Pat. No. 6,713,301 (2004-03-30).
18. U.S. Pat. No. 5,912,176 by WANG, “Antibodies against a host cell antigen complex for pre and post exposure protection from infection by HIV” (1999-06-15) and related U.S. Pat. No. 5,961,976 (1999-10-05) and U.S. Pat. No. 6,090,388 (2000-07-18).
19. U.S. Pat. No. 6,025,468 by WANG, “Artificial T helper cell epitopes as immune stimulators for synthetic peptide immunogens including immunogenic LHRH peptides” (2000-02-15) and related U.S. Pat. No. 6,228,987 (2001-05-08) and U.S. Pat. No. 6,559,282 (2003-05-06).
20. U.S. Pat. No. 6,048,538 by WANG, et al., “Peptides derived from the non-structural proteins of foot and mouth disease virus as diagnostic reagents” (2000-04-11) and related U.S. Pat. No. 6,107,021 (2000-08-22).
21. U.S. Pat. No. 6,811,782 by WANG, et al., “Peptide composition as immunogen for the treatment of allergy” (2004-11-02) and related U.S. Pat. No. 7,648,701 (2010-01-19).
22. U.S. Pat. No. 6,906,169 by WANG, “Immunogenic peptide composition comprising measles virus Fprotein Thelper cell epitope (MUFThl-16) and N-terminus of β-amyloid peptide” (2005-06-14) and related U.S. Pat. No. 7,951,909 (2011-05-31) and U.S. Pat. No. 8,232,373 (2012-07-31).
23. U.S. Pat. No. 9,102,752 by WANG, “Peptide vaccine for prevention and immunotherapy of dementia of the Alzheimer's type” (2015-08-11).
24. US Publication No. 2013/0236487 by WANG, et al., “Designer Peptide-Based PCV2 Vaccine” (2013-09-12).
25. US Publication No. 2014/0335118 by WANG, “Synthetic Peptide-Based Marker Vaccine And Diagnostic System For Effective Control Of Porcine Reproductive And Respiratory Syndrome (PRRS)” (2014-11-13).
26. US Publication No. 2015/0306203 by WANG, “Synthetic Peptide-Based Emergency Vaccine Against Foot And Mouth Disease (FMD)” (2015-10-29).
27. US Publication No. 2017/0216418 by WANG, et al., “Immunogenic LHRH Composition And Use Thereof In Pigs” (2017-08-03).
28. International PCT Application No. PCT/US2017/069174 by WANG, “Peptide Immunogens and Formulations Thereof Targeting Membrane-Bound IgE for Treatment of IgE Mediated Allergic Diseases” (filed 2017 Dec. 31).
29. International PCT Application No. PCT/US2018/037938 by WANG, “Peptide Immunogens From the C-Terminal End of Alpha-Synuclein Protein and Formulations Thereof for Treatment of Synucleinopathies” (filed 2018 Jun. 15)
30. International PCT Application No. PCT/US2018/057840 by WANG, “Tau Peptide Immunogen Constructs” (filed 2018 Oct. 26).
31. International PCT Application No. PCT/US2018/065025 by WANG, “Peptide Immunogens of IL-31 and Formulations Thereof for the Treatment and/or Prevention of Atopic Dermatitis” (filed 2018 Dec. 11).

### BRIEF DESCRIPTION OF THE DRAWING

FIGS. 1A and 1B: Schematics showing an exemplary formulation and features of the T helper cell epitope platform described herein. FIG. 1A is a schematic summarizing components that can be included in formulations with peptide immunogens containing Th epitopes carriers, including UBITh®. FIG. 1B summarizes several features and technical advantages of the T helper cell epitope platform described herein, including UBITh®.

FIG. 2: A graph illustrating theoretical results that can be obtained using the T helper cell epitope platform described herein.

FIG. 3: Enhancement of the immunogenicity of peptide immunogen constructs by selected individual Th epitope peptides with short non-immunogenic B epitope peptides derived from alpha synuclein (top panel) and IgE EMPD (bottom panel). The numbers below the bar graph for alpha synuclein (top panel) correspond to the peptide immunogen constructs described in Table 5; and the numbers below the bar graph for IgE EMPD (bottom panel) correspond to the peptide immunogen constructs described in Table 6.

FIGS. 4A and 4B: Graphs illustrating anti-α-Synuclein antibody titers by ELISA obtained after immunizing guinea pigs with a mixture of three separate peptide immunogen constructs containing α-Syn (G111-G132), IgE-EMPD (G1-C39), and IL-6 (C73-C83) covalently linked to individual Th epitopes shown in Table 8. The specific α-Synuclein peptide immunogen constructs evaluated in these graphs are summarized in Table 5. FIG. 4A contains the antibody titer data for all of the peptide immunogen constructs evaluated. FIG. 4B contains a subset of the data shown in FIG. 4A to highlight that different peptide immunogen constructs are capable of reaching similar Cₘₐₓ values at different rates.

FIG. 5: A graph illustrating anti-IgE EMPD antibody titers by ELISA obtained after immunizing guinea pigs with a mixture of three separate peptide immunogen constructs containing α-Syn (G111-G132), IgE-EMPD (G1-C39), and IL-6 (C73-C83) covalently linked to individual Th epitopes shown in Table 8. The specific IgE EMPD peptide immunogen constructs evaluated in these graphs are summarized in Table 6.

FIG. 6: A graph illustrating anti-IL-6 antibody titers by ELISA obtained after immunizing guinea pigs with a mixture of three separate peptide immunogen constructs containing α-Syn (G111-G132), IgE-EMPD (G1-C39), and IL-6 (C73-C83) covalently linked to individual Th epitopes shown in Table 8. The specific IL-6 peptide immunogen constructs evaluated in these graphs are summarized in Table 7.

FIG. 7: Graphs illustrating the anti-DPR (di-peptide repeat) antibody titers by ELISA obtained after immunizing guinea pigs with the peptide immunogen constructs shown in Table 9.

FIG. 8: A graph illustrating the anti-Aβ₁₋₂₈ titers by ELISA obtained after immunizing guinea pigs with different amounts of the Aβ vaccine (UB-311), which contains two peptide immunogens Aβ₁₋₁₄-εK-KKK-MvF5 Th (SEQ ID NO: 67) and Aβ₁₋₁₄-εK-HBsAg3 Th (SEQ ID NO: 68).

FIG. 9: A graph illustrating the anti-Aβ₁₋₂₈ titers by ELISA obtained after immunizing guinea pigs with different prime and booster dosages of the Aβ vaccine (UB-311), which contains two peptide immunogens Aβ₁₋₁₄-εK-KKK-MvF5 Th (SEQ ID NO: 67) and Aβ₁₋₁₄-εK-HBsAg3 Th (SEQ ID NO: 68).

FIG. 10: Graphs illustrating the anti-Aβ₁₋₂₈ antibody titers by ELISA obtained after immunizing human subjects with the Aβ vaccine (UB-311) over a 3 month boosting regimen (top panel) or a 6 month boosting regimen (bottom panel) with the Aβ vaccine (UB-311). The boxed portion in each graph highlights the average titers for all human subjects in the study.

FIGS. 11A and 11B: Graphs illustrating the anti-LHRH antibody titers and testosterone concentration obtained after immunizing pigs with different amounts of a mixture of the LHRH peptide immunogen constructs shown in Table 10 (SEQ ID NOs: 239-241) using MONTANIDE ISA50V as an adjuvant. FIG. 11A shows the antibody titers and testosterone concentrations obtained after immunizing rats with 100 μg amount of the LHRH formulation. FIG. 11B shows the antibody titers and testosterone concentrations obtained after immunizing rats with 300 μg amount of the LHRH formulation.

FIG. 12: A graph illustrating the anti-IL-6 antibody titers by ELISA obtained after immunizing rats with different amounts the IL-6 peptide immunogen construct of SEQ ID NO: 243 or placebo controls in formulations containing different adjuvants (i.e., MONTANIDE ISA51 or ADJUPHOS).

FIGS. 13A and 13B: Graphs illustrating the anti-IgE-EMPD antibody titers obtained after immunizing macaques with different amounts of the IgE-EMPD peptide immunogen construct of SEQ ID NO: 178. FIG. 13A shows the antibody titers obtained using ADJUPHOS as an adjuvant formulated as a stabilized immunostimulatory complex using CpG3. FIG. 13B shows the antibody titers obtained using MONTANIDE ISA51 as an adjuvant formulated as a stabilized immunostimulatory complex using CpG3.

FIGS. 14A and 14B: Graphs illustrating the anti-LHRH antibody titers and testosterone concentration obtained after immunizing pigs with different amounts of a mixture of the LHRH peptide immunogen constructs shown in Table 10 (SEQ ID NOs: 239-241) using different adjuvants. FIG. 14A shows the antibody titers obtained using Emulsigen D as an adjuvant.

FIG. 14B shows the antibody titers obtained using MONTANIDE ISA50V as an adjuvant.

FIG. 15: Detection of promiscuous and artificial Th peptide responsive T cells in naïve Peripheral blood mononuclear cells of normal donors.

FIG. 16: Structures of tumor associated carbohydrate antigens (TACA): GD3, GD2, Globo-H, GM2, Fucosyl GM1, PSA, Le^{y}, Le^{x}, SLe^{x}, SLe^{a} and STn.

FIGS. 17 to 21: Illustrative Stepwise Synthesis via Solid Phase Peptide Synthesis Scheme of Glycan conjugated UBITh® peptide.

### DETAILED DESCRIPTION

The present disclosure provides promiscuous artificial T helper cell (Th) epitopes for stimulating functional site-directed antibodies against target antigen for preventative and therapeutic use. The present disclosure is also directed to peptide immunogen constructs that contain the Th epitopes, compositions containing the Th epitopes, methods of making and using the Th epitopes, and antibodies produced by peptide immunogen constructs containing the Th epitopes.

The disclosed artificial T helper cell (Th) epitopes can be linked to a B cell epitope and/or effector T cell epitope (e.g., cytotoxic T cell; CTL) (“target antigenic site(s)”) through an optional spacer to produce a peptide immunogen construct. The disclosed Th epitopes impart to the peptide immunogen the ability to induce a strong T helper cell-mediated immune response with the production of high level of antibodies and/or cellular responses (e.g., cytokine) against the target antigenic site for therapeutic effects. The disclosed peptide immunogen constructs provide for the advantageous replacement of large carrier proteins and pathogen-derived T helper cell sites in peptide immunogens with the disclosed artificial Th epitopes designed specifically to improve the immunogenicity of the target antigenic site. The relatively short peptide immunogen constructs containing the disclosed Th epitopes elicit a high level of antibodies and/or effector cell related cytokines to a specific target antigenic site without causing a significant inflammatory response or immune response against the Th epitope.

The immune response elicited by the peptide immunogen constructs (including antibody titers, Cₘₐₓ, onset of antibody production, duration of response, etc.) can be modulated by varying: (a) the choice of the Th epitope that is chemically linked to the B cell epitope, (b) the length of the B cell epitope, (c) the adjuvant that is used in the formulation containing the peptide immunogen construct, and/or (d) the dosing regimen including dosage per immunization and the prime and boost time points for each immunization. Therefore, specific immune responses to target antigenic sites can be designed using the disclosed Th epitopes, which can facilitate the tailoring of personalized medical treatment to the individual characteristics of any patient or subject.

The disclosed peptide immunogen constructs containing the artificial Th epitopes are capable of evoking antibody and/or cytokine responses in an immunized host against a desired target antigenic site. The target antigenic site can be a specific protein, a cancer antigen-related carbohydrate, a small molecule drug compound, or any amino acid sequence from any target peptide or protein. In some embodiments, the disclosure describes promiscuous artificial Th epitopes that can be used to provide peptide immunogens that elicit antibodies targeted to amyloid β (Aβ), foot-and-mouth disease (FMD) capsid protein, a glycoprotein from porcine reproductive and respiratory syndrome virus (PRRSV), Luteinizing Hormone-Releasing Hormone (LHRH), and any other peptide or protein sequence.

In certain embodiments, the target antigenic site is taken from a self-antigen or tumor-associated neoantigen target that is normally immunosilent (e.g., Aβ, Tau, Alpha Synuclein, Dipeptide protein, IgE EMPD, IL-6, CGRP, Amylin, IL-31, neoantigens, etc.). Non-limiting, representative sequences of self-antigens and tumor-associated neoantigen sites are shown in Table 3A. In other embodiments, the target antigenic site is taken from a pathogenic organism (e.g., FMDV, PRRSV, CSFV, HIV, HSV, etc.). Non-limiting, representative sequences of pathogenic antigenic sites are shown in Table 3B.

The peptides or target antigenic site of the invention can be useful in medical and veterinary applications. For example, the peptide immunogen constructs containing the disclosed artificial Th epitopes can be used in vaccine compositions to provide protective immunity from infectious diseases or neurodegenerative diseases, or pharmaceutical compositions to treat disorders resulting from malfunctioning normal physiological processes, immunotherapies for treating cancer, type 2 diabetes, or as agents to intervene in normal physiological processes.

### General

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Peptide Immunogen Constructs

The term “peptide immunogen” or “peptide immunogen construct” as used herein refers to molecules comprising artificial Th epitopes covalently linked to a target antigenic site, with or without a heterologous spacer, through covalent linkages (e.g., a conventional peptide bond or a thioester) so as to form a single larger peptide. Typically, peptide immunogen constructs contain (a) a heterologous promiscuous artificial Th epitope; (b) a target antigenic site such as a B cell epitope or effector T cell epitope (e.g., CTL); and (c) an optional heterologous spacer.

The presence of a promiscuous artificial Th epitope in a peptide immunogen can induce a strong Th cell-mediated immune response and high level of antibodies directed to a target antigenic site in an animal after immunization with the peptide immunogen. The disclosed peptide immunogen constructs provide for the advantageous replacement of large carrier proteins and pathogen-derived T helper cell sites in peptide immunogens with the disclosed artificial Th epitopes designed specifically to improve the immunogenicity of the target antigenic site. The relatively short peptide immunogen constructs containing the disclosed Th epitopes elicit a high level of antibodies and/or effector cell related cytokines to a specific target antigenic site without causing a significant inflammatory response or immune response against the Th epitope.

Peptide immunogen constructs containing the disclosed artificial Th epitopes of the present invention can be represented by the following formulae:

(A)ₙ-(Target antigenic site)-(B)ₒ-(Th)ₘ-(A)ₙ-X

or

(A)ₙ-(Th)ₘ-(B)ₒ-(Target antigenic site)-(A)ₙ-X

or

(A)ₙ-(Th)ₘ-(B)ₒ-(Target antigenic site)-(B)ₒ-(Th)ₘ-(A)ₙ-X

or

{(A)ₙ-(Th)ₚ-(B)ₒ-(Target antigenic site)-(B)ₒ-(Th)ₚ-(A)ₙ-X}m

wherein:
each A is independently an amino acid;
each B is independently a heterologous spacer;
each Th is independently an artificial Th epitope;
Target antigenic site is a B cell epitope, a CTL epitope, a peptide hapten, a non-peptide hapten, or an immunologically reactive analogue thereof;
X is an amino acid, α-COOH, or α-CONH₂;
n is 0, 1, 2, 3, 4, 5, 6, 7, 8, 9, or 10;
m is 1, 2, 3, or 4;
o is 0, 1, 2, 3, 4, 5, 6, 7, 8, 9, or 10; and
p is 0, 1, 2, 3, or 4.

The peptide immunogens of the disclosure can comprise between about 20 to about 100 amino acids. In some embodiments, the peptide immunogen construct contains about 20, about 25, about 30, about 35, about 40, about 45, about 50, about 55, about 60, about 65, about 70, about 75, about 80, about 85, about 90, about 95, or about 100 amino acid residues.

The various components of the disclosed IL-31 peptide immunogen construct are described below.

### A—Amino Acid

Each A in the immunogenic peptides of the disclosure is independently a heterologous amino acid.

The term “heterologous”, as used herein, refers to an amino acid sequence that is not part of, or homologous with, the wild-type amino acid sequence of the target antigenic site (e.g., B cell epitope). Thus, a heterologous amino acid sequence of A contains an amino acid sequence that is not naturally found in the protein or peptide of the target antigenic site. Since the sequence of component A is heterologous to the target antigenic site, the natural amino acid sequence of target antigenic site is not extended in either the N-terminal or C-terminal directions when component A is covalently linked to the target antigenic site.

In some embodiments, each A is independently a non-naturally occurring or naturally occurring amino acid.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

In some embodiments, n is 0 indicating that no amino acid is added at that position in the formula. In other embodiments, n is 1 and selected from any natural or non-natural amino acid. In certain embodiments, n is greater than one, and each A is independently the same amino acid. In other embodiments, n is greater than 1 and each A is independently a different amino acid.

### B—Optional Heterologous Spacer

Each B in the immunogenic peptide of the disclosure is an optional heterologous spacer. The optional heterologous spacer of component B is independently an amino acid, —NHCH(X)CH₂SCH₂CO—, —NHCH(X)CH₂SCH₂CO(εN)Lys-, —NHCH(X)CH₂S-succinimidyl(εN)Lys-, —NHCH(X)CH₂S-(succinimidyl)-, and/or any combination thereof. The spacer can contain one or more naturally or non-naturally occurring amino acid residues as described above for component A.

As discussed above, term “heterologous” refers to an amino acid that is not part of, or homologous with, the wild-type amino acid sequence of the target antigenic site (e.g., B cell epitope). Thus, when the spacer is an amino acid, the spacer contains an amino acid sequence that is not naturally found in the protein or peptide of the target antigenic site. Since the sequence of component B is heterologous to the target antigenic site, the natural amino acid sequence of target antigenic site is not extended in either the N-terminal or C-terminal directions when component B is covalently linked to the target antigenic site.

The spacer can be a flexible hinge spacer to enhance the separation of a Th epitope and the target antigenic site. In some embodiments, a flexible hinge sequence can be proline rich. In certain embodiments, the flexible hinge has the sequence Pro-Pro-Xaa-Pro-Xaa-Pro (SEQ ID NO: 55), which is modeled from the flexible hinge region found in immunoglobulin heavy chains. Xaa therein can be any amino acid. In some embodiments, Xaa is aspartic acid. In some embodiments, the conformational separation provided by a spacer can permit more efficient interactions between a presented peptide immunogen and appropriate Th cells and B cells. Immune responses to the Th epitope can be enhanced to provide improved immune reactivity.

When o>1, each B is independently the same or different. In some embodiments, B is Gly-Gly, Pro-Pro-Xaa-Pro-Xaa-Pro (SEQ ID NO: 55), εNLys, εNLys-Lys-Lys-Lys (SEQ ID NO: 53), Lys-Lys-Lys-εNLys (SEQ ID NO: 54), Lys-Lys-Lys, —NHCH(X)CH₂SCH₂CO—, —NHCH(X)CH₂SCH₂CO(εNLys)-, —NHCH(X)CH₂S-succinimidyl-εNLys-, or —NHCH(X)CH₂S-(succinimidyl)-, and/or any combination thereof. Exemplary heterologous spacers are shown in Table 2.

### Target Antigenic Site

The target antigenic site can include any amino acid sequence from any target peptide or protein, including foreign- or self-peptides or proteins, a B cell epitope, a CTL epitope, a peptide hapten, a non-peptide hapten, or an immunologically reactive analogue thereof. The target antigenic site can be a specific protein, a cancer antigen-related carbohydrate, a small molecule drug compound, or any amino acid sequence from any target peptide or protein. In some embodiments, the disclosure describes promiscuous artificial Th epitopes that can be used to provide peptide immunogens that elicit antibodies targeted to amyloid β (Aβ), foot-and-mouth disease (FMD) capsid protein, a glycoprotein from porcine reproductive and respiratory syndrome virus (PRRSV), Luteinizing Hormone-Releasing Hormone (LHRH), and any other peptide or protein sequence.

In certain embodiments, the target antigenic site is taken from a self-antigen or tumor-associated neoantigen target that is normally immunosilent (e.g., Aβ, Tau, Alpha Synuclein, Dipeptide protein, IgE EMPD, IL-6, CGRP, Amylin, IL-31, neoantigens, etc.). Non-limiting, representative sequences of self-antigens and tumor-associated neoantigen sites are shown in Table 3A. In other embodiments, the target antigenic site is taken from a pathogenic organism (e.g., FMDV, PRRSV, CSFV, HIV, HSV etc.). Non-limiting, representative sequences of pathogenic antigenic sites are shown in Table 3B.

In specific embodiments, the target antigenic site is derived from portions of luteinizing hormone-releasing hormone (LHRH) (e.g., U.S. Pat. Nos. 6,025,468, 6,228,987, 6,559,282, and US Publication No. US2017/0216418); amyloid R (Aβ) (e.g., U.S. Pat. Nos. 6,906,169, 7,951,909, 8,232,373, and 9,102,752); foot-and-mouth disease capsid protein (e.g., U.S. Pat. Nos. 6,048,538, 6,107,021, and US Publication No. 2015/0306203); HIV virion epitopes for prevention and treatment of HIV infection (e.g., U.S. Pat. Nos. 5,912,176, 5,961,976, and 6,090,388); a capsid protein from porcine circovirus type 2 (PCV2) (e.g., US Publication No. 2013/0236487), a glycoprotein from porcine reproductive and respiratory syndrome virus (PRRSV) (e.g., US Publication No. 2014/0335118), IgE (e.g., U.S. Pat. Nos. 7,648,701 and 6,811,782), alpha-synuclein (α-Syn) (International PCT Application No. PCT/US2018/037938), the extracellular membrane-proximal domain of membrane-bound IgE (or IgE EMPD) (International PCT Application No. PCT/US2017/069174), Tau (International PCT Application No. PCT/US2018/057840), and Interleukin-31 (IL-31) (International PCT Application No. PCT/US2018/065025), the CS antigen of plasmodium for prevention of malaria; CETP for prevention and treatment of arteriosclerosis; IAPP (Amylin) for the prevention and treatment of type 2 diabetes, and any other peptide or protein sequence. All of the patents and patent publications are herein incorporated by references in their entireties.

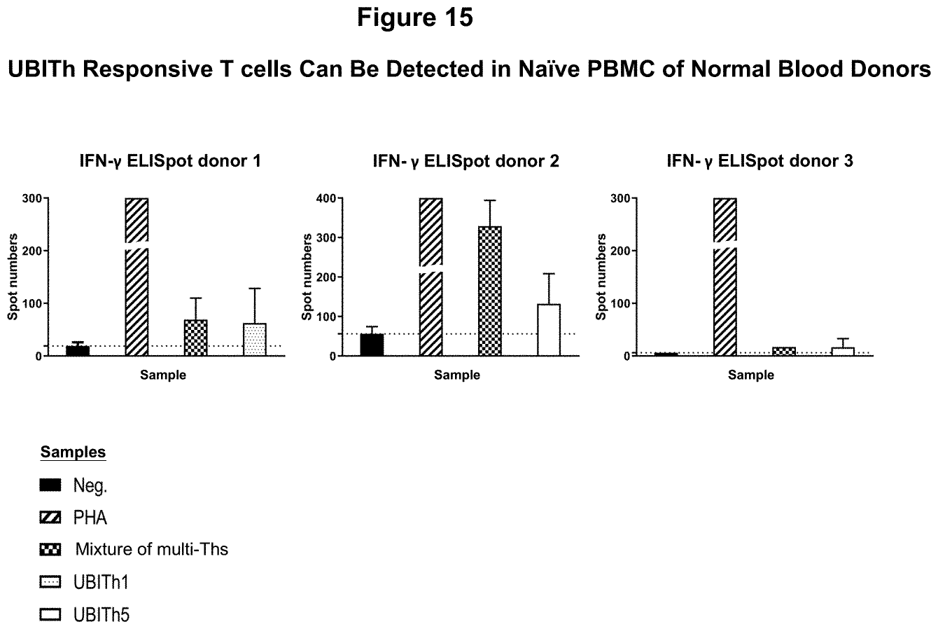

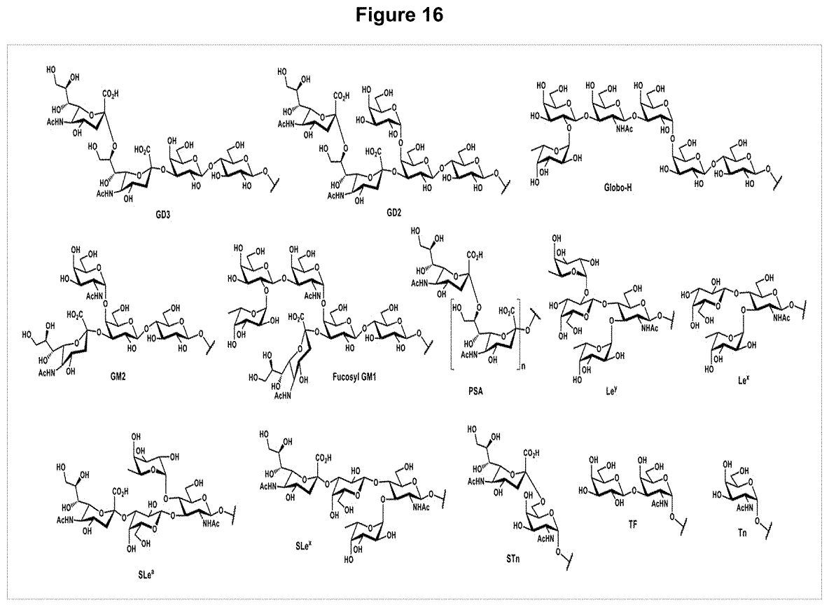

In other embodiments, the target antigenic site is a non-peptide hapten, including tumor associated carbohydrate antigens (TACA) and small-molecule drug compound. Examples of TACAs include GD3, GD2, Globo-H, GM2, Fucosyl GM1, GM2, PSA, Le^{y}, Le^{x}, SLe^{x}, SLe^{a}, Tn, TF, and STn, as discussed further in Example 11 and FIGS. 16 and 17.

### Th—T Helper Epitope

The promiscuous artificial T helper cell (Th) epitope in the peptide immunogen construct enhances the immunogenicity of the target antigenic site, which facilitates the production of specific high titer antibodies directed against the optimized target B cell epitope through rational design.

The term “promiscuous”, as used herein, refers to a Th epitope that is reactive across species and across individuals of a single species.

The term “artificial”, as used in connection with the Th epitopes, refers to amino acid sequences that are not found in nature. Accordingly, the artificial Th epitopes of the present disclosure have heterologous sequences to the target antigenic site. As discussed above, the term “heterologous” refers to an amino acid sequence that is derived from an amino acid sequence that is not part of, or homologous with, the wild-type sequence of the target antigenic site. Thus, a heterologous Th epitope is a Th epitope derived from an amino acid sequence that is not naturally found in the target antigenic site. Since the Th epitope is heterologous to the target antigenic site, the natural amino acid sequence of the target antigenic site is not extended in either the N-terminal or C-terminal directions when the heterologous Th epitope is covalently linked to the target antigenic site.

The Th epitope can have an amino acid sequence derived from any species (e.g., human, pig, cattle, dog, rat, mouse, guinea pigs, etc.). The Th epitope can also have promiscuous binding motifs to NMC class II molecules of multiple species. In certain embodiments, the Th epitope comprises multiple promiscuous NMC class II binding motifs to allow maximal activation of T helper cells leading to initiation and regulation of immune responses. The Th epitope is preferably immunosilent on its own, i.e., little, if any, of the antibodies generated by the peptide immunogen constructs will be directed towards the Th epitope, thus allowing a very focused immune response directed to the targeted antigenic site.

Th epitopes can range in size from approximately 15 to approximately 50 amino acid residues. In some embodiments, Th epitopes can have about 15, about 20, about 25, about 30, about 35, about 40, about 45, or about 50 amino acid residues. Th epitopes can share common structural features and specific landmark sequences. In some embodiments, Th epitopes have amphipathic helices, i.e., alpha-helical structures with hydrophobic amino acid residues dominating one face of the helix and charged and polar resides dominating the surrounding faces.

The Th epitopes and disclosures of WO 1999/066957, and corresponding U.S. Pat. No. 6,713,301, are incorporated herein by reference in their entireties.

A promiscuous Th determinant can be effective in potentiating a poorly immunogenic peptide. Well-designed promiscuous Th/B cell epitope chimeric peptides can elicit Th responses with antibody responses targeted to the B cell site in most members of a genetically diverse population. In some embodiments, Th cells can be supplied to a target antigen peptide by covalently binding a peptide-carrier to a well-characterized promiscuous Th determinant.

Promiscuous Th epitopes can contain additional primary amino acid patterns. In some embodiments, promiscuous Th epitopes can contain a Rothbard sequence, wherein the promiscuous Th epitope contains a charged residue (e.g., -Gly-), followed by two to three hydrophobic residues, followed by a charged or polar residue (Rothbard and Taylor, EMBO J, 1988; 7:93-101). Promiscuous Th epitopes can obey the 1, 4, 5, 8 rule, wherein a positively charged residue is followed by hydrophobic residues at the fourth, fifth and eighth positions, consistent with an amphipathic helix having positions 1, 4, 5 and 8 located on the same face. In some embodiments, the 1, 4, 5, 8 pattern of hydrophobic and charged and polar amino acids can be repeated within a single Th epitope. In some embodiments, a promiscuous T cell epitope can contain at least one of a Rothbard sequence or an epitope that obeys the 1, 4, 5, 8 rule. In other embodiments, the Th epitope contains more than one Rothbard sequence.

Promiscuous Th epitopes derived from pathogens include, but are not limited to: a hepatitis B surface Th cell epitope (HBsAg Th), hepatitis B core antigen Th cell epitope (HBc Th), pertussis toxin Th cell epitope (PT Th), tetanus toxin Th cell epitope (TT Th), measles virus F protein Th cell epitope (MVF Th), Chlamydia trachomatis major outer membrane protein Th cell epitope (CT Th), diphtheria toxin Th cell epitope (DT Th), Plasmodium falciparum: circumsporozoite Th cell epitope (PF Th), Schistosoma mansoni triose phosphate isomerase Th cell epitope (SM Th), and a Escherichia coli TraT Th cell epitope (TraT Th), Clostridium tetani, Bordetella pertussis, Cholera Toxin, Influenza MP1, Influenza NSP1, Epstein Barr virus (EBV), Human cytomegalovirus (HCMV). Examples of Th epitopes used in the present disclosure are shown in Table 1.

In some embodiments, the Th epitopes of the disclosure can be combinatorial Th epitopes containing a mixture of peptides containing similar amino acid sequences. Structured synthetic antigen libraries (SSALs), also referred to as combinatorial artificial Th epitopes, comprise a multitude of Th epitopes with amino acid sequences organized around a structural framework of invariant residues with substitutions at specific positions. The sequences of SSAL epitopes are determined by retaining relatively invariant residues and varying other residues to provide recognition of the diverse MHC restriction elements. Sequences of SSAL epitopes can be determined by aligning the primary amino acid sequence of a promiscuous Th, selecting and retaining residues responsible for the unique structure of the Th peptide as the skeletal framework, and varying the remaining residues in accordance with known MHC restriction elements. Invariant and variable positions with preferred amino acids of MHC restriction elements can be used to obtain MHC-binding motifs, which can be used to design a SSAL of Th epitopes.

The heterologous Th epitope peptides presented as a combinatorial sequence, contain a mixture of amino acid residues represented at specific positions within the peptide framework based on the variable residues of homologues for that particular peptide. In some embodiments, the Th epitope library sequences are designed to maintain the structural motifs of a promiscuous Th epitope and to accommodate reactivity to a wider range of haplotypes. In some embodiments, a member of a SSAL can be the degenerate Th epitope SSAL1 Th1, modeled after a promiscuous epitope taken from the F protein of the measles virus (e.g., SEQ ID NOs: 1-5). In other embodiments, a member of a SSAL can be the degenerate Th epitope SSAL2 Th2, modeled after a promiscuous epitope taken from HBsAg1 (e.g., SEQ ID NOs: 19-24).

The total number of peptides present in a mixture of combinatorial artificial Th epitopes (or SSAL) after synthesis can be calculated by multiplying the number of options available at each variable position together. For example, SEQ ID NO: 16 represents a combination of 32 different peptides because it contains 5 variable positions, where each variable position has an option of 2 different residues (i.e., 2×2×2×2×2=25=32). Similarly, SEQ ID NO: 5 represents a combination of 524,288 different peptides (i.e., 2×4×2×4×2×4×4×4×2×4×2×4=25×47=524,288). The combinatorial artificial Th epitope sequences include (a) the mixture of all the peptides encompassed by the variable sequences and (b) each individual peptide containing a single-sequence within the combination.

In some embodiments, a charged residue Glu or Asp can be added at position 1 to increase the charge surrounding the hydrophobic face of the Th. In some embodiments, the hydrophobic face of an amphipathic helix can be maintained by hydrophobic residues at 2, 5, 8, 9, 10, 13 and 16. In some embodiments, amino acid residues at 2, 5, 8, 9, 10, and 13 can be varied to provide a facade with the capability of binding to a wide range of MHC restriction elements. In some embodiments, variation in amino acid residues can enlarge the range of immune responsiveness of the artificial Th epitopes.

Artificial Th epitopes can incorporate all properties and features of known promiscuous Th epitopes. In some embodiments, the artificial Th epitopes are members of an SSAL. In some embodiments, an artificial Th site can be combined with peptide sequences taken from self-antigens and foreign antigens to provide enhanced antibody responses to site-specific targets. In some embodiments, an artificial Th epitope immunogen can provide effective and safe antibody responses, exhibit high immunopotency, and demonstrate broad reactive responsiveness.

Idealized artificial Th epitopes are also provided. These idealized artificial Th epitopes are modeled on two known natural Th epitopes and SSAL peptide prototypes, disclosed in WO 95/11998. The SSALS incorporate combinatorial MHC molecule binding motifs (Meister et al., 1995) intended to elicit broad immune responses among the members of a genetically diverse population. The SSAL peptide prototypes were designed based on the Th epitopes of the measles virus and hepatitis B virus antigens, modified by introducing multiple MHC-binding motifs. The design of the other Th epitopes were modeled after other known Th epitopes by simplifying, adding, and/or modifying, multiple MHC-binding motifs to produce a series of novel artificial Th epitopes. The promiscuous artificial Th sites were incorporated into synthetic peptide immunogens bearing a variety of target antigenic sites. The resulting chimeric peptides were able to stimulate effective antibody responses to the target antigenic sites.

The prototype artificial helper T cell (Th) epitope shown in Table 1 as “SSAL1 Th1”, a mixture of four peptides (SEQ ID NOs: 1-4) is an idealized Th epitope modeled from a promiscuous Th epitope of the F protein of measles virus (Partidos et al. 1991). The model Th epitope, shown in Table 1 as “MVF Th (UBITh®5)” (SEQ ID NO: 6) corresponds to residues 288-302 of the measles virus F protein. MVF Th (SEQ ID NO: 6) was modified to the SSAL1 Th1 prototype (SEQ ID NOs: 1-4) by adding a charged residue Glu/Asp at position 1 to increase the charge surrounding the hydrophobic face of the epitope; adding or retaining a charged residues or Gly at positions 4, 6, 12 and 14; and adding or retaining a charged residue or Gly at positions 7 and 11 in accordance with the “Rothbard Rule”. The hydrophobic face of the Th epitope comprise residues at positions 2, 5, 8, 9, 10, 13, and 16. Hydrophobic residues commonly associated with promiscuous epitopes were substituted at these positions to provide the combinatorial Th SSAL epitopes, SSAL1 Th1 (SEQ ID NOs: 1-4). Another significant feature of the prototype SSAL1 Th1 (SEQ ID NOs: 1-4) is that positions 1 and 4 is imperfectly repeated as a palindrome on either side of position 9, to mimic an MHC-binding motif. This “1, 4, 9” palindromic pattern of SSAL1 Th1 was further modified in SEQ ID NO: 2 (Table 1) to more closely reflect the sequence of the original MvF model Th (SEQ ID NO: 6).

Combinatorial artificial Th epitopes can be simplified to provide a series of single-sequence epitopes. For example, the combinatorial sequence of SEQ ID NO: 5 can be simplified to the single sequence Th epitopes represented by SEQ ID NOs: 1-4. These single sequence Th epitopes can be coupled to target antigenic sites to provide enhanced immunogenicity.

In some embodiments, the immunogenicity of the Th epitopes may be improved by extending the N terminus with a non-polar and a polar uncharged amino acid, e.g., Ile and Ser, and extending the C terminus by a charged and hydrophobic amino acid, e.g., Lys and Phe. In addition, the addition of a Lysine residue or multiple lysine residues (e.g., KKK) to the Th epitopes can improve the solubility of the peptide in water. Further modifications included the substitution of the C-termini by a common MHC-binding motif AxTxIL (Meister et al, 1995).

An artificial Th epitope can be a known natural Th epitope or an SSAL peptide prototype.

In some embodiments, a Th epitope from an SSAL can incorporate combinatorial MHC molecule binding motifs intended to elicit broad immune responses among the members of a genetically diverse population. In some embodiments, a SSAL peptide prototype can be designed based on Th epitopes of the measles virus and hepatitis B virus antigens, modified by introducing multiple MHC-binding motifs. In some embodiments, an artificial Th epitope can simplify, add, or and/or modify multiple MHC-binding motifs to produce a series of novel artificial Th epitopes. In some embodiments, newly adapted promiscuous artificial Th sites can be incorporated into synthetic peptide immunogens bearing a variety of target antigenic sites. In some embodiments, resulting chimeric peptides can stimulate effective antibody responses to target antigenic sites.

Artificial Th epitopes of the disclosure can be contiguous sequences of natural or non-natural amino acids that comprise a class II MHC molecule binding site. In some embodiments, an artificial Th epitope can enhance or stimulate an antibody response to a target antigenic site. In some embodiments, a Th epitope can consist of continuous or discontinuous amino acid segments. In some embodiments, not every amino acid of a Th epitope is involved with MHC recognition. In some embodiments, the Th epitopes of the invention can comprise immunologically functional homologues, such as immune-enhancing homologues, cross reactive homologues, and segments thereof. In some embodiments, functional Th homologues can further comprise conservative substitutions, additions, deletions, and insertions of 1, 2, 3, 4, 5, 6, 7, 8, 9, or 10 amino acid residues and provide the Th-stimulating function of a Th epitope.

Th epitopes can be attached directly to the target site. In some embodiments, the Th epitopes can be attached to the target site through an optional heterologous spacer, e.g., a peptide spacer such as Gly-Gly or (ε-N)Lys. The spacer physically separates the Th epitope from the B cell epitope, and can disrupt the formation of any artificial secondary structures created by the linking of the Th epitope or a functional homologue with the target antigenic site, thereby eliminating any interference with the Th and/or B cell responses.

Th epitopes include idealized artificial Th epitopes and combinatorial idealized artificial Th epitopes, as shown in Table 1. In some embodiments, the Th epitope is a promiscuous Th cell epitope of SEQ ID NOs: 1-52, any homologue thereof, and/or any immunological analogue thereof. Th epitopes also include immunological analogues of Th epitopes. Immunological Th analogues include immune-enhancing analogs, cross-reactive analogues and segments of any of these Th epitopes that are sufficient to enhance or stimulate an immune response to the target antigenic site.

Functional immunologically analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the target antigenic site. Table 1 identifies another variation of a functional analogue for Th epitope peptide. In particular, SEQ ID NOs: 6 and 7 of MvF1 and MvF2 Th are functional analogues of SEQ ID NOs: 16 and 17 of MvF4 and MvF5 in that they differ in the amino acid frame by the deletion (SEQ ID NOs: 6 and 7) or the inclusion (SEQ ID NOs: 16 and 17) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 6-18) and from Hepatitis Surface protein HBsAg 1-3 Ths (SEQ ID NOs: 19-31).

The Th epitope in peptide immunogen construct can be covalently linked at either N- or C-terminal end of the target antigenic site to produce a chimeric Th/B cell site peptide immunogen. In some embodiments, a Th epitope can be covalently attached to the target antigenic site via chemical coupling or via direct synthesis. In some embodiments, the Th epitope is covalently linked to the N-terminal end of the target antigenic site. In other embodiments, the Th epitope is covalently linked to the C-terminal end of the target antigenic site. In certain embodiments, more than one Th epitope is covalently linked to the target antigenic site. When more than one Th epitope is linked to the target antigenic site, each Th epitope can have the same amino acid sequence or different amino acid sequences. In addition, when more than one Th epitope is linked to the target antigenic site, the Th epitopes can be arranged in any order. For example, the Th epitopes can be consecutively linked to the N-terminal end of the target antigenic site, or consecutively linked to the C-terminal end of the target antigenic site, or a Th epitope can be covalently linked to the N-terminal end of the target antigenic site while a separate Th epitope is covalently linked to the C-terminal end of the target antigenic site. There is no limitation in the arrangement of the Th epitopes in relation to the target antigenic site.

In some embodiments, the Th epitope is covalently linked to the target antigenic site directly. In other embodiments, the Th epitope is covalently linked to the target antigenic site through a heterologous spacer described in further detail below.

### Methods of Synthesis

The peptide immunogens of the disclosure can be synthesized using chemical methods. In some embodiments, the peptide immunogens of the disclosure can be synthesized using solid phase peptide synthesis. In some embodiments, the peptides of the invention are synthesized using automated Merrifield solid phase peptide synthesis using t-Boc or Fmoc to protect α-NH₂ or side chain amino acids.

The heterologous Th epitope peptides presented as a combinatorial sequence contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 5, 10, 13, 16, 24, and 27 which are shown in Table 1. Th epitope peptides of the present invention provide broad reactivity and immunogenicity to animals and patients from genetically diverse populations.

Interestingly, inconsistencies and/or errors that might be introduced during the synthesis of the Th epitope, B cell epitope, and/or the peptide immunogen construct containing a Th epitope and B cell epitope most often do not hinder or prevent a desired immune response in a treated animal. In fact, inconsistencies/errors that might be introduced during the peptide synthesis generate multiple peptide analogues along with the targeted peptide syntheses. These analogues can include amino acid insertion, deletion, substitution, and premature termination. As described above, such peptide analogues are suitable in peptide preparations as contributors to antigenicity and immunogenicity when used in immunological application either as solid phase antigen for purpose of immunodiagnosis or as immunogens for purpose of vaccination.

Peptide immunogen constructs comprising Th epitopes are produced simultaneously in a single solid-phase peptide synthesis in tandem with the target antigenic site. Th epitopes also include immunological analogues of Th epitopes. Immunological Th analogues include immune-enhancing analogs, cross-reactive analogues and segments of any of these Th epitopes that are sufficient to enhance or stimulate an immune response to the target antigenic site.

After the complete assembly of a desired peptide immunogen, the solid phase resin can be treated to cleave the peptide from the resin and to remove the functional groups on the amino acid side chains. The free peptide can be purified by HPLC and characterized biochemically. In some embodiments, the free peptides are characterized biochemically using amino acid analysis. In some embodiments, the free peptides are characterized using peptide sequence. In some embodiments, the free peptides are characterized using mass spectrometry.

The peptide immunogens of the invention can be synthesized using haloacetylated and cysteinylated peptides through the formation of a thioether linkage. In some embodiments, a cysteine can be added to the C terminus of a Th-containing peptide, and the thiol group of the cysteine residue can be used to form a covalent bond to an electrophilic group such as a Na chloroacetyl-modified group or a maleimide-derivatized α- or ε-NH₂ group of a lysine residue. The resulting synthetic intermediate can be attached to the N-terminus of a target antigenic site peptide.

Longer synthetic peptide conjugates can be synthesized using nucleic acid cloning techniques. In some embodiments, the Th epitopes of the invention can be synthesized by expressing recombinant DNA and RNA. To construct a gene expressing a Th/target antigenic site peptide of this invention, an amino acid sequence can be reverse translated into a nucleic acid sequence. In some embodiments, an amino acid sequence is reverse translated into a nucleic acid sequence using optimized codons for the organism in which the gene will be expressed. A gene encoding the peptide can be made. In some embodiments, a gene encoding a peptide can be made by synthesizing overlapping oligonucleotides that encode the peptide and necessary regulatory elements. The synthetic gene can be assembled and inserted into a desired expression vector.

The synthetic nucleic acid sequences of the disclosure can include nucleic acid sequences that encode Th epitopes of the invention, peptides comprising Th epitopes, immunologically functional homologues thereof, and nucleic acid constructs characterized by changes in the non-coding sequences that do not alter the immunogenic properties of the peptide or encoded Th epitope. The synthetic gene can be inserted into a suitable cloning vector, and recombinants can be obtained and characterized. The Th epitopes and peptides comprising the Th epitopes can then be expressed under conditions appropriate for a selected expression system and host. The Th epitope or peptide can be purified and characterized.

### Pharmaceutical Compositions

The present disclosure also describes pharmaceutical compositions comprising peptide immunogens of the disclosure. In some embodiments, a pharmaceutical composition of the disclosure can be used as a pharmaceutically acceptable delivery system for the administration of peptide immunogens. In some embodiments, a pharmaceutical composition of the disclosure can comprise an immunologically effective amount of one or more of the peptide immunogens.

The peptide immunogens of the invention can be formulated as immunogenic compositions. In some embodiments, an immunogenic composition can comprise adjuvants, emulsifiers, pharmaceutically-acceptable carriers or other ingredients routinely provided in vaccine compositions. Adjuvants or emulsifiers that can be used in this invention include alum, incomplete Freund's adjuvant (IFA), liposyn, saponin, squalene, L121, emulsigen, monophosphoryl lipid A (MPL), dimethyldioctadecylammonium bromide (DDA), QS21, and ISA 720, ISA 51, ISA 35, ISA 206, and other efficacious adjuvants and emulsifiers. In some embodiments, a composition of the invention can be formulated for immediate release. In some embodiments, a composition of the invention can be formulated for sustained release.

Adjuvants used in the pharmaceutical composition can include oils, aluminum salts, virosomes, aluminum phosphate (e.g., ADJU-PHOS®), aluminum hydroxide (e.g., ALHYDROGEL®), liposyn, saponin, squalene, L121, Emulsigen®, monophosphoryl lipid A (MPL), QS21, ISA 35, ISA 206, ISA50V, ISA51, ISA 720, as well as the other adjuvants and emulsifiers.

In some embodiments, the pharmaceutical composition contains MONTANIDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e., w/o/w) emulsion with EMULSIGEN or EMULSIGEN D as the adjuvant.

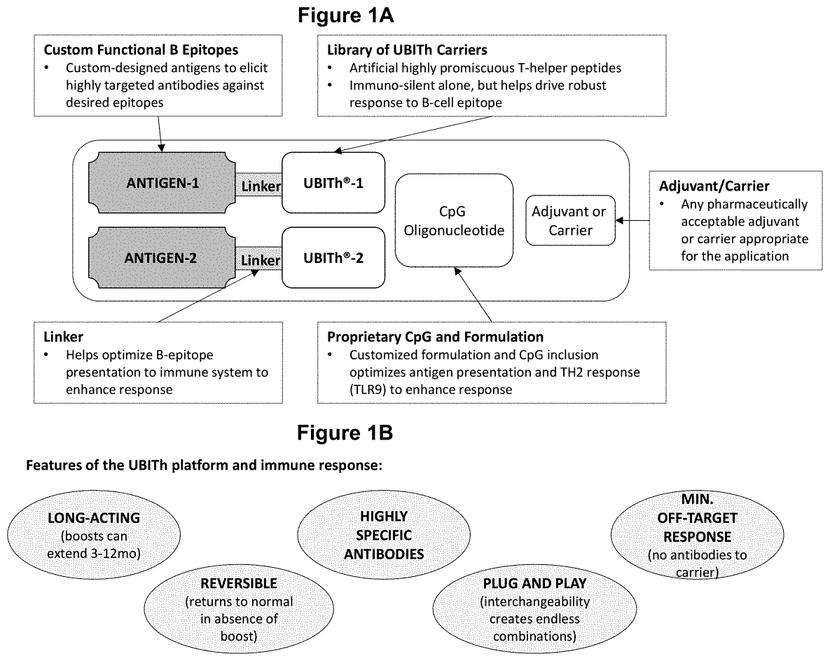

FIG. 1A is a schematic summarizing components that can be included in formulations with peptide immunogens containing Th epitopes carriers, including UBITh®. The exemplary formulation shown in FIG. 1A contains two separate peptide immunogen constructs. Each peptide immunogen construct contains (a) a custom functional B cell epitope that is designed to elicit highly targeted antibodies against desired epitopes, (b) a linker that helps optimize B cell epitope presentation to the immune system to enhance immunogenicity, and (c) a Th epitope that is immunosilent on its own, but helps drive robust response to the B cell epitope. The formulation also contains a pharmaceutically acceptable adjuvant or carrier that is appropriate for the particular application in which the composition will be used. Furthermore, the composition can be formulated into a stabilized immunostimulatory complex using a CpG oligonucleotide (described further below).

FIG. 1B summarizes several features and technical advantages of the T helper cell epitope platform described herein, including UBITh®. For example, the Th epitope platform described herein is long-acting, but is reversible in the absence of a boost. The Th epitope platform generates highly specific antibodies directed to the B cell epitope with little, if any, antibodies being generated toward the linker or Th epitope sequences. Furthermore, the Th epitope platform can be used with a wide variety of B cell epitopes, which allows endless combinations of peptide immunogen constructs.

In some embodiments, a composition is formulated for use as a vaccine. A vaccine composition can be administered by any convenient route, including subcutaneous, oral, intramuscular, intraperitoneal, parenteral, or enteral administration. In some embodiments, the immunogens are administered in a single dose. In some embodiments, immunogens are administered over multiple doses.

Pharmaceutical compositions can be prepared as injectables, either as liquid solutions or suspensions. Liquid vehicles containing the tau peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

The composition of the instant invention can contain an effective amount of one or more peptide immunogens and a pharmaceutically acceptable carrier. In some embodiments, a composition in a suitable dosage unit form can contain about 0.5 μg to about 1 mg of a peptide immunogen per kg body weight of a subject. In some embodiments, a composition in a suitable dosage unit form can contain about 10 μg, about 20 μg, about 30 μg, about 40 μg, about 50 μg, about 60 μg, about 70 μg, about 80 μg, about 90 μg, about 100 μg, about 200 μg, about 300 μg, about 400 μg, about 500 μg, about 600 μg, about 700 μg, about 800 μg, about 900 μg, or about 1000 μg of a peptide immunogen per kg body weight of a subject. In some embodiments, a composition in a suitable dosage form can contain about 100 μg, about 150 μg, about 200 μg, about 250 μg, about 300 μg, about 350 μg, about 400 μg, about 450 μg, or about 500 μg of a peptide immunogen per kg body weight of a subject. In some embodiments, a composition in a suitable dosage unit form can contain about 0.5 μg to about 1 mg of a peptide immunogen per kg body weight of a subject. In some embodiments, a composition in a suitable dosage unit form can contain about 10 μg, about 20 μg, about 30 μg, about 40 μg, about 50 μg, about 60 μg, about 70 μg, about 80 μg, about 90 μg, about 100 μg, about 200 μg, about 300 μg, about 400 μg, about 500 μg, about 600 μg, about 700 μg, about 800 μg, about 900 μg, or about 1000 μg of a peptide immunogen. In some embodiments, a composition in a suitable dosage form can contain about 100 μg, about 150 μg, about 200 μg, about 250 μg, about 300 μg, about 350 μg, about 400 μg, about 450 μg, or about 500 μg of a peptide immunogen.

When delivered in multiple doses, a composition can be divided into an appropriate amount per dose. In some embodiments, a dose is about 0.2 mg to about 2.5 mg. In some embodiments, a dose is about 1 mg. In some embodiments, a dose is about 1 mg and is administered by injection. In some embodiments, a dose is about 1 mg and is administered intramuscularly. In some embodiments, a dose can be followed by a repeat (booster) dose. Dosages can be optimized depending on the age, weight, and general health of the subject.

Vaccines comprising mixtures of peptide immunogens can provide enhanced immunoefficacy in a broader population. In some embodiments, a mixture of peptide immunogens comprises Th sites derived from MVF Th and HBsAg Th. In some embodiments, vaccines comprising mixtures of peptide immunogens can provide an improved immune response to the target antigenic site.

The immune response to Th/target antigenic site conjugates can be improved by delivery through entrapment in or on biodegradable microparticles. In some embodiments, peptide immunogens can be encapsulated with or without an adjuvant, and such microparticles can carry an immune stimulatory adjuvant. In some embodiments, microparticles can be co-administered with peptide immunogens to potentiate immune responses.

### Immunostimulatory Complexes

The present disclosure is also directed to pharmaceutical compositions containing an tau peptide immunogen construct in the form of an immunostimulatory complex with a CpG oligonucleotide. Such immunostimulatory complexes are specifically adapted to act as an adjuvant and as a peptide immunogen stabilizer. The immunostimulatory complexes are in the form of a particulate, which can efficiently present the tau peptide immunogen to the cells of the immune system to produce an immune response. The immunostimulatory complexes may be formulated as a suspension for parenteral administration. The immunostimulatory complexes may also be formulated in the form of w/o emulsions, as a suspension in combination with a mineral salt or with an in-situ gelling polymer for the efficient delivery of the tau peptide immunogen to the cells of the immune system of a host following parenteral administration.

The stabilized immunostimulatory complex can be formed by complexing an tau peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the tau peptide immunogen construct is designed to contain a cationic portion that is positively charged at a pH in the range of 5.0 to 8.0. The net charge on the cationic portion of the tau peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the tau peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the tau peptide immunogen construct is the heterologous spacer. In certain embodiments, the cationic portion of the tau peptide immunogen construct has a charge of +4 when the spacer sequence is (α, ε-N)Lys, ε-N-Lys-Lys-Lys-Lys (SEQ ID NO: 53), or Lys-Lys-Lys-ε-N-Lys (SEQ ID NO: 54).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X1 is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T. In certain embodiments, the CpG oligonucleotide can be CpG1 (SEQ ID NO: 146), CpG2 (SEQ ID NO: 147), or CpG3 (SEQ ID NO: 148).

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

### Applications

The peptide immunogens containing the artificial Th epitopes of the disclosure can be useful in medical and veterinary applications. In some embodiments, the peptide immunogens can be used as vaccines to provide protective immunity from infectious disease, immunotherapies for treating disorders resulting from malfunctioning normal physiological processes, immunotherapies for treating cancer, and as agents to intervene or modify normal physiological processes.

The artificial Th epitopes of the disclosure can provoke an immune response when combined with target B cell epitopes of various microorganisms, proteins, or peptides. In some embodiments, an artificial Th epitopes of the disclosure can be linked to one target antigenic site. In some embodiments, an artificial Th epitope of the disclosure can be linked to two target antigenic sites.

The artificial Th epitopes of the disclosure can be linked to target antigenic sites to prevent and/or treat various diseases and conditions. In some embodiments, a composition of the invention can be used for the prevention and/or treatment of neurodegenerative diseases, infectious diseases, arteriosclerosis, prostate cancer, prevention of boar taint, immunocastration of animals, the treatment of endometriosis, breast cancer and other gynecological cancers affected by the gonadal steroid hormones, and for contraception in males and females. For example, the artificial Th epitopes can be linked to the antigenic sites of the following proteins:
a. Somatostatin to promote growth in farm animals.
b. IgE to treat allergic diseases.
c. The CD4 receptor of Th cells to treat and/or prevent human immunodeficiency virus
(HIV) infections and immune disorders.
d. Foot-and-mouth disease (FMD) virus capsid protein to prevent FMD.
e. HIV virion epitopes to prevent and treat HIV infections.
f. The circumsporozoite antigen of Plasmodium falciparum to prevent and treat
malaria.
g. CETP to prevent and treat arteriosclerosis.
h. Aβ to treat or vaccinate against Alzheimer's disease.
i. Alpha-synuclein to treat or vaccinate against Parkinson's disease.
j. Tau to treat and vaccinate against tauopathies including Alzheimer's disease.
k. IL-31 to treat atopic dermatitis.
l. CGRP for the prevention and treatment of migraine.
m. IAPP (Amylin) for the prevention and treatment of type-2 diabetes.

The use of heterologous artificial Th epitopes has been found to be particularly important for targeting proteins involved in neurodegenerative diseases (e.g., Aβ, alpha-synuclein, Tau). Specifically, peptide immunogens that contain endogenous Th epitopes of targeted neurodegenerative proteins can cause inflammation of the brain when administered to a subject. In contrast, peptide immunogen constructs that contain a heterologous artificial Th epitope liked to an antigenic site of a neurodegenerative protein does not cause brain inflammation.

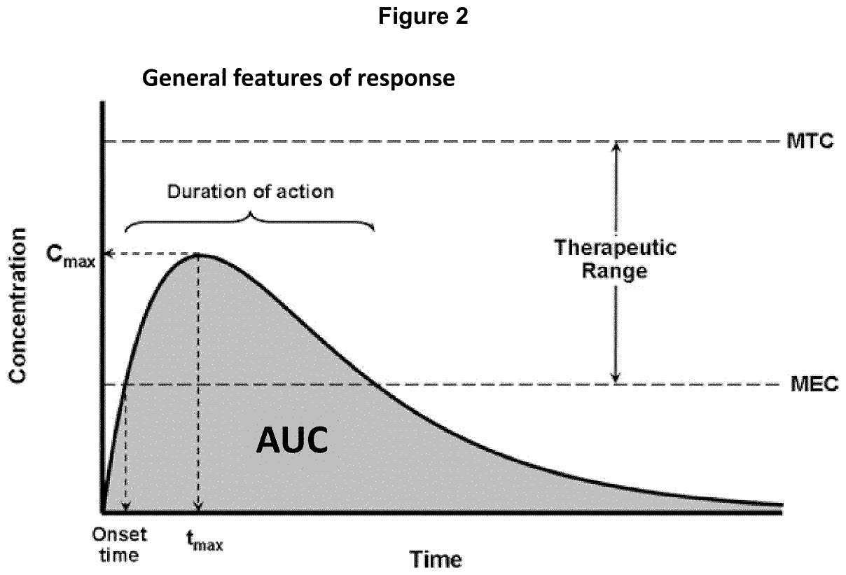

FIG. 2 is a graph illustrating theoretical results that can be obtained using the Th epitope platform described in the present application. The graph shows that the peptide immunogen constructs containing B cell epitopes conjugated to Th epitopes have a fast onset time, reaching Cₘₐₓ quickly. The Cₘₐₓ is within the therapeutic range, which lies between the minimum effective concentration (MEC) and the minimum therapeutic concentration (MTC). The Examples that follow demonstrate that the Cₘₐₓ, duration of action, the onset time, and the tₘₐₓ can all be controlled and/or adjusted by varying the Th epitope that is used, the dosage of the peptide immunogen construct, and the adjuvant.

### Amyloid β

The Aβ peptide is thought to be the pivot for the onset and progression of Alzheimer's disease. Toxic forms of Aβ oligomers and Aβ fibrils are suggested to be responsible for the death of synapses and neurons that lead to the pathology of Alzheimer's disease and dementia. A successful disease-modifying therapy for Alzheimer's disease can include products that affect the disposition of Aβ in the brain.

A peptide immunogen of the disclosure can comprise Th cell epitopes and Aβ-targeting peptides. In some embodiments, the Th cell epitope is Th1 or Th2. In some embodiments, the peptide immunogen can comprise Th1 and Th2. The Aβ-targeting peptide, or B cell epitopes, can be Aβ₁₋₁₄, Aβ₁₋₁₆, Aβ₁₋₂₈, Aβ₁₇₋₄₂, or Aβ₁₋₄₂. In some embodiments, the Aβ-targeting peptide is Ai-14. As used herein, the term Aβ_{x-y} indicates an Aβ sequence from amino acid x to amino acid y of the full-length wild-type Aβ protein.

A peptide immunogen of the disclosure can comprise more than one Aβ-targeting peptide. In some embodiments, a peptide immunogen can comprise two Aβ-targeting peptides. In some embodiments, a peptide immunogen can comprise one Aβ₁₋₁₄ and one Aβ₁₋₄₂ peptide. In some embodiments, a peptide immunogen can comprise two Aβ₁₋₁₄-targeting peptides. In some embodiments, a peptide immunogen can comprise two Aβ₁₋₁₄-targeting peptides, each linked to different Th cell epitopes as a chimeric peptide.

The present disclosure also provides Aβ₁₋₁₄ peptide vaccines comprising two Aβ₁₋₁₄-targeting peptides, each linked to different Th cell epitopes as a chimeric peptide. In some embodiments, a chimeric Aβ₁₋₁₄ peptide can be formulated in a Th1-biased delivery system to minimize T-cell inflammatory reactivity. In some embodiments, a chimeric Aβ₁₋₁₄ peptide can be formulated in a Th2-biased delivery system to minimize T-cell inflammatory reactivity.

### Specific Embodiments

(1) A promiscuous artificial T helper cell (Th) epitope selected from the group consisting of SEQ ID NOs: 32-52.
(2) A peptide immunogen construct represented by the following formulae:

(A)ₙ-(Target antigenic site)-(B)ₒ-(Th)ₘ-(A)ₙ-X

or

(A)ₙ-(Th)ₘ-(B)ₒ-(Target antigenic site)-(A)ₙ-X

or

(A)ₙ-(Th)ₘ-(B)ₒ-(Target antigenic site)-(B)ₒ-(Th)ₘ-(A)ₙ-X

or

{(A)ₙ-(Th)ₚ-(B)ₒ-(Target antigenic site)-(B)ₒ-(Th)ₚ-(A)ₙ-X}m

wherein:
each A is independently an amino acid;
each B is independently a heterologous spacer;
each Th is independently a promiscuous artificial Th epitope of (1);
the Target antigenic site is a B cell epitope from a foreign-antigen protein, a self-antigen protein, or an immunologically reactive analogue thereof;
X is an amino acid, α-COOH, or α-CONH₂;
n is 0, 1, 2, 3, 4, 5, 6, 7, 8, 9, or 10;
m is 1, 2, 3, or 4; and
o is 0, 1, 2, 3, 4, 5, 6, 7, 8, 9, or 10; and
p is 0, 1, 2, 3, or 4.
(3) The peptide immunogen construct of (2), wherein the target antigenic site is a B cell epitope from a foreign-antigen protein selected from the group consisting of a foot-and-mouth disease (FMD) capsid protein, a glycoprotein from porcine reproductive and respiratory syndrome virus (PRRSV), classical swine fever virus (CSFV), human immunodeficiency virus (HIV), and herpes simplex virus (HSV).
(4) The peptide immunogen construct of (2), wherein the target antigenic site is a B cell epitope from a self-antigen protein selected from the group consisting of:
   (a) an Aβ peptide having the amino acid sequence of SEQ ID NO: 56, 57, 58, 59, or 60;
   (b) an alpha-Syn peptide having the amino acid sequence of SEQ ID NO: 61;
   (c) an IgE EMPD peptide having the amino acid sequence of SEQ ID NO: 62;
   (d) a Tau peptide having the amino acid sequence of SEQ ID NO: 63, 69, 70, or 71;
   (e) an IL-31 peptide having the amino acid sequence of SEQ ID NO: 64 or 72; and
   (f) an IL-6 peptide having the amino acid sequence of SEQ ID NO: 145.
(5) The peptide immunogen construct of (2), wherein the heterologous spacer of component B is selected from the group consisting of an amino acid, Lys-, Gly-, Lys-Lys-Lys-, (α, ε-N)Lys, ε-N-Lys-Lys-Lys-Lys (SEQ ID NO: 53), Lys-Lys-Lys-εNLys (SEQ ID NO: 54), Gly-Gly, Pro-Pro-Xaa-Pro-Xaa-Pro (SEQ ID NO: 55), and any combination thereof.
(6) The peptide immunogen construct of (2), wherein the heterologous spacer is selected from the group consisting of (α, ε-N)Lys, ε-N-Lys-Lys-Lys-Lys (SEQ ID NO: 53), and Lys-Lys-Lys-εNLys (SEQ ID NO: 54).
(7) A pharmaceutical composition comprising the peptide immunogen construct according to (2).
(8) A method of preventing and/or treating a disease, condition, or ailment in a subject comprising administering a pharmaceutically affective amount of the pharmaceutical composition of (7) to the subject.
(9) The method according to (8), wherein the a target antigenic site is a B cell epitope from a foreign-antigen protein selected from the group consisting of a foot-and-mouth disease (FMD) capsid protein, a glycoprotein from porcine reproductive and respiratory syndrome virus (PRRSV), classical swine fever virus (CSFV), human immunodeficiency virus (HIV), and herpes simplex virus (HSV).
(10) The method according to (8), wherein the target antigenic site is a B cell epitope from a self-antigen protein selected from the group consisting of:
   (a) an Aβ peptide having the amino acid sequence of SEQ ID NO: 56, 57, 58, 59, or 60;
   (b) an alpha-Syn peptide having the amino acid sequence of SEQ ID NO: 61;
   (c) an IgE EMPD peptide having the amino acid sequence of SEQ ID NO: 62;
   (d) a Tau peptide having the amino acid sequence of SEQ ID NO: 63, 69, 70, or 71;
   (e) an IL-31 peptide having the amino acid sequence of SEQ ID NO: 64 or 72; and
   (f) an IL-6 peptide having the amino acid sequence of SEQ ID NO: 145.
(11) A peptide immunogen construct represented by the following formulae:

(A)ₙ-(Target antigenic site)-(B)ₒ-(Th)ₘ-(A)ₙ-X

or

(A)ₙ-(Th)ₘ-(B)ₒ-(Target antigenic site)-(A)ₙ-X

or

(A)ₙ-(Th)ₘ-(B)ₒ-(Target antigenic site)-(B)ₒ-(Th)ₘ-(A)ₙ-X

or

{(A)ₙ-(Th)ₚ-(B)ₒ-(Target antigenic site)-(B)ₒ-(Th)ₚ-(A)ₙ-X}m

wherein:
each A is independently an amino acid;
each B is independently a heterologous spacer;
each Th is independently a promiscuous artificial Th epitope selected from the group consisting of SEQ ID NOs: 1-52;
the Target antigenic site is a CTL epitope, a Tumor-Associated Carbohydrate Antigen (TACA), a B cell epitope from a neoantigen, a small molecule drug, or an immunologically reactive analogue thereof;
X is an amino acid, α-COOH, or α-CONH₂;
n is 0, 1, 2, 3, 4, 5, 6, 7, 8, 9, or 10;
m is 1, 2, 3, or 4; and
o is 0, 1, 2, 3, 4, 5, 6, 7, 8, 9, or 10; and
p is 0, 1, 2, 3, or 4.
(12) The peptide immunogen of (11), wherein the target antigenic site is a CTL epitope having an amino acid sequence selected from the group consisting of SEQ ID NOs: 76-144.
(13) The peptide immunogen of (12), wherein the target antigenic site is a CTL epitope from HIV selected from the group consisting of SEQ ID NOs: 76-82.
(14) The peptide immunogen of (12), wherein the target antigenic site is a CTL epitope from HSV selected from the group consisting of SEQ ID NOs: 83-106.
(15) The peptide immunogen of (12), wherein the target antigenic site is a CTL epitope from FMDV selected from the group consisting of SEQ ID NOs: 107-123.
(16) The peptide immunogen of (12), wherein the target antigenic site is a CTL epitope from PRRSV selected from the group consisting of SEQ ID NOs: 124-142.
(17) The peptide immunogen of (12), wherein the target antigenic site is a CTL epitope from CSFV selected from the group consisting of SEQ ID NOs: 143-144.
(18) The peptide immunogen of (11), wherein the target antigenic site is a TACA selected from the group consisting of GD3, GD2, Globo-H, GM2, Fucosyl GM1, GM2, PSA, Le^{y}, Le^{x}, SLe^{x}, SLe^{a}, Tn, TF, and STn.
(19) The peptide immunogen of (11), wherein the target antigenic site is a B cell epitope from a neoantigen selected from the group consisting of SEQ ID NOs: 73-75.
(20) The peptide immunogen of (11), wherein the target antigenic site is a small molecule drug.
(21) The peptide immunogen construct of (11), wherein the heterologous spacer of component B is selected from the group consisting of an amino acid, Lys-, Gly-, Lys-Lys-Lys-, (α, ε-N)Lys, ε-N-Lys-Lys-Lys-Lys (SEQ ID NO: 53), Lys-Lys-Lys-εNLys (SEQ ID NO: 54), Gly-Gly, Pro-Pro-Xaa-Pro-Xaa-Pro (SEQ ID NO: 55), and any combination thereof.
(22) The peptide immunogen construct of (11), wherein the heterologous spacer is selected from the group consisting of (α, ε-N)Lys, ε-N-Lys-Lys-Lys-Lys (SEQ ID NO: 53), and Lys-Lys-Lys-εNLys (SEQ ID NO: 54).
(23) A pharmaceutical composition comprising the peptide immunogen construct according to (11).
(24) A method of preventing and/or treating a disease, condition, or ailment in a subject comprising administering a pharmaceutically affective amount of the pharmaceutical composition of (23) to the subject.
(25) The method according to (24), wherein the disease, condition, or ailment is HIV and wherein the a target antigenic site is a CTL epitope from HIV selected from the group consisting of SEQ ID NOs: 76-82.
(26) The method according to (24), wherein the disease, condition, or ailment is HSV and wherein the target antigenic site is a CTL epitope from HSV selected from the group consisting of SEQ ID NOs: 83-106.
(27) The method according to (24), wherein the disease, condition, or ailment is FMDV and wherein the target antigenic site is a CTL epitope from FMDV selected from the group consisting of SEQ ID NOs: 107-123.
(28) The method according to (24), wherein the disease, condition, or ailment is PRRSV and wherein the target antigenic site is a CTL epitope from PRRSV selected from the group consisting of SEQ ID NOs: 124-142.
(29) The method according to (24), wherein the disease, condition, or ailment is CSFV and wherein the target antigenic site is a CTL epitope from CSFV selected from the group consisting of SEQ ID NOs: 143-144.
(30) The method according to (24), wherein the disease, condition, or ailment is CSFV and wherein the target antigenic site is a CTL epitope from CSFV selected from the group consisting of SEQ ID NOs: 143-144.
(31) The method according to (24), wherein the disease, condition, or ailment is cancer and wherein the target antigenic site is a TACA selected from the group consisting of GD3, GD2, Globo-H, GM2, Fucosyl GM1, GM2, PSA, Ley, Lex, SLex, SLea, Tn, TF, and STn.
(32) The method according to (24), wherein the disease, condition, or ailment is cancer and wherein the target antigenic site is a B cell epitope from a neoantigen selected from the group consisting of SEQ ID NOs: 73-75.
(33) A method for tailoring an immune response in a subject comprising:
   (a) preparing more than one peptide immunogen construct according to (11), wherein the Target antigenic site remains constant and the Th epitope is different on each peptide immunogen construct;
   (b) preparing more than one pharmaceutical composition, each of which comprises one of the peptide immunogen constructs prepared in (a) and a pharmaceutically acceptable adjuvant or carrier;
   (c) administering each of the pharmaceutical compositions prepared in (b) to different subjects;
   (d) monitoring the immune response in each of the subjects; and
   (e) selecting the pharmaceutical composition that produces a desired immune response.
(34) The method according to (33), wherein the pharmaceutically acceptable adjuvant or carrier in each of the pharmaceutical compositions is the same.
(35) The method according to (33), wherein the pharmaceutically acceptable adjuvant or carrier in each of the pharmaceutical compositions is different.

### Example 1

### Preparation of Peptides and Peptide Immunogen Constructs

Peptides, including peptide immunogen constructs, were synthesized using automated solid-phase synthesis, purified by preparative HPLC, and characterized by matrix-assisted laser desorption ionization-time of flight (MALDI-TOF) mass spectrometry, amino acid analysis, and reverse-phase HPLC.

The Aβ vaccine (UB-311) comprises two peptide immunogens, each with an N-terminal Aβ₁₋₁₄ peptide, synthetically linked through an amino acid spacer to different Th cell epitope peptides (UBITh® epitopes) derived from two pathogen proteins: hepatitis B surface antigen and measles virus fusion protein. Specifically, the peptide immunogen linked to a measles virus fusion protein was Aβ_{1-14-ε}K-KKK-MvF5 Th (SEQ ID NO: 67) and the peptide immunogen linked to a hepatitis B surface antigen was Aβ_{1-14-ε}K-HBsAg3 Th (SEQ ID NO: 68).

UB-311 was formulated in an alum-containing Th2-biased delivery system and contained the peptides Aβ_{1-14-ε}K-HBsAg3 and Aβ_{1-14-ε}K-KKK-MvF5 Th in an equimolar ratio. The two Aβ immunogens were mixed with polyanionic CpG oligodeoxynucleotide (ODN) to form stable immunostimulatory complexes of micron-sized particulates. An aluminum mineral salt (ADJU-PHOS®) was added to the final formulation, along with sodium chloride for tonicity and 0.25% 2-phenoxyethanol as a preservative.

The sequences of several exemplary target antigenic sites (B cell epitopes and CTL epitopes) are shown in Tables 3A and 3B, respectively. The sequences of several exemplary peptide immunogen constructs containing Aβ₁₋₁₄, rat IL-6₇₂₋₈₂, and IgE-EMPD₁₋₃₉, as target antigenic sites covalently linked to a Th epitope are shown in Table 4. The sequences of peptide immunogen constructs containing α-Synuclein₁₁₁₋₁₃₂ covalently linked to various Th epitopes are shown in Table 5. The sequences of peptide immunogen constructs containing IgE-EMPD_{G1-C39} covalently linked to various Th epitopes are shown in Table 6. The sequences of peptide immunogen constructs containing human IL-6₇₃₋₈₃ covalently linked to various Th epitopes are shown in Table 7. The sequences of peptide immunogen constructs containing di-peptide repeat (DPR) sequences covalently linked to UBITh®1 are shown in Table 9. The sequences of peptide immunogen constructs containing LHRH covalently linked to various Th epitopes are shown in Table 10.

### Example 2

### Ranking of Heterologous T Helper Epitopes Derived from Pathogens and their Inclusion in the Alpha-Synuclein₁₁₁₋₁₃₂, IGE Empd₁₋₃₉, and IL-6₇₃₋₈₃ Peptide Immunogen Constructs Design to Enhance the Immunogenicity of these Selected B Epitope Peptides

a. Peptide Immunogen Synthesis

Three short B cell epitope peptides from alpha-Synuclein (AAs 111-132; SEQ ID NO: 61); IgE EMPD (AAs 1-39; SEQ ID NO: 62); and IL-6 (AAs 73-83; SEQ ID NO: 145), that have been extensively characterized for their functional properties were used as representative target antigenic sites. These three B cell epitopes were made into peptide immunogen constructs according to the formula shown below to assess the ability of representative promiscuous artificial Th epitopes (selected from SEQ ID NOs: 1-52) to render the three individual target antigenic sites immunogenic:

(Th)ₘ-(B)ₒ-(Target antigenic site)-X

wherein:
the Th was selected from the artificial Th epitopes disclosed herein and m was 1;
(B)ₒ was a spacer having the amino acid sequence of SEQ ID NO: 53 or 54;
the Target antigenic site was the B cell epitope of SEQ ID NO: 61, 62, or 145; and
X was an amino acid —CONH₂.

The amino acid sequences of the α-Synuclein, IgE EMPD, and IL-6 peptide immunogen constructs produced are shown in Tables 5, 6, and 7, respectively.

b. Formulations Containing Peptide Immunogen Constructs and Immunizations

A representative immunogenicity study was conducted in guinea pigs to rank the relative effectiveness of the respective heterologous T helper epitopes shown in Table 1. After the various α-Synuclein, IgE EMPD, and IL-6 peptide immunogens were produced, the constructs containing the same Th epitope sequences were mixed together in a 1:1:1 ratio as shown in Table 8. For example, the α-Synuclein, IgE EMPD, and IL-6 constructs containing the UBITH®1 epitope (SEQ ID NOs: 149, 178, and 207) were mixed together to prepare Formulation No. 1 shown in Table 8. The mixture of α-Synuclein, IgE EMPD, and IL-6 peptide immunogen constructs were mixed with the adjuvant MONTANIDE ISA50V2 and then formulated as a stabilized immunostimulatory complex using CpG3 oligonucleotide. Each of the 29 formulations shown in Table 8 contained a total 135 μg of peptides (45 μg per peptide) in a volume of 0.5 mL.

The formulations were administered to guinea pigs (3 per group) at 0, 3, and 6 weeks post initial immunization (wpi) via intramuscular (i.m.) injection. Serum samples were taken at 0, 3, 6, and 8 wpi in order to evaluate antibody titer levels.

c. Immunogenicity Results

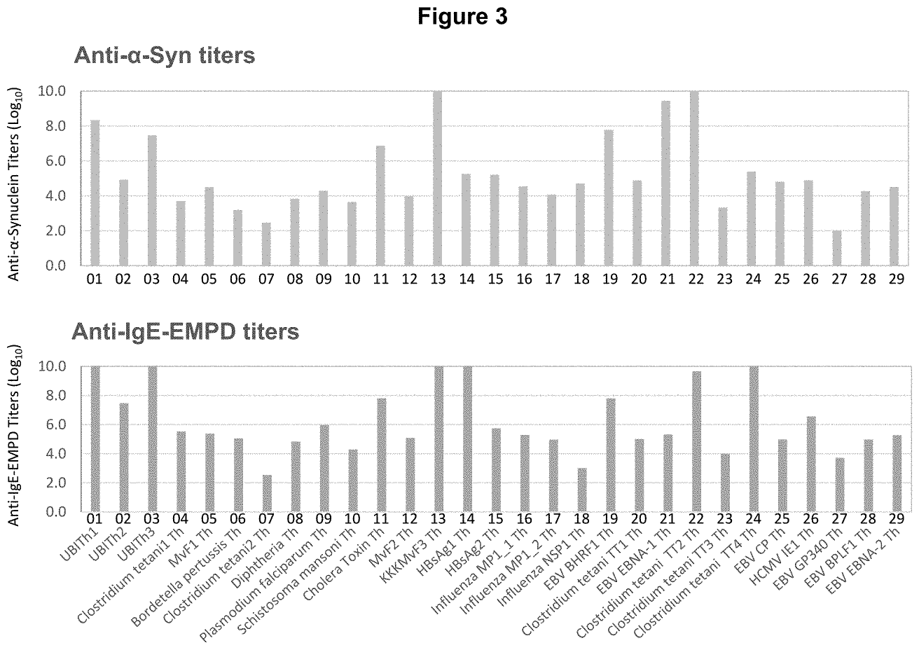

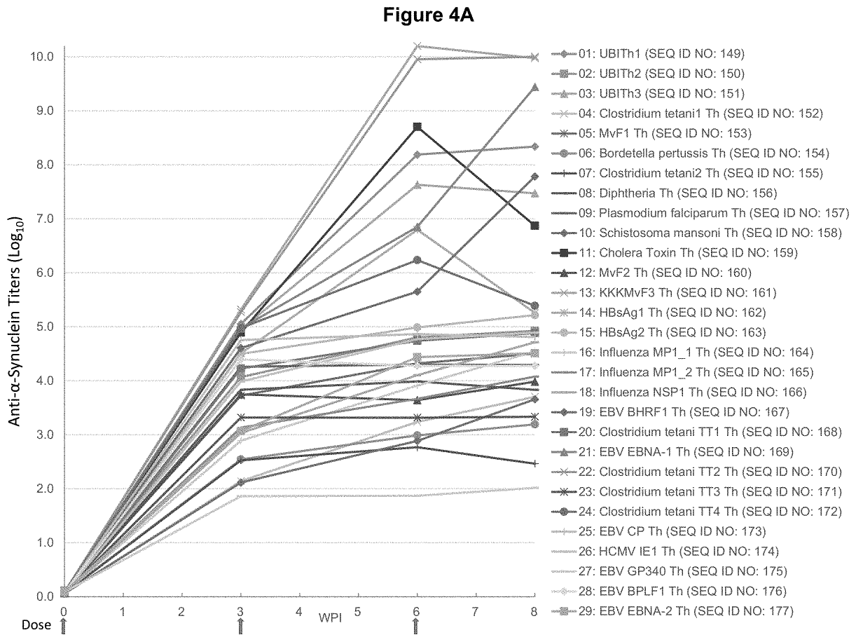

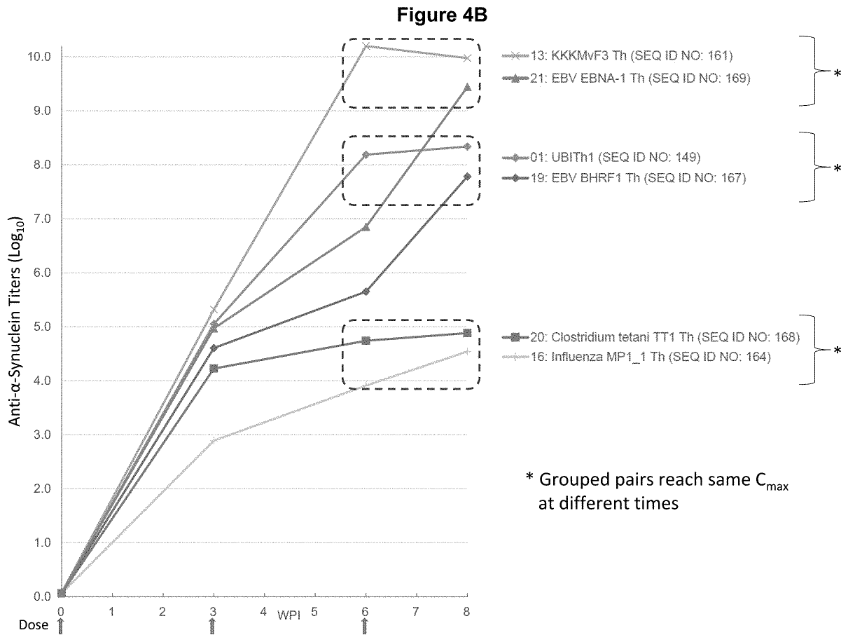

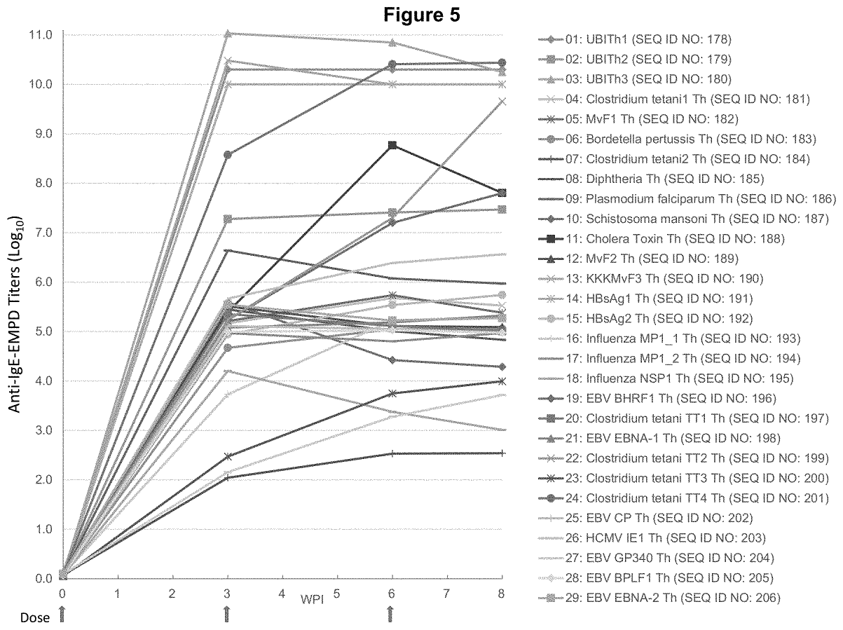

Results obtained at 8 weeks post initial immunization (8 wpi) were used to rank the different α-Synuclein (FIG. 3 upper panel), IgE (FIG. 3 lower Panel) and IL-6 (Table 15) peptide immunogen constructs. Graphs containing the immunogenicity data obtained throughout the study for α-Synuclein are shown in FIG. 4A; IgE-EMPD are shown in FIG. 5; and IL-6 are shown in FIG. 6.

All of the Th epitopes were able to enhance the immunogenicity of the three short B cell epitope peptides to varying degrees. Specifically, the Th epitopes: KKKMvF3 Th (SEQ ID NO: 13), Clostridium tetani TT2 Th (SEQ ID NO: 36), EBV EBNA-1 Th (SEQ ID NO: 42), MvF5 Th; UBITh®1 (SEQ ID NO: 17), EBV BHRF1 Th (SEQ ID NO: 41), MvF4 Th; UBITh®3 (SEQ ID NO: 16), and Cholera Toxin Th (SEQ ID NO: 33) enhanced the immunogenicity of the α-Synuclein peptide (SEQ ID NO: 61) more than the other Th epitopes (FIG. 3 top panel and FIG. 4A). These peptide immunogen constructs are represented by Formulation Nos. 13, 22, 21, 01, 19, 03, and 11, respectively, in Table 5.

For the IgE EMPD peptide (SEQ ID NO: 62), the Th epitopes of Clostridium tetani TT4 Th (SEQ ID NO: 38), UBITh®1 (SEQ ID NO: 17), UBITh®3 (SEQ ID NO: 16), HBsAg1 Th; SSAL2 Th2 (SEQ ID NO: 24), KKKMvF3 Th (SEQ ID NO: 13), Clostridium tetani TT2 Th (SEQ ID NO: 36), Cholera Toxin Th (SEQ ID NO: 33), EBV BHRF1 Th (SEQ ID NO: 41), and HBsAg3 Th; UBITH®2 (SEQ ID NO: 28) enhanced the immunogenicity of the IgE EMPD more than the other Th epitopes (FIG. 3 bottom panel and FIG. 5). These peptide immunogen constructs are represented by Formulation Nos. 24, 01, 03, 14, 13, 22, 11, 19, and 02, respectively, in Table 6.

For the IL-6₇₃₋₈₃ cyclic peptide (SEQ ID NO: 145), the Th epitopes of HBsAg3 Th; UBITH®2 (SEQ ID NO: 28), UBITh®1 (SEQ ID NO: 17), UBITh®3 (SEQ ID NO: 16), Clostridium tetani TT1 Th (SEQ ID NO: 34), and Clostridium tetani TT4 Th (SEQ ID NO: 38) were found to be most potent to enhance the resulting immunogenicity of the IL-6 (Table 15 and FIG. 6). These peptide immunogen constructs are represented by Formulation Nos. 02, 01, 03, 20, and 24, respectively, in Table 7.

These results demonstrate that different immunogenicities towards a single target B cell epitopes can be obtained when using different artificial Th epitopes disclosed herein. Careful calibration of immunogenicity for each peptide immunogen construct in different species, including primates, is required to assure ultimate Th peptide selection and success in the development of a final vaccine formulations.

c. Rate to Cₘₐₓ

A further analysis of the immunogenicity data for the α-Synuclein peptide immunogen constructs covalently linked to different Th epitopes reveals that a particular Cₘₐₓ can be achieved at different rates depending on which Th epitope is utilized. FIG. 4B contains a subset of the immunogenicity data reported in FIG. 4A. Specifically, guinea pigs immunized with Formulation Nos. 13 and 21, which contain the Th epitopes of KKKMvF3 Th (SEQ ID NO: 13) and EBV EBNA-1 Th (SEQ ID NO: 42) covalently linked to the α-Synuclein peptide (SEQ ID NO: 61) achieve the same Cₘₐₓ by 8 wpi, but they do so at different rates. In particular, the α-Synuclein peptide immunogen construct containing KKKMvF3 Th (SEQ ID NO: 13) reaches its Cₘₐₓ faster than the α-Synuclein peptide immunogen construct containing EBV EBNA-1 Th (SEQ ID NO: 42). Similarly, the α-Synuclein peptide immunogen construct containing UBITh®1 (SEQ ID NO: 17) reaches its Cₘₐₓ faster than the α-Synuclein peptide immunogen construct containing EBV BHRF1 Th (SEQ ID NO: 41); and the α-Synuclein peptide immunogen construct containing Clostridium tetani TT1 Th (SEQ ID NO: 34) reaches its Cₘₐₓ faster than the α-Synuclein peptide immunogen construct containing Influenza MP1_1 Th (SEQ ID NO: 48). The results shown in FIG. 4B demonstrate that the choice of Th epitope can affect the rate at which the antibody titers reach their Cₘₐₓ.

d. Summary

The results from this experiment demonstrate that the immune response elicited by the peptide immunogen constructs (including antibody titers, Cₘₐₓ, onset of antibody production, duration of response, etc.) can be modulated by the choice of the Th epitope that is chemically linked to the B cell epitope. Therefore, specific immune responses to target antigenic sites can be designed by varying the Th epitope that is conjugated to the B cell epitope in the peptide immunogen construct, which can facilitate the tailoring of personalized medical treatment to the individual characteristics of any patient or subject.

### Example 3

### Immunogenicity of Peptide Immunogen Constructs can Vary Depending on the Length of the B Cell Epitope Sequence

The immunization and evaluation of three di-peptide repeat (DPR) peptide immunogen constructs are described in detail below.

a. Immunizations and Sera Collection

Three DPR peptide immunogen constructs were produced having the amino acid sequences of SEQ ID NOs: 236, 237, and 238 as shown in Table 9. Each peptide immunogen was formulated in MONTANIDE™ ISA51 and CpG to immunize guinea pigs at dose at 400 μg/ml as prime immunization and 100 μg/ml as boost dose at 3, 6, 9, and 12 weeks post-injection (WPI), 3 guinea pigs per group.

b. Evaluation of Antibody Titers

ELISA assay was performed to evaluate the immunogenicity of the designer DPR peptide immunogen constructs. DPR B cell epitope peptides or peptide immunogen constructs were used to coat the plate wells, which served as targeting peptides. Guinea pig immune serum was diluted from 1:100 to 1:100,000 by 10-fold serial dilutions. The titer of a tested serum, expressed as Log 10, was calculated by linear regression analysis of the A450 nm with the cut off A₄₅₀ set at 0.5. All peptide immunogens induced strong immunogenicity titers against the B epitope peptides coated in the plate wells.

c. Peptide-Based ELISA Tests for Antibody Specificity Analysis

ELISA assays for evaluating immune serum samples were developed as described below.

The wells of 96-well plates were coated individually for 1 hour at 37° C. with 100 μL of same DPR peptide immunogen construct used to immunize the animal (i.e., SEQ ID NOs: 236, 237, or 238), at 2 μg/mL in 10 mM NaHCO₃ buffer, pH 9.5.

d. Assessment of Antibody Reactivity Towards DPRs by ELISA Tests

The peptide-coated wells were incubated with 250 μL of 3% by weight of gelatin in PBS in 37° C. for 1 hour to block non-specific protein binding sites, followed by three washes with PBS containing 0.05% by volume of TWEEN® 20 and dried. Sera to be analyzed were diluted 1:20 (unless noted otherwise) with PBS containing 20% by volume normal goat serum, 1% by weight gelatin and 0.05% by volume TWEEN® 20. One hundred microliters (100 μL) of the diluted specimens (e.g., serum, plasma) were added to each of the wells and allowed to react for 60 minutes at 37° C. The wells were then washed six times with 0.05% by volume TWEEN® 20 in PBS in order to remove unbound antibodies. Horseradish peroxidase (HRP)-conjugated species (e.g., mouse, guinea pig, or human) specific goat anti-IgG, was used as a labeled tracer to bind with the antibody/peptide antigen complex formed in positive wells. One hundred microliters of the peroxidase-labeled goat anti-IgG, at a pre-titered optimal dilution and in 1% by volume normal goat serum with 0.05% by volume TWEEN® 20 in PBS, was added to each well and incubated at 37° C. for another 30 minutes. The wells were washed six times with 0.05% by volume TWEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0M H₂SO₄ and absorbance at 450 nm (A₄₅₀) determined. For the determination of antibody titers of the immunized animals that received the various DPR derived peptide immunogens, 10-fold serial dilutions of sera from 1:100 to 1:10,000 were tested, and the titer of a tested serum, expressed as Log₁₀, was calculated by linear regression analysis of the A₄₅₀ with the cutoff A₄₅₀ set at 0.5.

e. Immunogenicity Evaluation

Preimmune and immune serum samples from animals were collected according to experimental immunization protocols and heated at 56° C. for 30 minutes to inactivate serum complement factors. Following the administration of the pharmaceutical composition containing a DPR peptide immunogen construct, blood samples were obtained according to protocols and their immunogenicity against specific target site(s) evaluated. Serially diluted sera were tested and positive titers were expressed as Log₁₀ of the reciprocal dilution. Immunogenicity of a particular pharmaceutical composition is assessed by its ability to elicit high titer B cell antibody response directed against the desired epitope specificity within the target antigen while maintaining a low to negligible antibody reactivity towards the Th epitope employed to provide enhancement of the desired B cell responses.

f. Immunoassay for DPR Level in Mouse Immune Sera

Serum DPR levels in mice receiving DPR derived peptide immunogens were measured by a sandwich ELISA (Cloud-clon, SEB222Mu) using anti-DPR antibodies as capture antibody and biotin-labeled anti-DPR antibody as detection antibody. Briefly, the antibody was immobilized on 96-well plates at 100 ng/well in coating buffer (15 mM Na₂CO₃, 35 mM NaHCO₃, pH 9.6) and incubated at 4° C. overnight. Coated wells were blocked with 200 μL/well of assay diluents (0.5% BSA, 0.05% TWEEN®-20, 0.02% ProClin 300 in PBS) at room temperature for 1 hour. Plates were washed 3 times with 200 μL/well of wash buffer (PBS with 0.05% TWEEN®-20). Purified recombinant DPR was used to generate a standard curve (range 156 to 1,250 ng/mL by 2-fold serial dilution) in assay diluent with 5% mouse sera. Fifty microliters (50 μL) of the diluted sera (1:20) and standards were added to coated wells. The incubation was carried out at room temperature for 1 hour. All wells were aspirated and washed 6 times with 200 μL/well of wash buffer. The captured DPR was incubated with 100 μL of detection antibody solution (50 ng/ml of biotin labeled HP6029 in assay diluent) at room temperature for 1 hour. Then, the bound biotin-HP6029 was detected using streptavidin poly-HRP (1:10,000 dilution, Thermo Pierce) for 1 hour (100 μL/well). All wells were aspirated and washed 6 times with 200 μL/well of wash buffer and the reaction was stopped by addition of 100 μL/well of 1M H₂SO₄. The standard curve was created by using the SoftMax Pro software (Molecular Devices) to generate a four parameter logistic curve-fit and used to calculate the concentrations of DPR in all tested samples. Student t tests were used to compare data by using the Prism software.

g. Purification of Anti-DPR Antibodies

Anti-DPR antibodies were purified from sera collected at 3 to 15 weeks post-injection (WPI) of guinea pigs or mice immunized with DPR peptide immunogen constructs containing peptides of different sequences (SEQ ID NOs: 236, 237, or 238) by using an affinity column (Thermo Scientific, Rockford). Briefly, after buffer (0.1 M phosphate and 0.15 M sodium chloride, pH 7.2) equilibration, 400 μL of serum was added into the Nab Protein G Spin column followed by end-over-end mixing for 10 min and centrifugation at 5,800×g for 1 min. The column was washed with binding buffer (400 μL) for three times. Subsequently, elution buffer (400 μL, 0.1 M glycine pH 2.0) was added into the spin column to elute the antibodies after centrifuging at 5,800×g for 1 min. The eluted antibodies were mixed with neutralization buffer (400 μL, 0.1 M Tris pH 8.0) and the concentrations of these purified antibodies were measured by using Nan-Drop at OD280, with BSA (bovine serum albumin) as the standard.

h. Results

The immunogenicity titer against DPR peptides or peptide immunogens from the immunized guinea pig serum was assessed by ELISA.

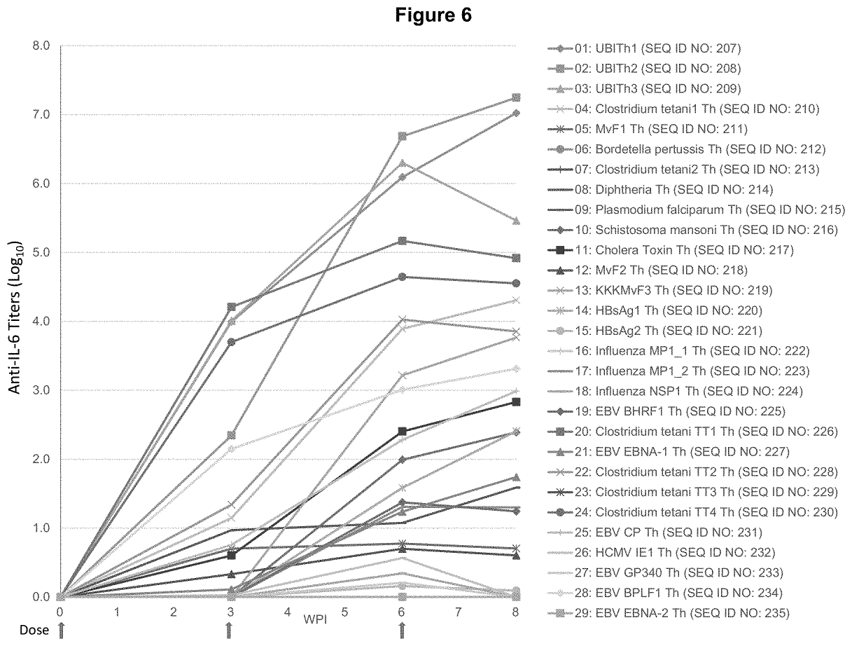

FIG. 7 shows the properties of antisera over a 15-week period in guinea pigs immunized with 3 different DPR peptide immunogen constructs. The guinea pig antisera from 0, 3, 6, 9, 12 and 15 wpi were diluted by a 10-fold serial dilution. ELISA plates were coated with DPR peptides or peptide immunogens. The titer of a tested serum, expressed as Log₁₀, was calculated by linear regression analysis of the A450 nm with the cutoff A₄₅₀ set at 0.5.

The ELISA data for DPR peptide immunogen constructs containing poly-GA peptides (SEQ ID NOs: 236, 237, and 238) were then plotted as graphs shown in FIG. 7, where the same peptide immunogen that was used to immunize the animal was bound to the ELISA plate for analysis. All of the DPR immunogen constructs, demonstrated high immunogenicity to the corresponding DPR peptides or peptide immunogens. The ELISA results showed no detectable antibody titer was observed in each group prior to immunization at week 0. The data show that the length of dipeptide repeats in the DPR peptide immunogen constructs can have a moderate effect on antibody titers. Specifically, poly-GA 10, 15 and 25 repeats constructs (SEQ ID NO: 236, 237, and 238, respectively) have different immunogenicities compared to each other, as shown in FIG. 7.

Interestingly, the lengths of the B cell epitope peptide can have an effect on the immunogenicity profile of the peptide immunogen construct. FIG. 7 (left graph) shows that the immunogenicity of the GA₁₀ peptide immunogen construct (SEQ ID NO: 236) steadily increases over time with regular boosts, while FIG. 7 (middle graph) shows that the immunogenicity of the GA₁₅ peptide immunogen construct (SEQ ID NO: 237) peaks at around 9 wpi before decreasing, and FIG. 7 (right graph) shows that the immunogenicity of the GA₂₅ peptide immunogen construct (SEQ ID NO: 238) steadily increases over time with regular boosts.

The results from FIG. 7 demonstrate that the immune response can be affected depending on the length of the B cell epitope that is used in the peptide immunogen construct.

i. Summary

The results from this experiment demonstrate that the immune response elicited by the peptide immunogen constructs (including antibody titers, Cₘₐₓ, onset of antibody production, duration of response, etc.) can be modulated by the length of the B cell epitope used in the peptide immunogen construct. Therefore, specific immune responses to target antigenic sites can be designed by varying the length of the B cell epitope in the peptide immunogen construct, which can facilitate the tailoring of personalized medical treatment to the individual characteristics of any patient or subject.

### Example 4

### Immunogenicity of Peptide Immunogen Constructs can Vary Depending on the Amount of Peptide Administered and the Dosing Regimen

The immunization and evaluation of various doses and the dosing regimen of peptide immunogen constructs are described in detail below.

a. UB-311 Vaccine (Aβ Peptide Immunogens)

The Aβ vaccine (UB-311) comprises two peptide immunogens, each with an N-terminal Aβ₁₋₁₄ peptide, synthetically linked through an amino acid spacer to different Th cell epitope peptides (UBITh® epitopes) derived from two pathogen proteins: hepatitis B surface antigen and measles virus fusion protein. Specifically, the peptide immunogen linked to a measles virus fusion protein was Aβ_{1-14-ε}K-KKK-MvF5 Th (SEQ ID NO: 67) and the peptide immunogen linked to a hepatitis B surface antigen was Aβ_{1-14-ε}K-HBsAg3 Th (SEQ ID NO: 68).

UB-311 was formulated in an alum-containing Th2-biased delivery system and contained the peptides Aβ₁₋₁₄-εK-HBsAg3 and Aβ₁₋₁₄-εK-KKK-MvF5 Th in an equimolar ratio. The two Aβ immunogens were mixed with polyanionic CpG oligodeoxynucleotide (ODN) to form stable immunostimulatory complexes of micron-sized particulates. An aluminum mineral salt (ADJU-PHOS®) was added to the final formulation, along with sodium chloride for tonicity and 0.25% 2-phenoxyethanol as a preservative.

b. Different Doses of UB-311 in Guinea Pigs

The Aβ vaccine (UB-311) was administered to guinea pigs at 0, 3, and 6 wpi at doses containing 0 μg, 1 μg, 3 μg, 10 μg, 30 μg, 100 μg, 300 μg, 600 μg, and 1,000 μg of total peptide immunogen constructs. Serum samples were taken at 0, 3, 5, 7, and 9 wpi to evaluate the antibody titers.

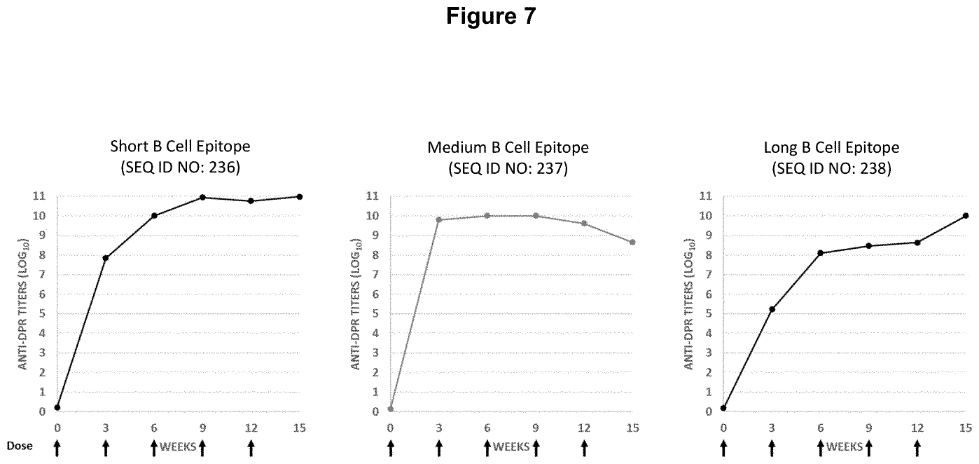

FIG. 8 is a graph that shows the antibody titer results obtained for each immunization dose. The results demonstrate that the amount of peptide used in the immunization can have an appreciable effect on the antibody titers; however, the optimal technical effect should be evaluated for each dose. Specifically, FIG. 8 shows that increasing the dose of peptide administered from 0 μg to 600 μg directly corresponds to an increase in immunogenicity. However, when 1,000 μg of peptide immunogen is administered, the immunogenicity can actually be reduced compared to lower doses of peptide immunogen constructs (compared 300 μg and 600 μg with 1,000 μg). Therefore, the optimal immunogenicity obtained by a peptide immunogen construct is not linear and should be carefully evaluated for each peptide immunogen construct.

c. Different Dosing Regimens of UB-311 in Guinea Pigs can Effect Immunogenicity

The dosing regimen of the Aβ vaccine (UB-311) was evaluated to determine if the amount of peptide immunogen construct administered as a prime dose and a booster dose can affect the overall immunogenicity of the composition.

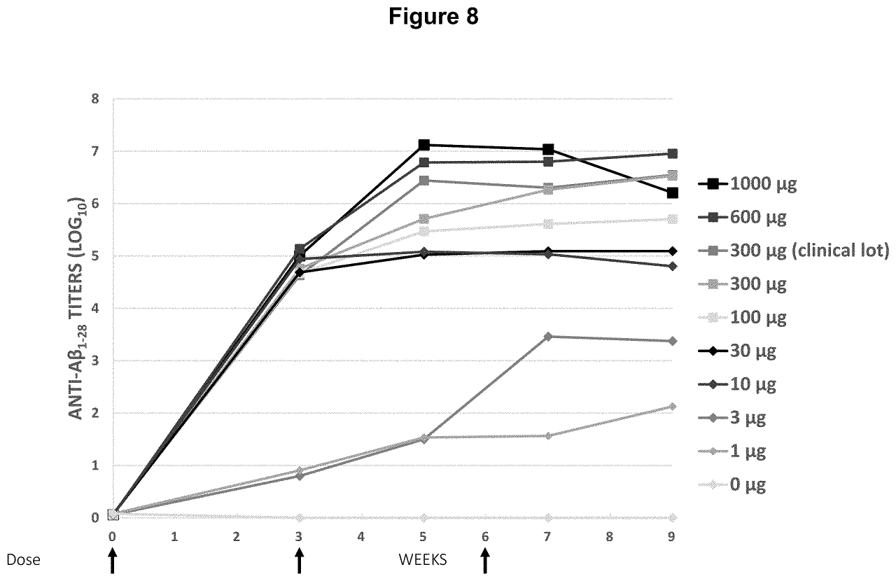

The UB-311 vaccine was administered to guinea pigs at 0, 3, and 6 wpi with different prime and booster doses administered. Specifically, one group of animals were primed with a dose of 100 μg of UB-311 at week 0 wpi and boosted with 2 doses of 400 μg of UB-311; whereas a second group of animals were primed with a dose of 400 μg of UB-311 at week 0 wpi and boosted with 2 doses of 100 μg of UB-311. The results from this experiment are shown in FIG. 9.

FIG. 9 shows that the dosing regimen can have an effect on the immunogenicity (Cₘₐₓ and duration) of the UB-311 composition. Specifically, animals that were primed with a low dose (100 μg of UB-311) and boosted with a high dose (400 μg of UB-311) achieved a higher and longer Cₘₐₓ compared to animals that were primed with a high dose (400 μg of UB-311) and boosted with a lower dose (100 μg of UB-311).

The results from this study demonstrate that the dosing regimen can affect the immunogenicity of the peptide immunogen constructs.

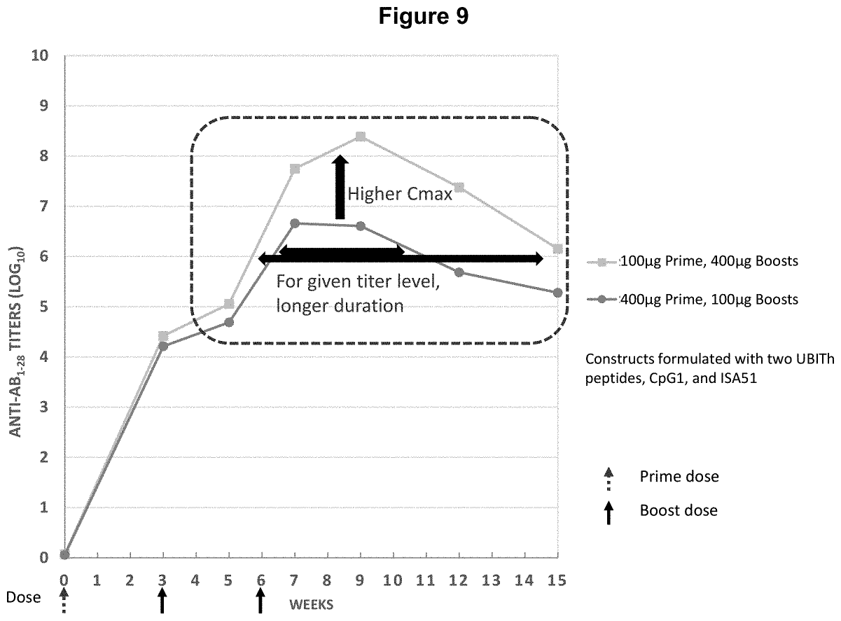

FIG. 10 provides an additional example that the dosing regimen can affect the immunogenicity of the peptide immunogen constructs. Specifically, FIG. 10 (top panel) shows the anti-Aβ₁₋₂₈ antibody titers by ELISA obtained after immunizing human subjects with 300 μg of the Aβ vaccine (UB-311) over a 3 month boosting regimen (top panel) or a 6 month boosting regimen (bottom panel) with the Aβ vaccine (UB-311). The boxed portion in each graph highlights the average titers for all human subjects in the study.

The results from FIG. 10 demonstrate that a different immunogenicity profile can be achieved depending on the dosing regimen provided to the subjects.

d. Different Dosing Regimens of LHRH Peptide Immunogen Constructs in Rats

The dosing regimen of formulations containing different amounts of LHRH peptide immunogen constructs was evaluated to determine if the total amount of peptide immunogen construct can affect the immunogenicity of the formulations.

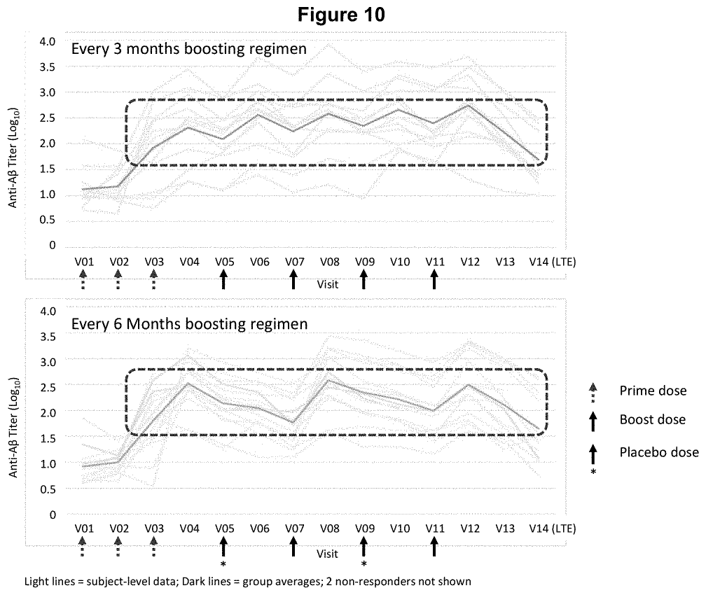

Specifically, three rats were immunized with an LHRH composition containing the 3 peptides shown in Table 10. One group of rats were immunized with 100 μg of the 3 LHRH peptide immunogens; whereas a second group of rats were immunized with 300 μg of the 3 LHRH peptide immunogens. The immunogenicity and testosterone concentrations were evaluated and are reported in FIGS. 11A and 11B.

FIG. 11A shows the antibody titers and testosterone concentrations obtained after immunizing rats with 100 μg amount of the LHRH formulation. FIG. 11B shows the antibody titers and testosterone concentrations obtained after immunizing rats with 300 μg amount of the LHRH formulation. FIGS. 11A and 11B demonstrate that, for the LHRH peptide immunogen constructs, the higher dose of 300 μg results in higher anti-LHRH titers and higher reduction in testosterone concentrations compared to the lower dose of LHRH peptide immunogen constructs.

Therefore, the results in FIGS. 11A and 11B demonstrate that there is a direct correlation between the dosage levels of the peptide immunogen constructs and the technical effect achieved.

e. Summary

The results from this experiment demonstrate that the immune response elicited by the peptide immunogen constructs (including antibody titers, Cₘₐₓ, onset of antibody production, duration of response, etc.) can be modulated by using different dosing regimens. Therefore, specific immune responses to target antigenic sites can be designed by varying the dosing regimen in a patient or subject, which can facilitate the tailoring of personalized medical treatment to the individual characteristics of any patient or subject.

### Example 5

### Immunogenicity of Peptide Immunogen Constructs can Vary Depending on the Adjuvant Used

The immunization and evaluation of IL-6, IgE EMPD, and LHRH peptide immunogen constructs formulated in different adjuvants were evaluated, as described below.

a. IL-6 Peptide Immunogen Constructs

The immunogenicity of formulations containing an IL-6 peptide immunogen construct utilizing different adjuvants was evaluated. The POC study in CIA rats demonstrated that the designed peptide immunogen constructs with high immunogenicity and therapeutic efficacy against IL-6 induced pathogenesis that implicates a potential immunotherapeutic application in rheumatoid arthritis and other autoimmune diseases. The following studies focused on the optimization of the peptide immunogen constructs and selection of adjuvants as well as the dose determination in CIA Lewis rats.

MONTANIDE ISA 51 and ADJU-PHOS as different adjuvants formulated with same peptide immunogen (SEQ ID NO: 243) plus CpG respectively were evaluated in a rat CIA immunization study. Five rats assigned into each of 5 groups received one of two adjuvant formulations, total 10 groups for these two different adjuvants. All animals in the treatment groups were injected by different doses at 5, 15, 45, 150 μg in 0.5 ml through i.m. route in prime and boosts at day −7, 7, 14, 21 and 28 with clinical observation till to day 35. Two different adjuvant placebo groups without peptide immunogen received injection with only adjuvant vehicles in the formulation.

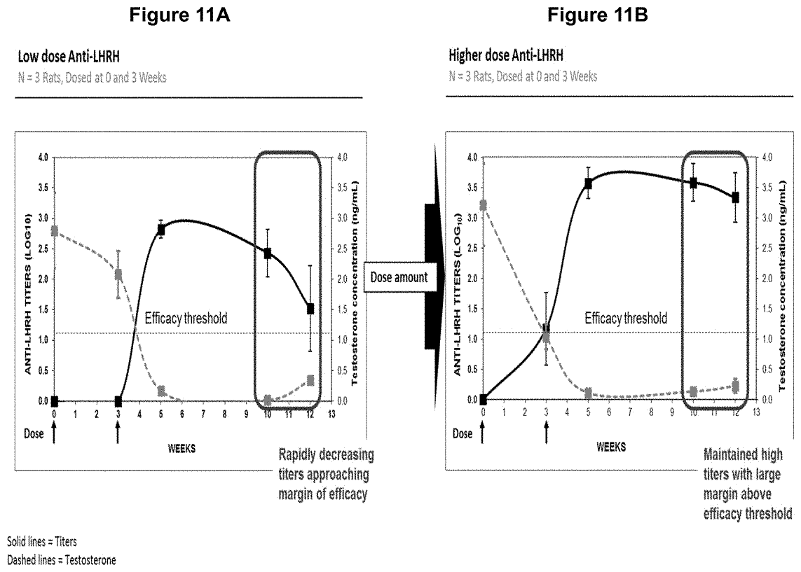

Anti-IL-6 titer was measured by ELISA against rat IL-6 recombinant protein coated in the plate wells. Results showed none of the two placebo groups injected with two different adjuvant vehicles was found detectable anti-IL-6 antibody titers, while all treatment groups immunized with IL-6 immunogen construct (SEQ ID NO: 243) with both adjuvant formulations generated antibody against rat IL-6 by ELISA. Generally speaking, the result showed that a dose dependent manner was observed, especially for the groups with ISA 51 formulation (FIG. 12). The ISA 51 formulation induced higher immune response than ADJUPHOS formulation in immunized rats, with immunogenicities of the ISA 51 formulations having Log₁₀ values over 4 from all doses, respectively, compared to immunogenicities below 4 (log₁₀) for formulations prepared with the ADJUPHOS adjuvant.

FIG. 12 illustrates the kinetics of antibody response over a 43-day period in rats immunized with different doses of SEQ ID NO: 243 formulated with either ISA 51/CpG or ADJU-PHOS/CpG. ELISA plates were coated with recombinant rat IL-6. Serum was diluted from 1:100 to 1:4.19×10⁸ by a 4-fold serial dilution. The titer of a tested serum, expressed as Log₁₀, was calculated by incorporating a cutoff of 0.45 into a four-parameter logistic curve of each serum sample with nonlinear regression.

The results from this experiment demonstrate that the choice of adjuvant can have a significant technical effect of the immunogenicity of the peptide immunogen construct.

### b. 12E EMPD Peptide Immunogen Constructs

The immunogenicity of formulations containing an IgE EMPD peptide immunogen construct utilizing different adjuvants was evaluated. The POC study in macaques demonstrated that the designed peptide immunogen constructs with high immunogenicity and therapeutic efficacy against IgE EMPD induced pathogenesis that implicates a potential immunotherapeutic application. The following studies focused on the optimization of the peptide immunogen constructs and selection of adjuvants using the IgE EMPD peptide immunogen constructs.

ADJU-PHOS and MONTANIDE ISA 51 as different adjuvants formulated with same IgE EMPD peptide immunogen (SEQ ID NO: 178) plus CpG were evaluated in a macaque immunization study.

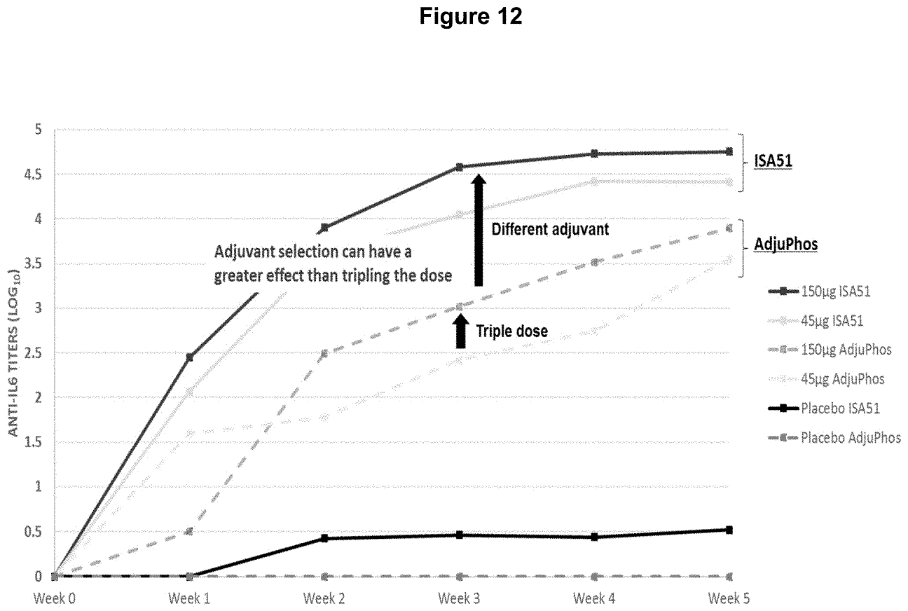

FIGS. 13A and 13B show graphs illustrating the anti-IgE-EMPD antibody titers obtained after immunizing macaques with various amounts of the IgE-EMPD peptide immunogen construct of SEQ ID NO: 178 in different adjuvants. FIG. 13A shows the antibody titers obtained using ADJUPHOS as an adjuvant formulated as a stabilized immunostimulatory complex using CpG3; whereas FIG. 13B shows the antibody titers obtained using MONTANIDE ISA51 as an adjuvant formulated as a stabilized immunostimulatory complex using CpG3.

The results from this experiments demonstrate that the IgE EMPD peptide immunogen constructs formulated in different adjuvants are more immunogenic when used with MONTANTIDE ISA51 compared to formulations containing ADJUPHOS (FIGS. 13A and 13B). Therefore, the use of different adjuvants can produce a different technical effect (immunogenicity) of the peptide immunogen constructs.

c. LHRH Peptide Immunogen Constructs

The immunogenicity of formulations containing LHRH peptide immunogen constructs utilizing different adjuvants was evaluated. The POC study in pigs demonstrated that the designed peptide immunogen constructs with high immunogenicity and therapeutic efficacy against LHRH induced pathogenesis that implicates a potential immunotherapeutic application. The following studies focused on the optimization of the peptide immunogen constructs and selection of adjuvants using the LHRH peptide immunogen constructs.

Emulsigen D and MONTANIDE ISA50V as different adjuvants formulated with same LHRH peptide immunogens (SEQ ID NOs: 239-241) at the same concentration were evaluated in a pig immunization study.

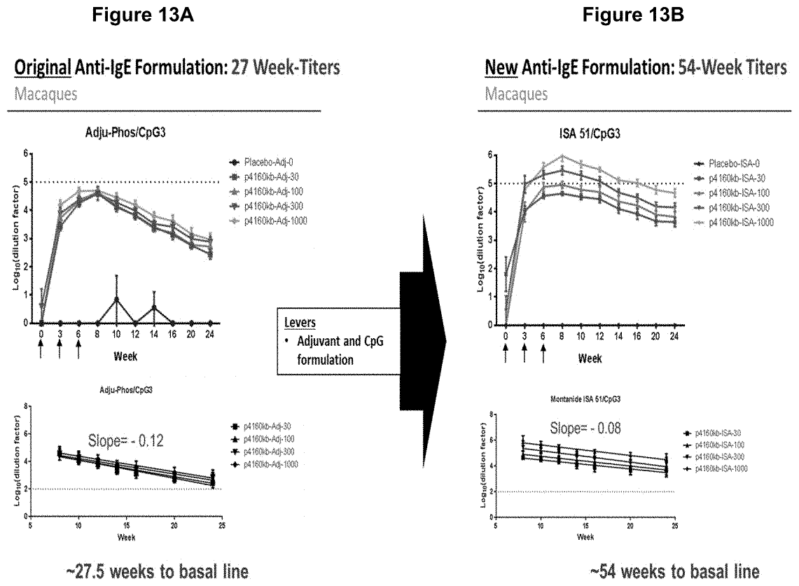

FIGS. 14A and 14B show graphs illustrating the anti-LHRH antibody titers obtained after immunizing pigs with various amounts of the LHRH peptide immunogen constructs of SEQ ID NOs: 239-241 in different adjuvants. FIG. 14A shows the antibody titers obtained using Emulsigen D as an adjuvant; whereas FIG. 14B shows the antibody titers obtained using MONTANIDE ISA50V as an adjuvant.

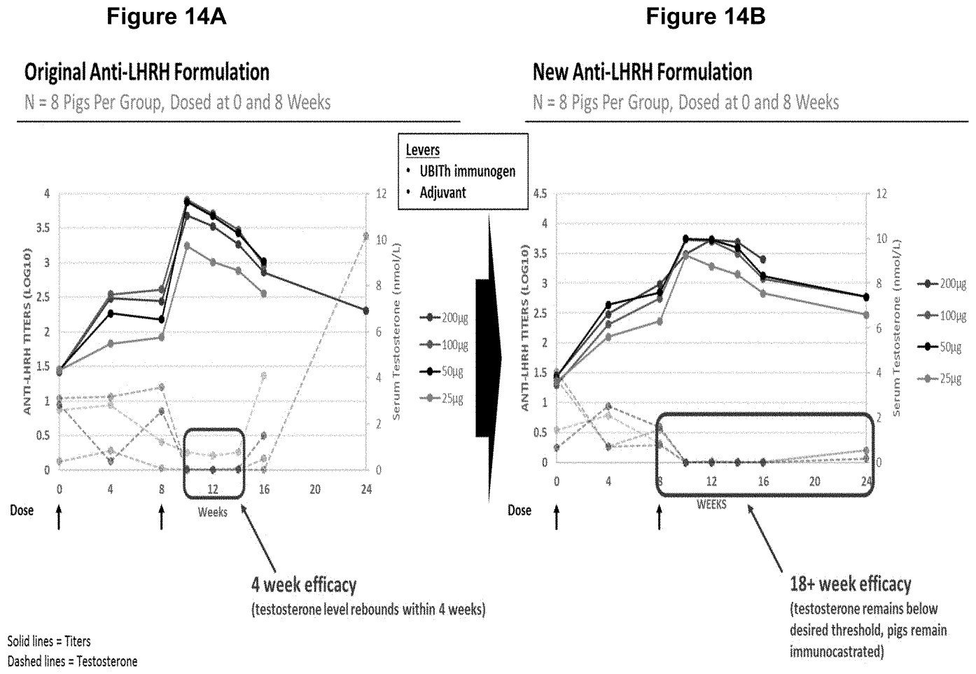

The results from this experiments demonstrate that the LHRH peptide immunogen constructs formulated in different adjuvants have a different technical effect (reduction in testosterone concentration) when formulated with MONTANTIDE ISA50V compared to formulations containing Emulsigen D (FIGS. 15A and 14B). Therefore, the use of different adjuvants can produce a different technical effect (duration of effect, i.e. immunocastration in this case) of the peptide immunogen constructs.

d. Summary

The results from this experiment demonstrate that the immune response elicited by the peptide immunogen constructs (including antibody titers, Cₘₐₓ, onset of antibody production, duration of response, etc.) can be modulated by the choice of adjuvant used in the formulation containing the peptide immunogen constructs at the same concentration. Therefore, specific immune responses to target antigenic sites can be designed by varying either the Th epitope that is chemically linked to the B cell epitope or the adjuvant used in the formulation, which can facilitate the tailoring of personalized medical treatment to the individual characteristics of any patient or subject.

### Example 6

### Exclusive Immunogenicity of Peptide Immunogen Constructs in Guinea Pigs that Target Abeta Peptides but not Th Epitopes

Six guinea pigs were immunized at weeks 0 and 4 with peptide immunogen constructs Aβ_{1-14-ε}K-KKK-MvF5 (SEQ ID NO: 67) and Aβ_{1-14-ε}K-HBsAg3 (SEQ ID NO: 68) formulated together in equimolar ratio. At week 8, animals were bled and serum samples were collected to determine anti-Aβ peptide and anti-Th epitope antibody titers (log₁₀) by ELISA test. The antibody response of all 6 guinea pigs specifically targeted the Aβ₁₋₄₂ peptide and not the two artificial Th epitopes (MvF5 Th and HBsAg3 Th), as shown in Table 11.

### Example 7

### Cellular Immune Response in Baboons and Macaque Peripheral Blood Mononuclear Cell (PBMC) Cultures from Animals Immunized with the UB-311 Vaccine

Peripheral blood mononuclear cells (PBMC) from baboons and from Cynomolgus macaques immunized with UB-311 were isolated by Ficoll-hypaque gradient centrifugation. For peptide-induced proliferation and cytokine production, cells (2×10⁵ per well) were cultured alone or with individual peptide domains added (including, Aβ₁₋₁₄, Aβ₁₋₄₂, UBITh®, and non-relevant peptide). Mitogens (PHA, PWM, Con A) were used as positive controls (10 μg/mL at 1% v/v of culture). On day 6, 1 μCi of 3H-thymidine (3H-TdR) was added to each of three replicate culture wells. After 18 h of incubation, cells were harvested and 3H-TdR incorporation was determined. The stimulation index (S.I.) represents the cpm in the presence of antigen divided by the cpm in the absence of antigen; a S.I.>3.0 was considered significant.

Cytokine analyses (IL-2, IL-6, IL-10, IL-13, TNFα, IFNγ) from the Cynomolgus macaque PMBC cultures were performed on aliquots of culture medium alone or in the presence of peptide domains or mitogens. Monkey-specific cytokine sandwich ELISA kits (U-CyTech Biosciences, Utrecht, The Netherlands) were used to determine the concentration of individual cytokines following kit instructions.

PMBCs were isolated from whole blood collected from macaques at 15, 21, and 25.5 weeks of the immunized animals. The isolated PBMCs were cultured in the presence of various Aβ peptides (Aβ₁₋₁₄ and Aβ₁₋₄₂).

No proliferation responses by lymphocytes were observed when Aβ₁₋₁₄ peptide was added to the culture medium. However, positive proliferation responses were found when the Aβ₁₋₄₂ peptide was added to the PBMC cultures.

The PBMC samples collected at 15, 21 and 25.5 weeks were also tested for cytokine secretion in the presence of Aβ peptides or PHA mitogen. As shown in Table 12, three cytokines (IL-2, IL-6, TNFα) showed detectable secretion in response to the full-length Aβ₁₋₄₂ peptide but not to the Aβ₁₋₁₄ peptide; up-regulation of cytokine secretion was not detected in the UBITh® AD vaccine-treated samples when compared to the placebo vaccine samples. Three other cytokines (IL-10, IL-13, IFNγ) tested in the presence of the Aβ peptides were below the assay detection limit in all PBMC cultures.

The macaques were immunized with the UB-311 vaccine having only the N-terminal Aβ₁₋₁₄ peptide immunogens with foreign T helper epitopes, without the Aβ₁₇₋₄₂ peptide domain, indicating that the positive proliferation results noted in the PBMC cultures in the presence of Aβ₁₋₄₂ peptide were not related to the UB-311 vaccine response, but rather were a background response to native full length Aβ.

These results support the safety of the UB-311 vaccine that has only Aβ₁₋₁₄ and foreign T helper epitopes, showing that it does not generate potentially inflammatory anti-self cell-mediated immune responses to the native full length Aβ peptides in the normal macaques. In contrast, the adverse events associated with encephalitis in the clinical trial studies of the AN-1792 vaccine were attributed in part, to the inclusion of T cell epitopes within the monomeric or fibrillar/aggregated Aβ₁₋₄₂ immunogen of that vaccine.

### Example 8

### Lymphocyte Proliferation Analysis and Cytokine Analysis of PBMC from Alzheimer's Patients Immunized with UB311 Vaccine

Peripheral blood mononuclear cells (PBMC) from patients with Alzheimer's Disease were isolated by Ficoll-hypaque gradient centrifugation. For peptide-induced proliferation and cytokine production, cells (2.5×10⁵ per well) were cultured in triplicate alone or with individual peptide domains added (at a final concentration of 10 μg/mL), including Aβ₁₋₁₄ (SEQ ID NO: 56), Aβ₁₋₁₆ (SEQ ID NO: 57), Aβ₁₋₂₈ (SEQ ID NO: 59), Aβ₁₇₋₄₂ (SEQ ID NO: 58), Aβ₁₋₄₂ (SEQ ID NO: 60) and a non-relevant 38-mer peptide (p1412). Cultures were incubated at 37° C. with 5% CO₂ for 72 hours, and then 100 μL of supernatant was removed from each well and frozen at −70° C. for cytokine analysis. Ten L of culture medium containing 0.5 Ci of ³H-thymidine (³H-TdR, Amersham, Cat No. TRK637) was added to each well and incubated for 18 hr, followed by detection of radioisotope incorporation by liquid scintillation counting. The mitogen phytohemagglutinin (PHA) was used as a positive control for lymphocyte proliferation. Cells cultured alone without Aβ peptide or PHA mitogen were used as the negative and positive controls. The stimulation index (SI) was calculated as mean counts per min (cpm) of triplicate experimental cultures with Aβ peptide divided by mean cpm of triplicate negative control cultures; a SI>3.0 was considered a significant proliferation response.

a. Proliferation Analysis

Peripheral blood mononuclear cell samples were isolated from whole blood collected at week 0 (baseline) and week 16 (4 weeks after the third dose) from patients with Alzheimer's Disease vaccinated with UB-311 vaccine and then cultured in the absence or presence of various Aβ peptides. As shown in Table 13, no significant proliferation response by lymphocytes was observed when Aβ₁₋₁₄, other Aβ peptides, or p1412 (a non-relevant control peptide) were added to the culture medium. As expected, positive proliferation responses were noted when PHA mitogen was added to culture medium. The observation of similar responses to PHA before and after UB-311 immunization (p=0.87) suggests no significant alteration in study subjects' immune functions (Table 13).

Statistical Analysis. The differences in lymphocyte proliferation between week 0 and week 16 were examined by the paired t-test. Statistical significance levels were determined by 2-tailed tests (p<0.05). R version 2.14.1 was used for all statistical analyses.

b. Cytokine Analysis

Cytokine analyses (IL-2, IL-6, IL-10, TNF-α, IFN-γ) from the PBMC cultures were performed on aliquots of culture medium with cells alone or in the presence of Aβ peptide domains or PHA. Human-specific cytokine sandwich ELISA kits (U-CyTech Biosciences, Utrecht, The Netherlands) were used to determine the concentrations (pg/mL) of individual cytokines following the manufacturer's instructions (Clin Diag Lab Immunol. 5(1):78-81 (1998)).

The PBMC samples collected from Alzheimer's Disease patients receiving UB-311 vaccine at week 0 and week 16 were also tested for cytokine secretion either with cells alone (negative control) or in the presence of Aβ peptides, p1412 (non-relevant peptide) or PHA mitogen (positive control) after being cultured for 3 days. The quantifiable range of the kit is between 5 and 320 μg/mL. Any measured concentration below 5 μg/mL or above 320 μg/mL was indicated as below quantification limit (BQL) or above quantification limit (AQL), respectively. However, for statistical considerations, BQL or AQL was replaced with the lower (5 μg/mL) or upper (320 μg/mL) quantifiable limit, respectively. The mean concentrations of each cytokine at week 0 and week 16 are shown in Table 14. As expected, there were significant increases in cytokine production in the presence of PHA, the positive control, except for IL-2. The production of cytokines in response to the stimulation with Aβ₁₋₁₄, or other Aβ peptides was observed at baseline (week 0) and week 16, but most values appeared similar to the corresponding negative controls (cells alone).

In order to assess the change of cell-mediated immune response after immunization, the change of mean cytokine concentrations from baseline to week 16 was compared with that of the negative controls and examined by paired Wilcoxon signed-rank test. Four cytokines (IFN-γ, IL-6, IL-10, TNF-α) showed notable increase in secretion in response to full-length Aβ₁₋₄₂ peptide; this observation may be due to the conformational epitopes of Aβ₁₋₄₂ aggregates. Up-regulation of cytokine secretion was not detected in Aβ₁₋₁₄ or other Aβ peptides.

c. Summary

UB-311 vaccine contains two peptide immunogens each with a N-terminal Aβ₁₋₁₄ peptide synthetically linked to MvF5 Th and HBsAg3 Th epitopes respectively. In vitro lymphocyte proliferation and cytokine analysis were used to evaluate the impact of immunization of UB-311 vaccine on the cellular immune response. No proliferation responses by lymphocytes were observed when the Aβ₁₋₁₄ peptide or any other Aβ peptides was added to culture medium as shown in Table 13. Up-regulation of cytokine secretion by lymphocytes of UB-311 vaccine-immunized patients was not detected upon treatment with the Aβ₁₋₁₄ and other Aβ peptides except for Aβ₁₋₄₂, which elicited appreciable increase of four cytokines (IFN-γ, IL-6, IL-10, TNF-α) after UB-311 immunization at week 16 when compared to week 0 levels before treatment (Table 14). The increase of cytokine release through Th2 type T cell response is more likely unrelated to the UB-311 vaccine response since no up-regulation detected with Aβ₁₋₁₄ alone. The response to Aβ₁₋₄₂ is suspected to be a background response to native Aβ that may be related to native T helper epitopes identified on Aβ₁₋₄₂. The lack of IL-2 production in response to PHA was observed, which is consistent with the findings reported by Katial R K, et al. in Clin Diagn Lab Immunol 1998; 5:78-81, under similar experimental conditions with normal human PBMC. In conclusion, these results showed that the UB-311 vaccine did not generate potentially inflammatory anti-self, cell-mediated immune responses in patients with mild to moderate Alzheimer's disease who participated in the phase I clinical trial, thus further demonstrating the safety of the UB-311 vaccine.

### Example 9

### Promiscuous Artificial Th Responsive Cells can be Detected in Naïve Peripheral Blood Mononuclear Cells (PMBC) in Normal Blood Donors with Moderate Immunogenic Inflammatory Response when Compared to Negative Control

ELISpot Assay was employed to detect promiscuous artificial Th responsive cells in naïve peripheral blood mononuclear cells in normal blood donors to assess their potency to elicit inflammatory responses when compared to a potent mitogen Phytohemagglutinin (PHA) and negative control.

ELISpot assays employ the sandwich enzyme-linked immunosorbent assay (ELISA) technique. For detection of T cell activation, IFN-7 or related cytokine was detected as an analyte. Either a monoclonal or polyclonal antibody specific for the chosen analyte was pre-coated onto a PVDF (polyvinylidene difluoride)-backed microplate. Appropriately stimulated cells were pipetted into the wells and the microplate was placed into a humidified 37° C. CO₂ incubator for a specified period of time. During this incubation period, the immobilized antibody, in the immediate vicinity of the secreting cells, bound to secreted analyte. After washing away any cells and unbound substances, a biotinylated polyclonal antibody specific for the chosen analyte was added to the wells. Following a wash to remove any unbound biotinylated antibody, alkaline-phosphatase conjugated to streptavidin was added. Unbound enzyme was subsequently removed by washing and a substrate solution (BCIP/NBT) was added. A blue-black colored precipitate formed and appeared as spots at the sites of cytokine localization, with each individual spot representing an individual analyte-secreting cell. The spots were counted with an automated ELISpot reader system or manually, using a stereomicroscope.

In the in vitro study conducted, PHA at 10 μg/mL culture was used as a positive control. UBITh®1 (SEQ ID NO: 17) and UBITh®5 (SEQ ID NO: 6) peptides were tested for the number of responsive cells present in the peripheral blood mononuclear cells in regular normal blood donors. A mixture of promiscuous artificial Th epitope peptides with SEQ ID NOs: 33 to 52 were prepared as another positive control. Media alone was used as the negative control in a standard T cell stimulation cell culture condition. Briefly, 100 μL/well of PBMCs (2×10⁵ cells) stimulated with mitogen (PHA at 10 μg/mL), or Th antigen (UBITh®1, UBITh®5 or mixture of multi-Ths at 10 μg/mL) were incubated at 37° C. in a CO₂ incubator for 48 hours. The supernatant from wells/plates were collected. The cells on the plates were washed and processed for detection of the target analyte, IFN-7.

As shown in FIG. 15, representative donors 1, 2, and 3 were tested for their responsive cells to promiscuous artificial UBITh®1 or UBITh®5 epitope peptides. An overwhelming IFN-7 ELISPOT number was always detected with naïve donor (PBMCs cultivated with PHA; too numerous to count) while PBMCs cultivated with control media gave a background IFN-7 ELISPOT number between 5 to 50. Moderate ELISPOT numbers were detected for naïve donor PBMCs cultivated with UBITh®1 or UBITh®5 from 20 to around 120. A mixture of multi Th peptides with SEQ ID NOs: 33-52 was also cultivated with naïve donor PBMCs for comparison with ELISPOT numbers come in from 20 to about 300 as expected. Such stimulatory responses triggered by the UBITh®1 or UBITh®5 peptides are about 3 to 5 times compared to the negative controls.

In summary, promiscuous artificial Th responsive cells can be readily detected in naïve donor PBMCs which stand ready to mount immune responses to help the B cell antibody production and the corresponding effector T cell responses by secreting signature cytokines. IFN-γwas used as one example here to illustrate this stimulatory nature of these Th epitope peptides. However, such stimulatory inflammatory responses are moderate enough to mount a suitable effector cell responses (B cell for antibody production, cytotoxic T cells for killing of target antigenic cells) so as not to cause untoward inflammatory pathophysiological responses during a vaccination process.

### Example 10

### Neo-Epitope Based Peptide Immunogen Constructs and Formulations Thereof for Personalized Cancer Immunotherapy

We are in the midst of a T cell revolution in cancer treatment. In the past several years, new therapies like immune checkpoint inhibitors (ICI) and adoptive cell therapies like chimeric antigen receptors (CAR-Ts) have offered new hope to millions of people suffering from cancer. ICI drugs help overcome natural barriers erected by cancers to prevent our own immune system, and in particular, our T cells, from fighting cancer. For example, CAR-T therapy engineers the T cells of a patient to mount an immune response against certain tumor cells. These new therapies bring future therapies designed to “educate” T cells to attack tumors with ever greater accuracy.

There is growing excitement around personalized, or neoantigen, cancer vaccines as a way to offer new hope to millions of people suffering from cancer. Neoantigens are personalized tumor mutations that are seen as foreign by the immune system of most individuals. A personalized vaccine, therefore, targets these neoantigens, which educate the immune system to find and kill the tumor.

Bioinformatic, proteomic, cell based assays and non-standard methods have been widely applied for efficient identification of true neoantigens. RNA frameshift (FS) variants formed by INDELs, i.e., short INsertions and DELetions, in microsatellites and mis-splicing of exons are a rich source of highly immunogenic neoantigens.

Arrays containing all possible (e.g., 400K) FS (frameshift) tumor derived peptides can be applied in detection of antibody reactivity from one drop of patient blood, thus offering a remarkable tool for cancer diagnosis and identification of neoantigen CTL epitopes.

MHC typing could be performed using RNAseq, while NetMHC and NetMHCpan could be used to predict the neoentigen binding affinity.

An in vitro HLA agnostic assay, polypeptides representing each identified mutation from a patient's tumor are delivered individually into their own antigen presenting cells (APCs), which are then processed and presented the peptides on the cell's surface where they are recognized by various effector T cells. If a T cell recognizes and binds to the peptide, a cytokine response will be triggered. A true antigen is measured and determined to be “good” (i.e., or stimulatory) or “bad” (i.e., inhibitory). The actual antigens to which a patient's T cells—both CD4⁺ (helper T cells) and CD8⁺ (killer T cells) respond can be identified and selected as Neo-epitope for design of neo-epitope peptide immunogen constructs.

By including empirically confirmed neo-antigens to which patients have pre-existing responses, a personalized cancer vaccine to which patients' immune systems are already primed is therefore developed.

Briefly, a neo-epitope peptide derived from a specific empirically confirmed neoantigen comprising a B or CTL neo-epitope can be rendered highly immunogenic employing the design principles as described in the present disclosure. With the participation of the promiscuous artificial Th epitopes covalently linked to a selected neo-epitope, such neo-epitope peptide immunogen construct can facilitate the induction and maintenance of antibodies directed to the neo-antigens and induction of effector CTL functions as well as the generation of B and CTL memory cells, leading to sustained B and CTL responses and a robust antitumor immunity.

Representative public neoantigens with selected target epitope sequences for melanoma neoantigens, Histone3 Variant H3.3K27M for Glioma, and KRAS (mutant with G to D) for Colorectal Cancer are shown as SEQ ID NOs: 73, 74, and 75, respectively, in Table 3A.

The designed neo-epitope peptide immunogen constructs and formulations thereof of the present disclosure can be further evaluated in clinical trials for the safety, immunogenicity, and efficacy that consists of three parts:
1) A study of the safety and immunogenicity as monotherapy in cancer patients with no evidence of disease but at high risk of relapse.
2) A study of the safety, immunogenicity, and efficacy of the neo-epitope vaccine in combination with an FDA-approved immune checkpoint inhibitor in patients with advanced or metastatic solid tumors.
3) A study of the safety, immunogenicity, and efficacy of the neo-epitope vaccine as monotherapy in patients with relapsed or refractory solid tumors who have failed to respond to or whose cancer has progressed after treatment with an immune checkpoint inhibitor.

Eligible patients having completed their treatment (e.g., Surgical resection, neoadjuvant and/or adjuvant chemotherapy, and/or radiation therapy) for cutaneous melanoma, non-small cell lung cancer (NSCLC), squamous cell carcinoma of the head and neck (SCCHN), or urothelial carcinoma and show no evidence of disease by CT or MRI can participate in these cancer immunotherapy.

### Example 11

### Tumor-Associated Carbohydrate Antigens (TACA)-B Epitope Immunogen Constructs and Formulations Thereof for Cancer Immunotherapy

Tumor cells are characterized by aberrant glycosylation patterns that result in heterogeneity, truncation and overexpression of surface oligosaccharides. Three major categories of saccharide structures have been identified as potential Tumor-Associated Carbohydrate Antigens (TACAs) shown as GD3, GD2, Globo-H, GM2, Fucosyl GM1, PSA, Le^{y}, Le^{x}, SLe^{x}, SLe^{a}, and STn as shown in FIG. 16.
(1) Mucin-relative o-glycan: Tn, TF and STn
(2) Glyco-sphingolipids: including ganglioside GM3, GM2, GD2, GD3, fucosyl GM1 and neutral globoside globol H
(3) Blood-group antigens: SLe^{x}, Le^{y}Le^{x}, SLe^{a} and Le^{y}

Globol H is expressed on the breast cancer cell surface as a glycolipid and is an attractive tumor marker. Globol H hexa-saccharide was synthesized using the glycal assembly approach to oligosaccharide synthesis. The Globa H serves as a B-hapten and can be armed with a functional group at the reducing ends to allow covalent immobilization with the Th helper peptides shown in Table 2 to form immunogenic peptide constructs. The use of the disclosed Th epitopes in this type of application is much more versatile than other approaches using KLH or other conventional carrier proteins as described previously by Danishefsky and Livingston (website: glycopedia.eu/Hetero-TACA-vaccines-based-on-protein-carriers).

The N-terminus of each polypeptide or primary amines in the side chain of lysine (K) residues of proteins are available as targets for N-hydroxysuccinimide (NHS) type of crosslinking linker reagents at pH 7-9 to form stable amide bonds, along with the release of the N-hydroxysuccinimide leaving group. In addition, m-maleimidobenzoyl-N-hydroxysuccinimide ester (MBS) (H. L. Chiang, et al. Vaccine. 2012 30(52), 7573-7581), p-nitrophenyl ester (PNP) (S. J. Danishefsky, et al. Acc. Chem. Res. 2015, 48(3), 643-652) along with different chain length linkers can also serve as important TACA conjugation linkers to the Th helper peptides shown in Table 2.

The artificial Th epitopes of the present disclosure can be used in a cancer vaccine composition comprising: (a) immunogenic composition comprising a glycan essentially of Globol H or an immunogenic fragment thereof, (b) an immunogenic fragment that is covalently linked to a promiscuous artificial T helper epitope peptide shown in Table 2. Th epitope peptides shown in Table 2 (e.g., UBITh®) are individually prepared stepwise by solid phase peptide synthesis (SPPS) and Fmoc chemistry with deprotection/coupling from the C-terminus to N-terminus. The target peptide sequence was constructed accordingly.

The glycan can be linked to the artificial Th epitope peptide, with or without a spacer, through amide bond formation directly on the solid phase resin with the coupling reaction being monitored by the Kaiser test. Two strategies can be utilized for this coupling: (1) to prepare glycan derivatives with an activating leaving group followed by coupling with the resin bound spacer linked Th peptide where the N-terminus has a free amine or (2) the conversion of the resin bound N-terminal amine group to become an activating ester followed by a coupling reaction with the glycan. The direct coupling of glycan to a resin-bound and spacer-Th peptide would allow a more efficient coupling reaction to the activating group on the resin-bound free amine group followed by the release of the coupled glycan peptide from the resin by standard resin free cleavage reaction. The steric hindrance between two free large molecules, such as polysaccharides and long chain peptide, would make the glycan-peptide coupling reaction more difficult and, thus, less efficient and with low yield.

In some aspects, the B-hapten-linked T helper carrier is the spacer linked Th-peptides described in Table 2.

In some embodiments, the linker is a p-nitropheny linker, a N-hydroxysuccinimide linker, a p-nitrophenyl ester (PNP ester), or a N-hydroxysuccinimide ester (NHS ester). NHS esters can react with primary amines at pH 7-9 to form stable amide bonds, along with the release of the N-hydroxysuccinimide leaving group.

The following abbreviations are used in this Example: PNP: p-nitrophenyl ester; NPC: N-nitrophenyl chloroformate; DSS (disuccinimidyl suberate); and NHS: N-hydroxysuccinimide; MBS: m-maleimidobenzoyl-N-hydroxysuccinimide ester.

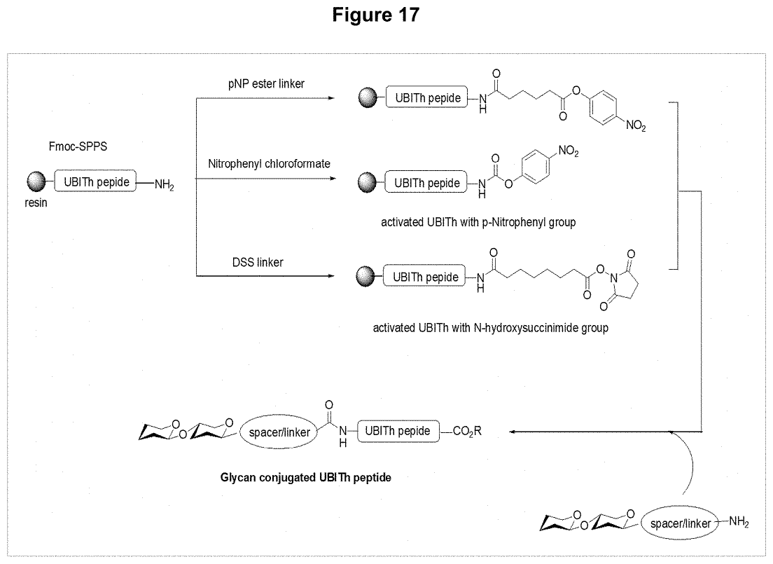

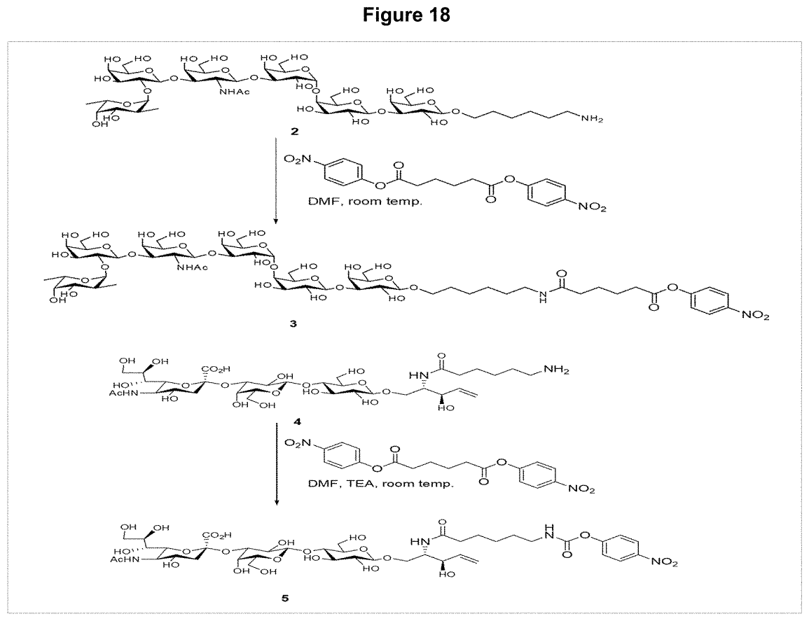

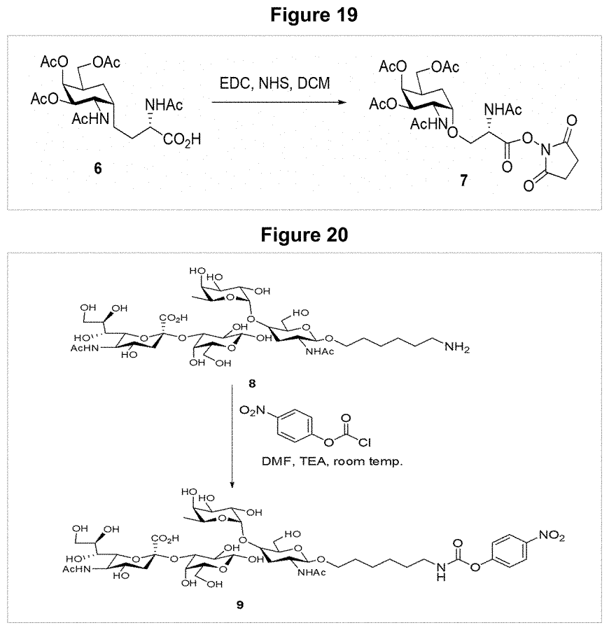

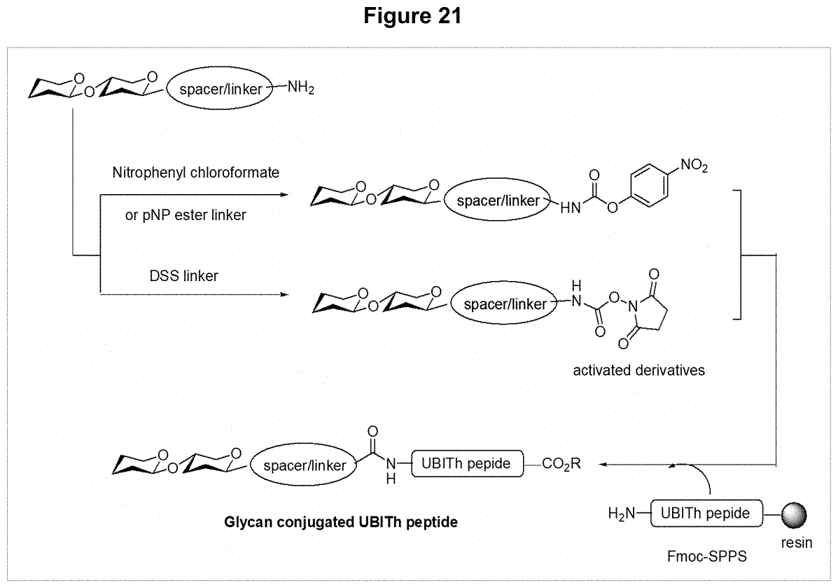

The following steps of chemical reactions and preparations are included (FIGS. 17 to 21) to demonstrate various embodiments.

1. Preparation of Activated UBITh® with p-Nitrophenyl (PNP) Group

An artificial Th epitope (e.g., UBITh®) carrier can be synthesized by automated solid-phase synthesis and Fmoc chemistry. Following the Fmoc-deprotection of N-terminal amino acid on the elongation peptide chain, the free amino group can be converted to an active 4-nitrophenyl group by treating with 4-nitrophenyl chloroformate (NPC, 10 eq) in DMF solution containing 10% triethylamine. The resulting peptide-resin mixture can be wash with DCM solution to remove reagent and 4-ntriophenol residue. The desired activated UBITh® with p-Nitrophenyl group can be obtained for further conjugation with glycan.

2. Preparation of Activated UBITh® with N-Hydroxysuccinimide (NHS) Group

UBITh® peptide carrier can be synthesized using automated solid-phase synthesis and Fmoc chemistry. Following the Fmoc-deprotection of N-terminal amino acid on the elongation peptide chain, the free amino group can be converted to an active N-hydroxysuccinimide by treating with DSS in DMF solution. The resulting peptide-resin can be wash with DCM solution to remove reagent residue. The desired activated UBITh® with N-hydroxysuccinimide group can be obtained for further conjugation with glycan.

3. Synthesis of Globol H Conjugated UBITh® Peptide

The preparation of Globol H hexasaccharide analogs with terminal amine group (2) can be achieved by following the one-pot synthesis strategy (C. Y Huang, et al., Proc. Natl Acad Sci USA 2006, 103, 15-20). Briefly, a solution of Globol H can be introduced into the activated UBITh®-resin and then gently mixed for 3 h. Following cleavage from the resin and full deprotection, the crude Globol H conjugated UBITh® peptide can be purified by preparative high performance liquid chromatography (HPLC), and characterized by matrix-assisted laser desorption ionization-time of flight (MALDI-TOF) mass spectrometer and reverse-phase HPLC analysis.

4. Preparation of Globol H Activated Ester

Globol H hexylamine (2) can be dissolved in anhydrous DMF solution. p-Nitrophenyl adipate diester can then be added and stirred for 2-4 hours at room temperature. The reaction can be monitored with TLC and Kaiser Test to check disappearance of the free amine group. The DMF solvent can be removed under reducing pressure without heating and then resulting residue can be extracted with dichloromethane and water with 0.5% acetic acid three times. The resulting aqueous solution can be concentrated and purified by reverse phase column (RP-C18) chromatography (isocratic elution with MeOH/H₂O with 1% acetic acid).

5. Preparation of GM3 Activated Ester

The synthesis and purification of GM3 ganglisides analogs have been previously described (Jacques S, et al., J. Am. Chem. Soc., 2012 134(10):4521-4) The GM3 analogs amine X (4.5 mg; 5.2 μmol) can be dissolved in dimethylformamide (1.5 mL), and triethylamine (3.0 eq.) added. p-Nitrophenyl adipate diester (10.0 eq.) can then be added. The reaction can be complete as monitored by TLC (CH₂Cl₂-MeOH-H₂O-AcOH; 4:5:1:0.5). The pH of the reaction can be adjusted to 5.0 with acetic acid and followed by co-evaporated with toluene (3 x). The residue can be concentrated and the residue obtained can be purified by HPLC (Beckman C18-silica semi-preparative column) using a gradient of MeOH-H₂O containing 1% acetic acid. The 1H NMR spectrum can be acquired in CD₃OD.

6. Preparation of Tn Activated Ester

The saccharide Tn can be synthesized based on the previous reports (T. Toyokuni, et al., Bioorg Med Chem. 1994, 11, 1119-32; and S. D. Scott, et al., J. Am. Chem. Soc., 1998, 120(48), 12474-85). Tn analog 6 (mmol), NHS (160 mg, 1.39 mmol), and EDC (268 mg, 1.40 mmol) in dry CH₂Cl₂ (25 mL) can be stirred at room temperature for 1 hour. The mixture can be washed with precooled H₂O (3×30 mL), dried (Na₂SO₄), and concentrated to give the succinimide-ester derivatives (7) as a colorless syrup.

7. Preparation of Sialyl Lewis x (sLe^{x}) Active Ester (9)

The preparation of sLe^{x} tetrasaccharide derivative (8) can be achieved with the published synthetic strategy (G. Kuznik, Bioorganic & Medicinal Chemistry Letters, 7(5):577-580, 1997). Compound (8) added to a solution of NPC (2 eq.) in DMF/DCM armed up to room temperature and stirred for another 30 mins. The concentrated crude mixture can be washed with DCM and aqueous solution. The resulting aqueous solution can be treated under reducing pressure and purified with RP C18 column.

8. General Procedure of Generating Glycocoiunates

Standard coupling reaction of individual glycans onto individual resin bound spacer-incorporated Th peptides follows the standard peptide bond formation coupling procedures administered by regular solid phase peptide synthesizer(s) via carbodiimide coupling reaction.

The resin bound glycan-peptide can be removed from the resin and recovered and precipitated and lyophilized according to standard peptide synthesis procedure.

T-cell peptide epitopes (e.g., UBITh®) carrier can be synthesized by automated solid-phase synthesis and Fmoc chemistry. Following the Fmoc-deprotection of N-terminal amino acid on the elongation peptide chain, the free amino group can be made available for a further conjugation reaction. Synthetic saccharide analogs of Globo H, Tn, GM3, SLe^{x}, etc. with activating leaving group modification can be dissolved in DMF solution and added into the SPPS system for reaction with N-terminal amine of UBITh® peptide. The reaction can be monitored with a Kaiser test. Following cleavage from the resin and full deprotection, the saccharides conjugated UBITh® peptide can be purified by preparative high performance liquid chromatography (HPLC), and characterized by matrix-assisted laser desorption ionization-time of flight (MALDI-TOF) mass spectrometer and reverse-phase HPLC analysis.

In summary, in this Example, effective conjugation of individual glycans to individual Th helper epitope peptides shown in Table 2 are described in detail to allow such carbohydrate-peptide immunogen constructs to be efficiently prepared for subsequent vaccine formulations. These carbohydrate vaccine formulations using the artificial Th epitopes could provide focused antibody responses directed to the targeted carbohydrate antigens, typically present on cancer cells to allow immunotherapy of cancer patients in clinical protocols.

### Example 12

### Enhancement of Effector T Cell Functions by Linkage of Promiscuous Artificial T Helper Epitope(s) to the Effector Cell Epitopes (e.g., CTL Epitopes) and Formulations Thereof for the Development of Universal T Cell Vaccines Against Viral Infections

### Introduction

T-helper cells carry the surface marker CD4 and express a surface receptor known as the T cell receptor composed of a polypeptide heterodimer (designated e.g., a/0). T helper cells recognize viral peptides in association with class II MHC protein, usually on the surface of an antigen-presenting cell (APC). These interactions result in T helper cell activation, proliferation and differentiation, providing sufficiently high binding affinity.

T Helper cells (CD4⁺ T cells) provide soluble mediators and receptor-ligand interactions to cells of both the innate and the adaptive immune systems that trigger and modulate their effector function. These cells are a heterogenous population and, to date, several subsets have been characterized including: Th1, Th2, Th17, and T follicular helper (Tfh) cells. There are also regulatory CD4⁺ T cells (Tregs) that repress the growth and function of T cell helper and cytotoxic subsets.

Each type of effector T cell is controlled by a key transcriptional regulator, expresses a distinct array of cell surface molecules, and secretes “signature” cytokines, which together facilitate the specific role of that T cell subset within an arm of the immune system.

Tfh cells are distinguished from other helper subsets by their unique ability to home to B cell follicles and provide help to antigen-specific B cells that are undergoing somatic hypermutation (SHM) of their Ig V region genes and changing their affinity for antigen. Tfh cells secrete the cytokine IL-21 that is essential for B cell differentiation and the development of high-affinity, isotype-switched antibody responses against viruses. Tfh-mediated signals ensure selection of B cells with higher affinity to the immunizing antigen, which can then differentiate to become long-lived plasma cells or memory B cells. Because of the possibility of the emergence of self-reactive B cell clones through the process of SHM and the longevity of selected clones, it is paramount that stringent tolerance mechanisms exist to control delivery of positive selection signals from Tfh cells to B cells.

Th1 cells are principally involved in boosting the cytotoxic response. These cells promote the cell-mediated response to virus infection by stimulating the maturation of cytotoxic T cell precursors, partly through the secretion of the cytokines IL-2 and IFN-γ. Th1 cells also secrete tumor necrosis factor (TNF), mediate delayed-type hypersensitivity reactions, and promote the production of IgG2a antibodies. Th1 cells greatly augment the immune response by activating macrophages and other T cells at the site of the viral infection. This response is the basis for delayed-type hypersensitivity reactions that are a recognized part of the pathogenesis of many viral infections.

Other Th cells, including Th2 and Th17 cells, also contribute to the immune response against virus infection by promoting inflammation or the generation of specific antibody isotypes.

Some T cells can downregulate other T cell and/or B cell responses. A distinct subset of CD4⁺ T cells known as regulatory T cells (T-regs). There are two basic types of T-regs: (1) tTregs that are produced in the thymus during negative selection and are thought to be mainly involved in controlling autoimmune disease; and (2) iTregs that are induced during immune responses and are involved in terminating immune responses and bringing the immune system back to homeostasis. T-reg cells may also help maintain the balance between protection and an immune-mediated pathology.

The impact of helper T cells in peptide vaccination is tremendous. CD4⁺ T helper cells, upon activation, can provide strong sustainable CD8⁺ T-cell responses through the inclusion of cognate T helper epitopes. In view of the local immunomodulatory function of CD4 T cells, it is preferred to activate cognate help in the target tissue derived from antigens of the targeted virus or tumor. Foreign antigens and even tumor-associated proteins often contain immunogenic stretches (Th epitopes) that function as hotspots for the immune system. Peptide immunogen construct as designed and described in this instant invention through covalent linkage of selected promiscuous artificial Th epitopes to target B or effector T cell (e.g., CTL epitopes) can facilitate intimate interactions of APC, CD4, and CD8 T cells for the induction of optimal and protective CD8 T-cell responses.

Examples of CTL Epitope Peptides as Target Antigenic Sites for Incorporation into Viral Specific Universal T Cell Vaccines

1. HIV CTL Vaccine Component:

Despite antiretroviral therapy (ART), human immunodeficiency virus (HIV)-1 persists in a stable latent reservoir, primarily in resting memory CD4⁺ T cell. This reservoir presents a major barrier to the cure of HIV-1 infection. To purge the reservoir, pharmacological reactivation of latent HIV-1 has been tested both in vitro and in vivo. A key remaining question is whether virus-specific immune mechanisms, including cytotoxic T lymphocytes (CTLs), can clear infected cells in ART-treated patients after latency is reversed. After extensive data mining, CTLs that could recognize epitopes from unmutated latent HIV-1 in every chronically infected patients tested were identified with specific peptides representative of such CTL epitopes as shown in Table 3B (SEQ ID NOs: 76-82) being incorporated into the design of a universal HIV T cell vaccine. Chronically infected patients retain a broad-spectrum viral-specific CTL response. It is anticipated that appropriate boosting of this response through this HIV universal T cell vaccine incorporating these CTL epitope peptides with covalent linkage individually to a promiscuous artificial Th epitopes of this invention (SEQ ID NOs: 1-52) would result in the elimination of the latent reservoir.

2. HSV CTL Vaccine Component

Herpes simplex virus infects a high percentage of the world population and establishes a latent infection in which the viral genome is retained in sensory neurons, but no virions are produced. Periodic reactivation of the virus from this latent state results in lesions that can affect the mucosal surfaces of the mouth and lips, genital tract, and cornea of the eye, and less frequently the skin and brain. HSV-2 can be lethal to newborns who acquire it from the birth canal; corneal HSV-1 infections are a leading infectious cause of blindness; and brain HSV-1 infections account for approximately one quarter of cases of viral encephalitis that can be fatal. HSV-1 vaccines that have made their way to clinical trials have been primarily designed for Ab production and have been largely ineffective. Evidence suggests a significant role for CD8⁺ T cells in controlling HSV infections in both mice and humans.

HSV type 1 (HSV-1) expresses its genes sequentially as immediate early (a), early (3), leaky late (71), and true late (γ2), where viral DNA synthesis is an absolute prerequisite only for 72 gene expression. The 71 protein glycoprotein B (gB) contains a strongly immunodominant CD8⁺ T cell epitope (gB₄₉₈₋₅₀₅) that is recognized by 50% of both the CD8⁺ effector T cells in acutely infected trigeminal ganglia (TG) and the CD8⁺ memory T cells in latently infected TG.

Through an extensive data mining and thorough data analyses, an entire HSV-specific CD8⁺ T cell repertoire in C57BL/6 mice was included for HSV CTL vaccine design consideration. Furthermore, different sets of HSV-1 gB epitopes were found to be recognized by CD4⁺ T cells from symptomatic versus asymptomatic individuals. Among these, gB₁₆₆₋₁₈₀, gB₆₆₁₋₆₇₅, and gB₆₆₆₋₆₈₀ were targeted by CD4⁺ CTLs that lysed autologous HSV-1—and vaccinia virus (expressing gB [VVgB])-infected LCLs. gB₁₆₆₋₁₈₀ and gB₆₆₆₋₆₈₀ appeared to be recognized preferentially by CD4⁺ T cells from HSV-1-seropositive healthy “asymptomatic” individuals, while gB₆₆₁₋₆₇₅ appeared to be recognized preferentially by CD4⁺ T cells from severely “symptomatic” individuals. An effective immunotherapeutic herpes vaccine would exclude the potential “symptomatic” gB₆₆₁₋₆₇₅ epitope. In addition, three VP11/12 CD8⁺ epitopes that are highly recognized in asymptomatic individuals were also identified that were found to elicit a strong protective immunity in the “humanized” HLA-A*02:01 transgenic mouse model of ocular herpes.

A series of HSV CTL epitopes were identified with specific peptides representative of such CTL epitopes as shown in Table 3B (SEQ ID NOs: 83-106) being incorporated into the design of this instant invention for the development of a universal multiepitope based HSV T cell vaccine.

3. FMDV, PRRSV, and CSFV Universal T Cell Vaccines in the Swine Industry

Foot-and-mouth disease virus (FMDV), porcine reproductive and respiratory syndrome virus (PRRSV) and classical swine fever virus (CSFV) are debilitating pathogens in the swine industry. The development of effective vaccines against these pathogens is of practical significance in the swine industry.

Although neutralizing antibodies induced upon vaccination are highly effective in controlling disease and viral transmission, they do not confer cross-subtype protection and might become ineffective due to antigenic changes. Cellular immune responses, especially production of cytotoxic T lymphocytes (CTL), are receiving much attention due to their potential in developing efficient and cross-protective peptide vaccines against various viruses. For example, the CTL epitope peptides could be used for the development of cross-protective human influenza vaccines, including recombinant viral vector and peptide vaccine; the CTL epitope peptide identified for FMDV serotype O was cross-reactive to other FMDV serotypes. However, most of the analyses were restricted to specific viral proteins and were only able to identify few CTL epitopes.

After extensive data mining, design, synthesis, labor-intensive and time-consuming immunogenicity and functional assay procedures, assessment of large sets of designer CTL peptides have allowed validation of selected viral specific CTL epitopes derived from various FMDV, PRRSV, and CSFV viral proteins. Selected CTL peptides representative of these epitopes are shown in Table 3B with SEQ ID NOs: 107-145.

In our selection and identification process, a bioinformatics pipeline was integrated for analysis of swine viral sequences to resolve several challenges: (1) genetic variation, (2) incomplete screening from particular surface proteins, and (3) inappropriate prediction based on non-swine leukocyte antigens. In corporation of these CTL epitope peptides with proper linkage of promiscuous artificial Th epitope peptides resulting in peptide immunogen constructs of this instant invention would lead to the development of T cell vaccines with sustained memory and long duration CTL responses. The commercial development of such high precision efficacious peptide based swine vaccines against FMDV, PRRSV, CSFV and other viral infections which frequently devastate the swine industry would be of paramount importance to the husbandry industry.

TABLE 1
Amino Acid Sequences of Pathogen Protein Derived Th Epitopes Including
Idealized Artificial Th Epitopes for Employment in the Design of
Peptide Immunogen Constructs

| Description | Sequence | SEQ ID NO |
|---|---|---|
| MvF Th | DLSDLKGLLLHKLDGL | 1 |
| (SSAL1 Th1) | EI EIR III RIE I | 2 |
| | V V VVV V V | 3 |
| | F F FFF F F | 4 |
| | XXSXXXGXXXHXXXGX | 5 |
| MvF1 Th (UBITh ®5) | LSEIKGVIVHRLEGV | 6 |
| MvF2 Th | ISEIKGVIVHKIEGI | 7 |
| | ISISEIKGVIVHKIEGILF | 8 |
| MvF3 Th | T RT TR T | 9 |
| | ISIXEIXXVIVXXIEXILF | 10 |
| KKKMvF3 Th | KKKISISEIKGVIVHKIEGILF | 11 |
| | T RT TR T | 12 |
| | KKKISIXEIXXVIVXXIEXILF | 13 |
| | ISISEIKGVIVHKIETILF | 14 |
| MvF4 Th (UBITh ®3) | T RT TR | 15 |
| | ISIXEIXXVIVXXIETILF | 16 |
| MvF5 Th (UBITh ®1) | ISITEIKGVIVHRIETILF | 17 |
| KKKMVF5 Th (UBITh ®1a) | KKKISITEIKGVIVHRIETILF | 18 |
| | KKKLFLLTKLLTLPQSLD | 19 |
| | RRRIKII RII I L IR | 20 |
| HBsAgl Th | VRVV VV V I V | 21 |
| (SSAL2 Th2) | F FF FF F V F | 22 |
| | F | 23 |
| | XXXXXXXTXXXTXPXSXX | 24 |
| HBsAg2 Th | KKKIITITRIITIPQSLD | 25 |
| | FFLL L ITTI | 26 |
| | KKKXXXXTRIXTIXXXXD | 27 |
| HBsAg3 Th (UBITh ®2) | KKKIITITRIITIITTID | 28 |
| HBsAg Th (UBITh ®4) | FFLLTRILTIPQSLD | 29 |
| KKK-HBsAg Th | KKKFFLLTRILTIPQSLD | 30 |
| HBsAg Th | FFLLTRILTIPQSL | 31 |
| Bordetella pertussis Th (UBITh ®7) | GAYARCPNGTRALTVAELRGNAEL | 32 |
| Cholera Toxin Th | ALNIWDRFDVFCTLGATTGYLKGNS | 33 |
| Clostridium tetani TT1 Th | QYIKANSKFIGITEL | 34 |
| Clostridium tetani1 Th (UBITh ®6) | KKQYIKANSKFIGITEL | 35 |
| Clostridium tetani TT2 Th | FNNFTVSFWLRVPKVSASHLE | 36 |
| Clostridium tetani TT3 Th | KFIIKRYTPNNEIDSF | 37 |
| Clostridium tetani TT4 Th | VSIDKFRIFCKALNPK | 38 |
| Clostridium tetani2 Th | WVRDIIDDFTNESSQKT | 39 |
| Diphtheria Th | DSETADNLEKTVAALSILPGHGC | 40 |
| EBV BHRF1 Th | AGLTLSLLVICSYLFISRG | 41 |
| EBV EBNA-1 Th | PGPLRESIVCYFMVFLQTHI | 42 |
| EBV CP Th | VPGLYSPCRAFFNKEELL | 43 |
| EBV GP340 Th | TGHGARTSTEPTTDY | 44 |
| EBV BPLF1 Th | KELKRQYEKKLRQ | 45 |
| EBV EBNA-2 | TVFYNIPPMPL | 46 |
| HCMV IE1 Th | DKREMWMACIKELH | 47 |
| Influenza MP1_1 Th | FVFTLTVPSER | 48 |
| Influenza MP1_2 Th | SGPLKAEIAQRLEDV | 49 |
| Influenza NSP1 Th | DRLRRDQKS | 50 |
| Plasmodium falciparum Th | DHEKKHAKMEKASSVFNVVNS | 51 |
| Schistosoma mansoni Th | KWFKTNAPNGVDEKHRH | 52 |

TABLE 2
Examples of Optional Heterologous Spacers and CpG Oligonucleotides

| | | SEQ |
|---|---|---|
| Description | Sequence/Composition | ID NO |
| Naturally- | Naturally-occurring amino acids include: | N/A |
| Occurring Amino Acids | alanine, arginine, asparagine, aspartic acid, cysteine, glutamic | |
| | acid, glutamine, glycine, histidine, isoleucine, leucine, lysine, | |
| | methionine, phenylalanine, proline, serine, threonine, tryptophan, | |
| | tyrosine and valine | |
| Non-Naturally- | Non-naturally occurring amino acids include, but are not limited to: | N/A |
| Occurring Amino Acids | ϵ-N Lysine, β-alanine, ornithine, norleucine, norvaline, | |
| | hydroxyproline, thyroxine, γ-amino butyric acid, homoserine, | |
| | citrulline, aminobenzoic acid, 6-aminocaproic acid (Aca; 6- | |
| | Aminohexanoic acid), hydroxyproline, mercaptopropionic acid | |
| | (MPA), 3-nitro-tyrosine, pyroglutamic acid, and the like | |
| Chemicals | -NHCH(X)CH₂SCH₂CO-, | N/A |
| | -NHCH(X)CH₂SCH₂CO(ϵN)Lys-, | |
| | -NHCH(X)CH₂S-succinimidyl(ϵN)Lys-, | |
| | -NHCH(X)CH₂S-(succinimidyl)- | |
| Gly-Gly | -GG- | N/A |
| Epsilon-N Lysine | ϵ-K | N/A |
| Epsilon-N Lysine-KKK | ϵ-K-KKK | 53 |
| KKK-Epsilon-N Lysine | KKK-ϵ-K | 54 |
| Hinge Sequence | Pro-Pro-Xaa-Pro-Xaa-Pro | 55 |
| CpG1 | 5′ TCg TCg TTT TgT CgT TTT gTC gTT TTg TCg TT 3′ | 146 |
| | (fully phosphorothioated) | |
| CpG2 | Phosphate TCg TCg TTT TgT CgT TTT gTC gTT 3′ | 147 |
| | (fully phosphorothloated) | |
| CpG3 | 5′ TCg TCg TTT TgT CgT TTT gTC gTT 3′ | 148 |
| | (fully phosphorothloated) | |

TABLE 3A
Examples of Target Antigenic Sites (B-Cell Epitopes)

| | | SEQ |
|---|---|---|
| | | ID |
| Description | Sequence* | NO |
| Aβ₁₋₁₄ | DAEFRHDSGYEVHH | 56 |
| Aβ₁₋₁₆ | DAEFRHDSGYEVHHQK | 57 |
| Aβ₁₇₋₄₂ | LVFFAEDVGSNKGAIIGLMVGGVVIA | 58 |
| Aβ₁₋₂₈ | DAEFRHDSGYEVHHQKLVFFAEDVGSNK | 59 |
| Aβ₁₋₄₂ | DAEFRHDSGYEVHHQKLVFFAEDVGSNKGAIIGLMVGGVVIA | 60 |
| α-Syn₁₁₁₋₁₃₂ | GILEDMPVDPDNEAYEMPSEEG | 61 |
| (Derived from GenBank: NP_000336) | | |
| IgE EMPD₁₋₃₉ | GLAGGSAQSQRAPDRVLCHSGQQQGLPRAAGGSVPHPRC | 62 |
| Tau₃₇₉₋₄₀₈ | RENAKAKTDHGAEIVYKSPVVSGDTSPRHL | 63 |
| (Derived from GenBank: AGF19246.1) | | |
| Tau₁₄₅₋₁₆₀ | ADGKTKIATPRGAAPP | 69 |
| Tau₁₋₅₀ | MAEPRQEFEVMEDHAGTYGLGDRKDQGGYTMHQDQEGDTDAGLKESPLQT | 70 |
| Tau₂₉₇₋₃₁₁ | IKHVPGGGSVQIVYK | 71 |
| IL-31₉₇₋₁₄₄ | LSDKNIIDKIIEQLDKLKFQHEPETEISVPADTFECKSFILTILQQFS | 64 |
| (Derived from Uniprot C7G0W1-1; | | |
| GenBank: BAH97742.1) | | |
| IL-31₈₅₋₁₁₅ | CPAIRAYLKTIRQLDNKSVIDEIIEHLDKLC | 72 |
| Melanoma Neoantigen | SGSPPLRVSVGDFSQEFSPQEAQQD | 73 |
| Histone 3 Variant H3.3K27M | RMSAPSTGGV | 74 |
| for Glioma | | |
| KRAS Metastatic Colorectal Cancer | MTEYKLVVVGA [symbol] GVGKSALTIQLI | 75 |
| (MUTANT G-to-D) | | |
| IL-6₇₃₋₈₃ | CFQSGFNEETC | 145 |
| IL-6₇₂₋₈₂ (rat counterpart) | CFQTGYNQEIC | 242 |

*The amino acids replaced by Cysteine are underlined.

TABLE 3B
Examples of Target Antigenic Sites (CTL Epitopes)

| Description | Sequence | SEQ ID NO |
|---|---|---|
| HIV CTL Gag P24 | SILDIKQGPKEPFRDYVDRFYKTLRAEQASQE | 76 |
| HIV CTL Gag P17 | ASRELERFAVNPGLLETSEGCR | 77 |
| HIV CTL Gag 293-312 | FRDYVDRFYKTLRAEQASQE | 78 |
| HIV CTL POL PRT | PRTKMIGGIGGFIKVRQYDQILIECGHKAIGTVLV | 79 |
| HIV CTL POL RT | LRWGFTTPDKKHQKEPPFLWMGYELH | 80 |
| HIV CTL POL INT | TKELQKQITKIQNFRVY | 81 |
| HIV CTL VIF | CFADSAIRKAILGHIV | 82 |
| HSV CTL VP11/12₆₆₋₇₄ | FLTCTDRSV | 83 |
| HSV CTL VP11/12₁₉₇₋₂₀₅ | RIQQYMFFM | 84 |
| HSV CTL VP11/12₂₂₀₋₂₂₈ | RLNELLAYV | 85 |
| HSV CTL VP11/12₂₃₀₋₂₄₆ | VLYRWASWMLWTTDKHV | 86 |
| HSV CTL VP11/12₇₀₂₋₇₁₀ | ALSALLTKL | 87 |
| HSV CTL γ1 gB₄₉₈ | SSIEFARL | 88 |
| HSV CTL γ1 gB₄₅₂ | YQPLLSNTL | 89 |
| HSV CTL γ1 gB₅₆₀ | SARMLGDVM | 90 |
| HSV CTL γ1 gG₄ | GAMRAVVPI | 91 |
| HSV CTL β PR1₉₈₂ | FAPLFTNL | 92 |
| HSV CTL β PR1₈₂₂ | QTFDFGRL | 93 |
| HSV CTL β PR1₃₇₂ | FGLLNYALV | 94 |
| HSV CTL β ICP₈₇₆ | GAINFINL | 95 |
| HSV CTL β ICP₁₆₈ | AVCINNTFLHL | 96 |
| HSV CTL β PR2₈₁ | SFYRFLFAFL | 97 |
| HSV CTL β PR2₂₇₉ | AAIENYVRF | 98 |
| HSV CTL β UL9₂₅₁ | FLPRLGTEL | 99 |
| HSV CTL β UL41₁₈₁ | LGYAYINS | 100 |
| HSV CTL γ2 gC₈ | LAVVLWSLL | 101 |
| HSV CTL β UL28₆₂₉ | YSVENVGLL | 102 |
| HSV CTL γ2 gH₃₉₁ | FAFVNAAHA | 103 |
| HSV CTL γ2 gK₅₄ | WMKMNQTLL | 104 |
| HSV CTL gB₁₆₁₋₁₈₀ | ATMYYKDVTVSQVWFGHRYS | 105 |
| HSV CTL gB₆₆₆₋₆₈₀ | TVSTFIDLNITMLED | 106 |
| FMDV CTL L₆₅ | EPFFDWVY | 107 |
| FMDV CTL VP4₂₂₇ | YMQQYQNSM | 108 |
| FMDV CTL VP2₃₁₄ | SSVGVTYGY | 109 |
| FMDV CTL VP2₃₄₆ | RFFKTHLF | 110 |
| FMDV CTL VP2₃₈₅ | AYMRNGWDVEV | 111 |
| FMDV CTL VP2₄₂₁ | RELYQLTLFPHQF | 112 |
| FMDV CTL VP3₆₂₂ | KARYMIAY | 113 |
| FMDV CTL 2C₁₃₀₉ | IIATTNLY | 114 |
| FMDV CTL 2C₁₃₆₉ | FQYDCALL(NGM) | 115 |
| FMDV CTL 3C₁₇₂₀ | MLSDAALMVL | 116 |
| FMDV CTL 3D₂₀₈₃ | WQRFGTHFAQYRNVWDVDY | 117 |
| FMDV CTL 3D₂₁₅₉ | NTILNNIYV(LY) | 118 |
| FMDV CTL 3D_{2231/43/58} | SITDVTFLKKKHMDYGTGFYKK KTLEAILSF | 119 |
| FMDV CTL 3D₂₂₉₅ | FEPFQGLFEIPSYRSLY | 120 |
| FMDV CTL 3D₂₃₀₂ | FEIPSYRSLY | 121 |
| FMDV CTL Type O ₇₅₀ | RRQHTDVSF | 122 |
| FMDV CTL Type O ₈₈₁ | RTLPTSFNY | 123 |
| PRRSV CTL ORF1a | RLGKIISLCQVIED | 124 |
| PRRSV CTL ORF1a | VPVITCGWHLLAII | 125 |
| PRRSV CTL ORF7 | LSDSGRISY | 126 |
| PRRSV CTL ORF2 | RTAPNEIAF | 127 |
| PRRSV CTL ORF2a | ASDWFAPRY | 128 |
| PRRSV CTL ORF5 | MSWRYSCTRY | 129 |
| PRRSV CTL NSP9 | TTMPSGFELY | 130 |
| PRRSV CTL NSP10 | NSFLDEAAY | 131 |
| PRRSV CTL ORF6 | RGRLLGLLHL | 132 |
| PRRSV CTL ORF5 | LYRWRSPVI | 133 |
| PRRSV CTL NSP9 | MPNYHWWVEH | 134 |
| PRRSV CTL NSP9 | EVALSAQII | 135 |
| PRRSV CTL Type 1₄₅₉₆ | CLFAILLAT | 136 |
| PRRSV CTL Type 1₄₇₂₁ | CAFAAFVCFVIR | 137 |
| PRRSV CTL Type 1₅₀₂₈ | KPEKPHFPL | 138 |
| PRRSV CTL Type 1₅₀₈₃ | FMLPVAHTV | 139 |
| PRRSV CTL Type 2₂₇₀₂ | TMPPGFELY | 140 |
| PRRSV CTL Type 2₄₇₆₅ | LAALICFVIRLAKNC | 141 |
| PRRSV CTL Type 2₄₇₉₇ | KGRLYRWRSPVII/VEK | 142 |
| CSFV CTL Type 1₁₄₄₆ | KHKVRNEVMVHWFDD | 143 |
| CSFV CTL Type 1₂₂₇₆ | ENALLVALF | 144 |

TABLE 4
Exemplary Peptide Immunogen Constructs

| Description | Sequence | SEQ ID NO |
|---|---|---|
| Aβ₁₋₁₄-ϵK-KKK-MVF4 Th | DAEFRHDSGYEVHH-ϵK-KKK-ISISEIKGVIVHKIETILF | 65 |
| | T RT TR | |
| Aβ₁₋₁₄-ϵK-HBsAg2 Th | DAEFRHDSGYEVHH-ϵK-KKK-IITITRIITIPQSLD | 66 |
| | FFLL L ITTI | |
| Aβ₁₋₁₄-ϵK-KKK-MVF5 Th | DAEFRHDSGYEVHH-ϵK-KKK-ISITEIKGVIVHRIETILF | 67 |
| Aβ₁₋₁₄-ϵK-HBsAg3 Th | DAEFRHDSGYEVHH-ϵK-KKK-IITITRIITIITTID | 68 |
| UBITh ®3-ϵK-KKK-rat IL-6 | ISISEIKGVIVHKIETILF-ϵK-KKK-CFQTGYNQEIC | 243 |
| | T RT TR | |

TABLE 5
Alpha-Synuclein constructs used in FIGS. 3, 4A, and 4B

| No. | Description | Sequence | SEQ ID NO |
|---|---|---|---|
| 01 | UBITh1-ϵK-KKK-α-Syn (G111-G132) | ISITEIKGVIVHRIETILF-ϵK-KKK-GILEDMPVDPDNEAYEMPSEEG | 149 |
| 02 | UBITh2-ϵK-KKK-α-Syn (G111-G132) | KKKIITITRIITIITTID-ϵK-KKK-GILEDMPVDPDNEAYEMPSEEG | 150 |
| 03 | UBITh3-ϵK-KKK-α-Syn (G111-G132) | ISISEIKGVIVHKIETILF-ϵK-KKK-GILEDMPVDPDNEAYEMPSEEG | 151 |
| | | T RT TR | |
| 04 | Clostridium tetani1 Th-ϵK-KKK-α-Syn (G111-G132) | KKQYIKANSKFIGITEL-ϵK-KKK-GILEDMPVDPDNEAYEMPSEEG | 152 |
| 05 | MvF1 Th-ϵK-KKK-α-Syn (G111-G132) | LSEIKGVIVHRLEGV-ϵK-KKK-GILEDMPVDPDNEAYEMPSEEG | 153 |
| 06 | Bordetella pertussis Th-ϵK-KKK-α-Syn (G111-G132) | GAYARCPNGTRALTVAELRGNAEL-ϵK-KKK-GILEDMPVDPDNEAYEMPSEEG | 154 |
| 07 | Clostridium tetani2 Th-ϵK-KKK-α-Syn (G111-G132) | WVRDIIDDFTNESSQKT-ϵK-KKK-GILEDMPVDPDNEAYEMPSEEG | 155 |
| 08 | Diphtheria Th-ϵK-KKK-α-Syn (G111-G132) | DSETADNLEKTVAALSILPGHGC-ϵK-KKK-GILEDMPVDPDNEAYEMPSEEG | 156 |
| 09 | Plasmodium falciparum Th-ϵK-KKK-α-Syn (G111-G132) | DHEKKHAKMEKASSVFNVVNS-ϵK-KKK-GILEDMPVDPDNEAYEMPSEEG | 157 |
| 10 | Schistosoma mansoni Th-ϵK-KKK-α-Syn (G111-G132) | KWFKTNAPNGVDEKHRH-ϵK-KKK-GILEDMPVDPDNEAYEMPSEEG | 158 |
| 11 | Cholera Toxin Th-ϵK-KKK-α-Syn (G111-G132) | ALNIWDRFDVFCTLGATTGYLKGNS-ϵK-KKK-GILEDMPVDPDNEAYEMPSEEG | 159 |
| 12 | MvF2 Th-ϵK-KKK-α-Syn (G111-G132) | ISEIKGVIVHKIEGI-ϵK-KKK-GILEDMPVDPDNEAYEMPSEEG | 160 |
| 13 | KKKMvF3 Th-ϵK-KKK-α-Syn (G111-G132) | KKKISISEIKGVIVHKIEGILF-ϵK-KKK-GILEDMPVDPDNEAYEMPSEEG | 161 |
| | | T RT TR T | |
| 14 | HBsAg1 Th-ϵK-KKK-α-Syn (G111-G132) | KKKLFLLTKLLTLPQSLD-ϵK-KKK-GILEDMPVDPDNEAYEMPSEEG | 162 |
| | | RRRIKII RII I L IR | |
| | | VRVV VV V I V | |
| | | F FF FF F V F | |
| | | F | |
| 15 | HBsAg2 Th-ϵK-KKK-α-Syn (G111-G132) | KKKIITITRIITIPQSLD-ϵK-KKK-GILEDMPVDPDNEAYEMPSEEG | 163 |
| | | FFLL L ITTI | |
| 16 | Influenza MP1_1 Th-ϵK-KKK-α-Syn (G111-G132) | FVFTLTVPSER-ϵK-KKK-GILEDMPVDPDNEAYEMPSEEG | 164 |
| 17 | Influenza MP1_2 Th-ϵK-KKK-α-Syn (G111-G132) | SGPLKAEIAQRLEDV-ϵK-KKK-GILEDMPVDPDNEAYEMPSEEG | 165 |
| 18 | Influenza NSP1 Th-ϵK-KKK-α-Syn (G111-G132) | DRLRRDQKS-ϵK-KKK-GILEDMPVDPDNEAYEMPSEEG | 166 |
| 19 | EBV BHRF1 Th-ϵK-KKK-α-Syn (G111-G132) | AGLTLSLLVICSYLFISRG-ϵK-KKK-GILEDMPVDPDNEAYEMPSEEG | 167 |
| 20 | Clostridium tetani TT1 Th-ϵK-KKK-α-Syn (G111-G132) | QYIKANSKFIGITEL-ϵK-KKK-GILEDMPVDPDNEAYEMPSEEG | 168 |
| 21 | EBV EBNA-1 Th-ϵK-KKK-α-Syn (G111-G132) | PGPLRESIVCYFMVFLQTHI-ϵK-KKK-GILEDMPVDPDNEAYEMPSEEG | 169 |
| 22 | Clostridium tetani TT2 Th-ϵK-KKK-α-Syn (G111-G132) | FNNFTVSFWLRVPKVSASHLE-ϵK-KKK-GILEDMPVDPDNEAYEMPSEEG | 170 |
| 23 | Clostridium tetani TT3 Th-ϵK-KKK-α-Syn (G111-G132) | KFIIKRYTPNNEIDSF-ϵK-KKK-GILEDMPVDPDNEAYEMPSEEG | 171 |
| 24 | Clostridium tetani TT4 Th-ϵK-KKK-α-Syn (G111-G132) | VSIDKFRIFCKALNPK-ϵK-KKK-GILEDMPVDPDNEAYEMPSEEG | 172 |
| 25 | EBV CP Th-ϵK-KKK-α-Syn (G111-G132) | VPGLYSPCRAFFNKEELL-ϵK-KKK-GILEDMPVDPDNEAYEMPSEEG | 173 |
| 26 | HCMV IE1 Th-ϵK-KKK-α-Syn (G111-G132) | DKREMWMACIKELH-ϵK-KKK-GILEDMPVDPDNEAYEMPSEEG | 174 |
| 27 | EBV GP340 Th-ϵK-KKK-α-Syn (G111-G132) | TGHGARTSTEPTTDY-ϵK-KKK-GILEDMPVDPDNEAYEMPSEEG | 175 |
| 28 | EBV BPLF1 Th-ϵK-KKK-α-Syn (G111-G132) | KELKRQYEKKLRQ-ϵK-KKK-GILEDMPVDPDNEAYEMPSEEG | 176 |
| 29 | EBV EBNA-2 Th-ϵK-KKK-α-Syn (G111-G132) | TVFYNIPPMPL-ϵK-KKK-GILEDMPVDPDNEAYEMPSEEG | 177 |

TABLE 6
IgE-EMPD constructs used in FIG. 5

| No. | Description | Sequence | SEQ ID NO |
|---|---|---|---|
| 01 | IgE-EMPD (G1-C39)-ϵK-UBITh1 | GLAGGSAQSQRAPDRVLCHSGQQQGLPRAAGGSVPHPRC-ϵK-ISITEIKGVIVHRIETILF | 178 |
| 02 | UBITh2-ϵK-IgE-EMPD (G1-C39) | KKKIITITRIITIITTID-ϵK-GLAGGSAQSQRAPDRVLCHSGQQQGLPRAAGGSVPHPRC | 179 |
| 03 | UBITh3-ϵK-IgE-EMPD (G1-C39) | ISISEIKGVIVHKIETILF-ϵK-GLAGGSAQSQRAPDRVLCHSGQQQGLPRAAGGSVPHPRC | 180 |
| | | T RT TR | |
| 04 | Clostridium tetani1 Th-ϵK-IgE-EMPD (G1-C39) | KKQYIKANSKFIGITEL-ϵK-GLAGGSAQSQRAPDRVLCHSGQQQGLPRAAGGSVPHPRC | 181 |
| 05 | MvF1 Th-ϵK-IgE-EMPD (G1-C39) | LSEIKGVIVHRLEGV-ϵK-GLAGGSAQSQRAPDRVLCHSGQQQGLPRAAGGSVPHPRC | 182 |
| 06 | Bordetella pertussis Th-ϵK-IgE-EMPD (G1-C39) | GAYARCPNGTRALTVAELRGNAEL-ϵK-GLAGGSAQSQRAPDRVLCHSGQQQGLPRAAGGSVPHPRC | 183 |
| 07 | Clostridium tetani2 Th-ϵK-IgE-EMPD (G1-C39) | WVRDIIDDFTNESSQKT-ϵK-GLAGGSAQSQRAPDRVLCHSGQQQGLPRAAGGSVPHPRC | 184 |
| 08 | Diphtheria Th-ϵK-IgE-EMPD (G1-C39) | DSETADNLEKTVAALSILPGHGC-ϵK-GLAGGSAQSQRAPDRVLCHSGQQQGLPRAAGGSVPHPRC | 185 |
| 09 | Plasmodium falciparum Th-ϵK-IgE-EMPD (G1-C39) | DHEKKHAKMEKASSVFNVVNS-ϵK-GLAGGSAQSQRAPDRVLCHSGQQQGLPRAAGGSVPHPRC | 186 |
| 10 | Schistosoma mansoni Th-ϵK-IgE-EMPD (G1-C39) | KWFKTNAPNGVDEKHRH-ϵK-GLAGGSAQSQRAPDRVLCHSGQQQGLPRAAGGSVPHPRC | 187 |
| 11 | Cholera Toxin Th-ϵK-IgE-EMPD (G1-C39) | ALNIWDRFDVFCTLGATTGYLKGNS-ϵK-GLAGGSAQSQRAPDRVLCHSGQQQGLPRAAGGSVPHPRC | 188 |
| 12 | MvF2 Th-ϵK-IgE-EMPD (G1-C39) | ISEIKGVIVHKIEGI-ϵK-GLAGGSAQSQRAPDRVLCHSGQQQGLPRAAGGSVPHPRC | 189 |
| 13 | KKKMvF3 Th-ϵK-IgE-EMPD (G1-C39) | KKKISISEIKGVIVHKIEGILF-ϵK-GLAGGSAQSQRAPDRVLCHSGQQQGLPRAAGGSVPHPRC | 190 |
| | | T RT TR T | |
| 14 | HBsAg1 Th-ϵK-IgE-EMPD (G1-C39) | KKKLFLLTKLLTLPQSLD-ϵK-GLAGGSAQSQRAPDRVLCHSGQQQGLPRAAGGSVPHPRC | 191 |
| | | RRRIKII RII I L IR | |
| | | VRVV VV V I V | |
| | | F FF FF F V F | |
| | | F | |
| 15 | HBsAg2 Th-ϵK-IgE-EMPD (G1-C39) | KKKIITITRIITIPQSLD-ϵK-GLAGGSAQSQRAPDRVLCHSGQQQGLPRAAGGSVPHPRC | 192 |
| | | FFLL L ITTI | |
| 16 | Influenza MP1_1 Th-ϵK-IgE-EMPD (G1-C39) | FVFTLTVPSER-ϵK-GLAGGSAQSQRAPDRVLCHSGQQQGLPRAAGGSVPHPRC | 193 |
| 17 | Influenza MP1_2 Th-ϵK-IgE-EMPD (G1-C39) | SGPLKAEIAQRLEDV-ϵK-GLAGGSAQSQRAPDRVLCHSGQQQGLPRAAGGSVPHPRC | 194 |
| 18 | Influenza NSP1 Th-ϵK-IgE-EMPD (G1-C39) | DRLRRDQKS-ϵK-GLAGGSAQSQRAPDRVLCHSGQQQGLPRAAGGSVPHPRC | 195 |
| 19 | EBV BHRF1 Th-ϵK-IgE-EMPD (G1-C39) | AGLTLSLLVICSYLFISRG-ϵK-GLAGGSAQSQRAPDRVLCHSGQQQGLPRAAGGSVPHPRC | 196 |
| 20 | Clostridium tetani TT1 Th-ϵK-IgE-EMPD (G1-C39) | QYIKANSKFIGITEL-ϵK-GLAGGSAQSQRAPDRVLCHSGQQQGLPRAAGGSVPHPRC | 197 |
| 21 | EBV EBNA-1 Th-ϵK-IgE-EMPD (G1-C39) | PGPLRESIVCYFMVFLQTHI-ϵK-GLAGGSAQSQRAPDRVLCHSGQQQGLPRAAGGSVPHPRC | 198 |
| 22 | Clostridium tetani TT2 Th-ϵK-IgE-EMPD (G1-C39) | FNNFTVSFWLRVPKVSASHLE-ϵK-GLAGGSAQSQRAPDRVLCHSGQQQGLPRAAGGSVPHPRC | 199 |
| 23 | Clostridium tetani TT3 Th-ϵK-IgE-EMPD (G1-C39) | KFIIKRYTPNNEIDSF-ϵK-GLAGGSAQSQRAPDRVLCHSGQQQGLPRAAGGSVPHPRC | 200 |
| 24 | Clostridium tetani TT4 Th-ϵK-IgE-EMPD (G1-C39) | VSIDKFRIFCKALNPK-ϵK-GLAGGSAQSQRAPDRVLCHSGQQQGLPRAAGGSVPHPRC | 201 |
| 25 | EBV CP Th-ϵK-IgE-EMPD (G1-C39) | VPGLYSPCRAFFNKEELL-ϵK-GLAGGSAQSQRAPDRVLCHSGQQQGLPRAAGGSVPHPRC | 202 |
| 26 | HCMV IE1 Th-ϵK-IgE-EMPD (G1-C39) | DKREMWMACIKELH-ϵK-GLAGGSAQSQRAPDRVLCHSGQQQGLPRAAGGSVPHPRC | 203 |
| 27 | EBV GP340 Th-ϵK-IgE-EMPD (G1-C39) | TGHGARTSTEPTTDY-ϵK-GLAGGSAQSQRAPDRVLCHSGQQQGLPRAAGGSVPHPRC | 204 |
| 28 | EBV BPLF1 Th-ϵK-IgE-EMPD (G1-C39) | KELKRQYEKKLRQ-ϵK-GLAGGSAQSQRAPDRVLCHSGQQQGLPRAAGGSVPHPRC | 205 |
| 29 | EBV EBNA-2 Th-ϵK-IgE-EMPD (G1-C39) | TVFYNIPPMPL-ϵK-GLAGGSAQSQRAPDRVLCHSGQQQGLPRAAGGSVPHPRC | 206 |

TABLE 7
IL-6 constructs used in FIGS. 3 and 6

| | | | SEQ |
|---|---|---|---|
| | | | ID |
| No. | Description | Sequence | NO |
| 01 | UBITh1-ϵK-KKK-IL6 (C73-C83) | ISITEIKGVIVHRIETILF-ϵK-KKK-CFQSGFNEETC | 207 |
| 02 | UBITh2-ϵK-KKK-IL6 (C73-C83) | KKKIITITRIITIITTID-ϵK-KKK-CFQSGFNEETC | 208 |
| 03 | UBITh3-ϵK-KKK-IL6 (C73-C83) | ISISEIKGVIVHKIETILF-ϵK-KKK-CFQSGFNEETC | 209 |
| | | T RT TR | |
| 04 | Clostridium tetani1 Th-KKK-ϵK-IL6 (C73-C83) | KKQYIKANSKFIGITEL-KKK-ϵK-CFQSGFNEETC | 210 |
| 05 | MvF1 Th-KKK-ϵK-IL6 (C73-C83) | LSEIKGVIVHRLEGV-KKK-ϵK-CFQSGFNEETC | 211 |
| 06 | Bordetella pertussis Th-KKK-ϵK-IL6 (C73-C83) | GAYARCPNGTRALTVAELRGNAEL-KKK-ϵK-CFQSGFNEETC | 212 |
| 07 | Clostridium tetani2 Th-KKK-ϵK-IL6 (C73-C83) | WVRDIIDDFTNESSQKT-KKK-ϵK-CFQSGFNEETC | 213 |
| 08 | Diphtheria Th-KKK-ϵK-IL6 (C73-C83) | DSETADNLEKTVAALSILPGHGC-KKK-ϵK-CFQSGFNEETC | 214 |
| 09 | Plasmodium falciparum Th-KKK-ϵK-IL6 (C73-C83) | DHEKKHAKMEKASSVFNVVNS-KKK-ϵK-CFQSGFNEETC | 215 |
| 10 | Schistosoma mansoni Th-KKK-ϵK-IL6 (C73-C83) | KWFKTNAPNGVDEKHRH-KKK-ϵK-CFQSGFNEETC | 216 |
| 11 | Cholera Toxin Th-KKK-ϵK-IL6 (C73-C83) | ALNIWDRFDVFCTLGATTGYLKGNS-KKK-ϵK-CFQSGFNEETC | 217 |
| 12 | MvF2 Th-KKK-ϵK-IL6 (C73-C83) | ISEIKGVIVHKIEGI-KKK-ϵK-CFQSGFNEETC | 218 |
| 13 | KKKMvF3 Th-KKK-ϵK-IL6 (C73-C83) | KKKISISEIKGVIVHKIEGILF-KKK-ϵK-CFQSGFNEETC | 219 |
| | | T RT TR T | |
| 14 | HBsAg1 Th-KKK-ϵK-IL6 (C73-C83) | KKKLFLLTKLLTLPQSLD-KKK-ϵK-CFQSGFNEETC | 220 |
| | | RRRIKII RII I L IR | |
| | | VRVV VV V I V | |
| | | F FF FF F V F | |
| | | F | |
| 15 | HBsAg2 Th-KKK-ϵK-IL6 (C73-C83) | KKKIITITRIITIPQSLD-KKK-ϵK-CFQSGFNEETC | 221 |
| | | FFLL L ITTI | |
| 16 | Influenza MP1_1 Th-KKK-ϵK-IL6 (C73-C83) | FVFTLTVPSER-KKK-ϵK-CFQSGFNEETC | 222 |
| 17 | Influenza MP1_2 Th-KKK-ϵK-IL6 (C73-C83) | SGPLKAEIAQRLEDV-KKK-ϵK-CFQSGFNEETC | 223 |
| 18 | Influenza NSP1 Th-KKK-ϵK-IL6 (C73-C83) | DRLRRDQKS-KKK-ϵK-CFQSGFNEETC | 224 |
| 19 | EBV BHRF1 Th-KKK-ϵK-IL6 (C73-C83) | AGLTLSLLVICSYLFISRG-KKK-ϵK-CFQSGFNEETC | 225 |
| 20 | Clostridium tetani TT1 Th-KKK-ϵK-IL6 (C73-C83) | QYIKANSKFIGITEL-KKK-ϵK-CFQSGFNEETC | 226 |
| 21 | EBV EBNA-1 Th-KKK-ϵK-IL6 (C73-C83) | PGPLRESIVCYFMVFLQTHI-KKK-ϵK-CFQSGFNEETC | 227 |
| 22 | Clostridium tetani TT2 Th-KKK-ϵK-IL6 (C73-C83) | FNNFTVSFWLRVPKVSASHLE-KKK-ϵK-CFQSGFNEETC | 228 |
| 23 | Clostridium tetani TT3 Th-KKK-ϵK-IL6 (C73-C83) | KFIIKRYTPNNEIDSF-KKK-ϵK-CFQSGFNEETC | 229 |
| 24 | Clostridium tetani TT4 Th-KKK-ϵK-IL6 (C73-C83) | VSIDKFRIFCKALNPK-KKK-ϵK-CFQSGFNEETC | 230 |
| 25 | EBV CP Th-KKK-ϵK-IL6 (C73-C83) | VPGLYSPCRAFFNKEELL-KKK-ϵK-CFQSGFNEETC | 231 |
| 26 | HCMV IE1 Th-KKK-ϵK-IL6 (C73-C83) | DKREMWMACIKELH-KKK-ϵK-CFQSGFNEETC | 232 |
| 27 | EBV GP340 Th-KKK-ϵK-IL6 (C73-C83) | TGHGARTSTEPTTDY-KKK-ϵK-CFQSGFNEETC | 233 |
| 28 | EBV BPLF1 Th-KKK-ϵK-IL6 (C73-C83) | KELKRQYEKKLRQ-KKK-ϵK-CFQSGFNEETC | 234 |
| 29 | EBV EBNA-2 Th-KKK-ϵK-IL6 (C73-C83) | TVFYNIPPMPL-KKK-ϵK-CFQSGFNEETC | 235 |

TABLE 8
Mixtures of peptide immunogens administered to guinea pigs
to evaluate immunogenicities of 29 different Th epitopes

| No. | Description | SEQ ID NO |
|---|---|---|
| 01 | UBITh1-εK-KKK-α-Syn (G111-G132) | 149 |
| | IgE-EMPD (G1-C39)-εK-UBITh1 | 178 |
| | UBITh1-εK-KKK-IL6 (C73-C83) | 207 |
| 02 | UBITh2-εK-KKK-α-Syn (G111-G132) | 150 |
| | UBITh2-εK-IgE-EMPD (G1-C39) | 179 |
| | UBITh2-εK-KKK-IL6 (C73-C83) | 208 |
| 03 | UBITh3-εK-KKK-α-Syn (G111-G132) | 151 |
| | UBITh3-εK-IgE-EMPD (G1-C39) | 180 |
| | UBITh3-εK-KKK-IL6 (C73-C83) | 209 |
| 04 | Clostridium tetani1 Th-εK-KKK-α-Syn (G111-G132) | 152 |
| | Clostridium tetani1 Th-εK-IgE-EMPD (G1-C39) | 181 |
| | Clostridium tetani1 Th-KKK-εK-IL6 (C73-C83) | 210 |
| 05 | MvFl Th-εK-KKK-α-Syn (G111-G132) | 153 |
| | MvFl Th-εK-IgE-EMPD (G1-C39) | 182 |
| | MvFl Th-KKK-εK-IL6 (C73-C83) | 211 |
| 06 | Bordetella pertussis Th-εK-KKK-α-Syn (G111-G132) | 154 |
| | Bordetella pertussis Th-εK-IgE-EMPD (G1-C39) | 183 |
| | Bordetella pertussis Th-KKK-εK-IL6 (C73-C83) | 212 |
| 07 | Clostridium tetani2 Th-εK-KKK-α-Syn (G111-G132) | 155 |
| | Clostridium tetani2 Th-εK-IgE-EMPD (G1-C39) | 184 |
| | Clostridium tetani2 Th-KKK-εK-IL6 (C73-C83) | 213 |
| 08 | Diphtheria Th-εK-KKK-α-Syn (G111-G132) | 156 |
| | Diphtheria Th-εK-IgE-EMPD (G1-C39) | 185 |
| | Diphtheria Th-KKK-εK-IL6 (C73-C83) | 214 |
| 09 | Plasmodium falciparum Th-εK-KKK-α-Syn (G111-G132) | 157 |
| | Plasmodium falciparum Th-εK-IgE-EMPD (G1-C39) | 186 |
| | Plasmodium falciparum Th-KKK-εK-IL6 (C73-C83) | 215 |
| 10 | Schistosoma mansoni Th-εK-KKK-α-Syn (G111-G132) | 158 |
| | Schistosoma mansoni Th-εK-IgE-EMPD (G1-C39) | 187 |
| | Schistosoma mansoni Th-KKK-εK-IL6 (C73-C83) | 216 |
| 11 | Cholera Toxin Th-εK-KKK-α-Syn (G111-G132) | 159 |
| | Cholera Toxin Th-εK-IgE-EMPD (G1-C39) | 188 |
| | Cholera Toxin Th-KKK-εK-IL6 (C73-C83) | 217 |
| 12 | MvF2 Th-εK-KKK-α-Syn (G111-G132) | 160 |
| | MvF2 Th-εK-IgE-EMPD (G1-C39) | 189 |
| | MvF2 Th-KKK-εK-IL6 (C73-C83) | 218 |
| 13 | KKKMVF3 Th-εK-KKK-α-Syn (G111-G132) | 161 |
| | KKKMVF3 Th-εK-IgE-EMPD (G1-C39) | 190 |
| | KKKMVF3 Th-KKK-εK-IL6 (C73-C83) | 219 |
| 14 | HBsAg1 Th-εK-KKK-α-Syn (G111-G132) | 162 |
| | HBsAg1 Th-εK-IgE-EMPD (G1-C39) | 191 |
| | HBsAg1 Th-KKK-εK-IL6 (C73-C83) | 220 |
| 15 | HBsAg2 Th-εK-KKK-α-Syn (G111-G132) | 163 |
| | HBsAg2 Th-εK-IgE-EMPD (G1-C39) | 192 |
| | HBsAg2 Th-KKK-εK-IL6 (C73-C83) | 221 |
| 16 | Influenza MP1_1 Th-εK-KKK-α-Syn (G111-G132) | 164 |
| | Influenza MP1_1 Th-εK-IgE-EMPD (G1-C39) | 193 |
| | Influenza MP1_1 Th-KKK-εK-IL6 (C73-C83) | 222 |
| 17 | Influenza MP1_2 Th-εK-KKK-α-Syn (G111-G132) | 165 |
| | Influenza MP1_2 Th-εK-IgE-EMPD (G1-C39) | 194 |
| | Influenza MP1_2 Th-KKK-εK-IL6 (C73-C83) | 223 |
| 18 | Influenza NSP1 Th-εK-KKK-α-Syn (G111-G132) | 166 |
| | Influenza NSP1 Th-εK-IgE-EMPD (G1-C39) | 195 |
| | Influenza NSP1 Th-KKK-εK-IL6 (C73-C83) | 224 |
| 19 | EBV BHRF1 Th-εK-KKK-α-Syn (G111-G132) | 167 |
| | EBV BHRF1 Th-εK-IgE-EMPD (G1-C39) | 196 |
| | EBV BHRF1 Th-KKK-εK-IL6 (C73-C83) | 225 |
| 20 | Clostridium tetani TT1 Th-εK-KKK-α-Syn (G111-G132) | 168 |
| | Clostridium tetani TT1 Th-εK-IgE-EMPD (G1-C39) | 197 |
| | Clostridium tetani TT1 Th-KKK-εK-IL6 (C73-C83) | 226 |
| 21 | EBV EBNA-1 Th-εK-KKK-α-Syn (G111-G132) | 169 |
| | EBV EBNA-1 Th-εK-IgE-EMPD (G1-C39) | 198 |
| | EBV EBNA-1 Th-KKK-εK-IL6 (C73-C83) | 227 |
| 22 | Clostridium tetani TT2 Th-εK-KKK-α-Syn (G111-G132) | 170 |
| | Clostridium tetani TT2 Th-εK-IgE-EMPD (G1-C39) | 199 |
| | Clostridium tetani TT2 Th-KKK-εK-IL6 (C73-C83) | 228 |
| 23 | Clostridium tetani TT3 Th-εK-KKK-α-Syn (G111-G132) | 171 |
| | Clostridium tetani TT3 Th-εK-IgE-EMPD (G1-C39) | 200 |
| | Clostridium tetani TT3 Th-KKK-εK-IL6 (C73-C83) | 229 |
| 24 | Clostridium tetani TT4 Th-εK-KKK-α-Syn (G111-G132) | 172 |
| | Clostridium tetani TT4 Th-εK-IgE-EMPD (G1-C39) | 201 |
| | Clostridium tetani TT4 Th-KKK-εK-IL6 (C73-C83) | 230 |
| 25 | EBV CP Th-εK-KKK-α-Syn (G111-G132) | 173 |
| | EBV CP Th-εK-IgE-EMPD (G1-C39) | 202 |
| | EBV CP Th-KKK-εK-IL6 (C73-C83) | 231 |
| 26 | HCMV IE1 Th-εK-KKK-α-Syn (G111-G132) | 174 |
| | HCMV IE1 Th-εK-IgE-EMPD (G1-C39) | 203 |
| | HCMV IE1 Th-KKK-εK-IL6 (C73-C83) | 232 |
| 27 | EBV GP340 Th-εK-KKK-α-Syn (G111-G132) | 175 |
| | EBV GP340 Th-εK-IgE-EMPD (G1-C39) | 204 |
| | EBV GP340 Th-KKK-εK-IL6 (C73-C83) | 233 |
| 28 | EBV BPLF1 Th-εK-KKK-α-Syn (G111-G132) | 176 |
| | EBV BPLF1 Th-εK-IgE-EMPD (G1-C39) | 205 |
| | EBV BPLF1 Th-KKK-εK-IL6 (C73-C83) | 234 |
| 29 | EBV EBNA-2 Th-εK-KKK-α-Syn (G111-G132) | 177 |
| | EBV EBNA-2 Th-εK-IgE-EMPD (G1-C39) | 206 |
| | EBV EBNA-2 Th-KKK-εK-IL6 (C73-C83) | 235 |

TABLE 9
Dipeptide Repeat (DPR) constructs used in FIG. 7

| Description | Sequence | SEQ ID NO |
|---|---|---|
| Short B Cell Epitope | GAGAGAGAGAGAGAGAGAGA-KKK-ϵK-ISITEIKGVIVHRIETILF | 236 |
| Medium B Cell Epitope | GAGAGAGAGAGAGAGAGAGAGAGAGAGAGA-KKK-ϵK-ISITEIKGVIVHRIETILF | 237 |
| Long B Cell Epitope | GAGAGAGAGAGAGAGAGAGAGAGAGAGAGAGAGAGAGAGAGAGAGAGAGA-KKK-ϵK- | 238 |
| | ISITEIKGVIVHRIETILF | |

TABLE 10
LHRH constructs used in FIGS. 13 and 14

| Description | Sequence | SEQ ID NO |
|---|---|---|
| TT1 Th-LHRH | KKQYIKANSKFIGITEL-EHWSYGLRPG | 239 |
| Invasin-MVF Th-LHRH | TAKSKKFPSYTATYQF-GG-LSEIKGVIVHRLEGV-GG-EHWSYGLRPG | 240 |
| Invasin-HBsAg Th-LHRH | TAKSKKFPSYTATYQF-GG-FFLLTRILTIPQSLE-GG-EHWSYGLRPG | 241 |

TABLE 11
Exclusive immunogenicity of Aβ₁₋₁₄ immunogens in guinea
pigs that target Aβ peptides but not Th epitopes

| | | Antibody titer at Week 8 (log₁₀) | | |
|---|---|---|---|---|
| | | Aβ₁₋₄₂ | MvF5 Th | HBsAg3 Th |
| | Animal | (SEQ ID | (SEQ ID | (SEQ ID |
| Peptide Immunogens | no. | NO: 60) | NO: 17) | NO: 28) |
| Aβ₁₋₁₄-εKKK-MvF5 Th | 1 | 4.68 | 0.21 | 0.31 |
| (SEQ ID NO: 67) + | 2 | 3.88 | 0.33 | 0.42 |
| Aβ₁₋₁₄-εK-HBsAg3 Th | 3 | 3.92 | 0.43 | 0.31 |
| (SEQ ID NO: 68) | 4 | 3.58 | 0.54 | 0.55 |
| | 5 | 3.35 | 0.52 | 0.38 |
| | 6 | 3.48 | 0.40 | 0.42 |
| | Mean | 3.82 | 0.41 | 0.39 |
| | (SD) | (0.48) | (0.12) | (0.09) |

TABLE 12
Measurement of cytokine concentration in UB311
vaccinated or normal Cynomolgus Macaque Peripheral Blood
Mononuclear Cells (PBMCs) collected at 15, 21 and 25.5 wpi
upon stimulation with Aβ₁₋₁₄, Aβ₁₋₄₂
peptides or PHA (Phytohemagglutin) mitogen

| | | Cytokine Concentration a (μg/mL) | | |
|---|---|---|---|---|
| | Vaccine | Aβ₁₋₁₄ | Aβ₁₋₄₂ | |
| Cytokine | dose | (SEQ ID NO: 56) | (SEQ ID NO: 58) | PHA |
| IL-2 | Placebo | BDL^{b} | 23.3 ± 13.1 | 90.6 ± 12.4 |
| | 150 μg | BDL | 19.4 ± 9.7 | 96.1 ± 13.3 |
| | 750 μg | BDL | 25.2 ± 11.8 | 97.5 ± 6.6 |
| IL-6 | Placebo | BDL | 23.1 ± 11.7 | 69.1 ± 12.0 |
| | 150 μg | BDL | 15.0 ± 9.1 | 70.6 ± 15.7 |
| | 750 μg | BDL | 23.4 ± 10.5 | 66.2 ± 7.3 |
| TNF-α | Placebo | BDL | 9.2 ± 5.3 | 91.0 ± 29.1 |
| | 150 μg | BDL | 7.9 ± 4.8 | 96.1 ± 22.2 |
| | 750 μg | BDL | 7.8 ± 5.9 | 89.0 ± 13.7 |

a Result was shown as mean ± standard deviation
^{b}BDL, below detection level

TABLE 13
Stimulation Index of PBMCs evaluated from
the 19 patients with Alzheimer's disease

| | Week 0 | Week 16 | Difference | Paired t-test |
|---|---|---|---|---|
| Peptide | Mean (SD) | Mean (SD) | Mean (SD) | p value |
| Aβ₁₋₁₄ | 0.93 (0.36) | 0.90 (0.22) | −0.03 (0.39) | 0.73 |
| (SEQ ID NO: 56) | | | | |
| Aβ₁₋₁₆ | 0.92 (0.30) | 0.98 (0.25) | 0.06 (0.40) | 0.54 |
| (SEQ ID NO: 57) | | | | |
| Aβ₁₋₂₈ | 0.96 (0.30) | 1.04 (0.34) | 0.08 (0.56) | 0.55 |
| (SEQ ID NO: 59) | | | | |
| Aβ₁₇₋₄₂ | 0.96 (0.34) | 1.04 (0.29) | 0.08 (0.49) | 0.47 |
| (SEQ ID NO: 58) | | | | |
| Aβ₁₋₄₂ | 0.97 (0.38) | 1.08 (0.49) | 0.10 (0.53) | 0.40 |
| (SEQ ID NO: 60) | | | | |
| p1412 | 0.87 (0.22) | 0.99 (0.33) | 0.11 (0.34) | 0.18 |
| (non-relevant | | | | |
| peptide) | | | | |
| PHA | 28.73 (14.2) | 27.75 (32.9) | −0.98 (26.6) | 0.87 |

TABLE 14
Cytokine concentrations in peripheral blood mononuclear cells (PBMC) evaluated from the 19
patients upon stimulation with Aβ peptides or PHA mitogen¹

| | Th1 | | | | Th 2 | | | | Both | |
|---|---|---|---|---|---|---|---|---|---|---|
| | IL2 | | IFN-γ | | IL-6 | | IL-10 | | TNF-α | |
| Peptide | W0 | W16 | W0 | W16 | W0 | W16 | W0 | W16 | W0 | W16 |
| Aβ₁₋₁₄ | 31.1 | 31.2 | 13.5 | 16.1 | 31.3 | 50.7 | 5.7 | 5.6 | 36.8 | 39.8 |
| (SEQ ID NO: 56) | (32.5) | (24.3) | (16.9) | (12.9) | (29.7) | (52.0) | (1.6) | (1.6) | (62.8) | (51.7) |
| Aβ₁₋₁₆ | 31.4 | 36.0 | 15.0 | 13.8 | 52.5 | 50.4 | 5.7 | 5.8 | 47.4 | 47.23 |
| (SEQ ID NO: 57) | (31.4) | (23.9) | (16.1) | (14.2) | (31.7) | (42.6) | (1.6) | (1.8) | (72.2) | (69.7) |
| Aβ₁₋₂₈ | 36.7 | 40.6 | 16.0 | 20.7 | 31.7 | 42.3 | 5.6 | 6.2 | 41.6 | 51.2 |
| (SEQ ID NO: 59) | (34.3) | (28.0) | (23.6) | (24.4) | (25.4) | (41.8) | (1.5) | (2.5) | (66.5) | (67.8) |
| Aβ₁₇₋₄₂ | 24.6 | 29.2 | 9.7 | 13.6 | >44.6 | 46.9 | 5.3 | 5.6 | 15.6 | 24.8 |
| (SEQ ID NO: 58) | (25.7) | (21.2) | (9.7) | (15.6) | (70.9)³ | (51.3) | (0.86) | (1.5) | (18.4) | (39.3) |
| Aβ₁₋₄₂ | 23.1 | 27.3 | 13.4 | >44.8 | >141 | >202 | 11.1 | 31.9 | >31.6 | >88.8 |
| (SEQ ID NO: 60) | (17.7) | (16.9) | (16.1) | (77.3) | (130)⁴ | (121)⁵ | (22.7) | (50.2) | (71.5)³ | (133)⁶ |
| p1412 | 30.9 | 40.0 | 14.4 | 21.7 | 31.8 | 60.7 | 5.3 | 5.2 | 17.1 | 20.9 |
| | (27.4) | (26.0) | (18.4) | (30.0) | (52.1) | (95.8) | (0.64) | (0.53) | (23.5) | (29.3) |
| PHA | 10.4⁷ | 12.8⁷ | >320 | >319 | >320 | >320 | 174 | >163 | >313 | >301 |
| | (11.3) | (6.5) | (0.00)² | (4.8)² | (0.00)² | (0.00)² | (84.8) | (99.7) | (30.5)² | (46.5)² |
| Cell control | 33.4 | 38.8 | 13.8 | 17.8 | 45.9 | 65.3 | 5.9 | 5.7 | 44.3 | 46.7 |
| | (24.9) | (33.1) | (12.3) | (18.2) | (41.9) | (76.5) | (2.5) | (1.6) | (70.9) | (67.8) |

¹Quantifiable range of the assay is between 5 and 320 pg/mL
²Concentration of >90% subjects were above the upper quantification limit (AQL > 320 pg/mL)
³One patient had an AQL value
⁴Six patients had AQL values
⁵Eight patients had AQL values
⁶Four patients had AQL values
⁷The lack of IL-2 production observed in response to PHA mitogen was consistent with data reported under similar experimental conditions

TABLE 15
Immunogenicity Enhancement of IL-6 B Cell Epitope Peptide (C73-C83) (SEQ ID NO: 145)
with Ranking Heterologous Th Epitope Peptides from Pathogenic Proteins

| Th | | | Recombinant | | | Th | | | Recombinant | | |
|---|---|---|---|---|---|---|---|---|---|---|---|
| epitope | | | human IL-6 | | | epitope | | | human IL-6 | | |
| SEQ ID | IL-6 peptide immunogen | Animal | ELISA Log₁₀ Titer | | | SEQ ID | IL-6 peptide immunogen | Animal | ELISA Log₁₀ Titer | | |
| NO | construct | ID | 0 wpi | 6 wpi | 8 wpi | epitope | construct | ID | 0 wpi | 6 wpi | 8 wpi |
| 17 | UBITh ®1-εK-KKK- | 6381 | 0.142 | 5.360 | 5.367 | 36 | Clostridium tetani TT2 Th- | 6438 | 0.084 | 4.796 | 4.936 |
| | IL-6 (C73-C83) | 6382 | 0.091 | 7.456 | 9.026 | | KKK-εK-IL-6 (C73-C83) | 6439 | 0.091 | 4.120 | 3.696 |
| | | 6383 | 0.098 | 5.459 | 6.674 | | | 6440 | 0.074 | 3.163 | 2.925 |
| | | Avg | 0.110 | 6.092 | 7.022 | | | Avg | 0.083 | 4.026 | 3.852 |
| 34 | Clostridium tetani TT1 Th- | 6432 | 0.064 | 5.135 | NS | 35 | Clostridium tetani Th- | 6384 | 0.083 | 2.505 | 2.834 |
| | KKK-εK-IL-6 (C73-C83) | 6433 | 0.061 | 5.174 | 4.894 | | KKK-εK-IL-6 (C73-C83) | 6385 | 0.080 | 5.337 | 5.201 |
| | | 6434 | 0.068 | 5.193 | 4.939 | | | 6386 | 0.084 | 3.830 | 4.881 |
| | | Avg | 0.064 | 5.167 | 4.917 | | | Avg | 0.082 | 3.891 | 4.305 |
| 16 | UBITh ®3-εK-KKK- | 6465 | 0.082 | 7.387 | 5.788 | 13 | KKKMvF3 Th-KKK-εK- | 6411 | 0.077 | 0.807 | 1.987 |
| | IL-6 (C73-C83) | 6466 | 0.096 | 5.458 | 5.214 | | IL-6 (C73-C83) | 6412 | 0.095 | 4.880 | 4.837 |
| | | 6467 | 0.111 | 6.062 | 5.385 | | | 6413 | 0.186 | 3.963 | 4.471 |
| | | Avg | 0.096 | 6.302 | 5.462 | | | Avg | 0.119 | 3.217 | 3.765 |
| 38 | Clostridium tetani TT4 Th- | 6444 | 0.115 | 5.395 | 5.292 | 43 | EBV CP Th-KKK-εK- | 6447 | 0.088 | 2.120 | 2.810 |
| | KKK-εK-IL-6 (C73-C83) | 6445 | 0.167 | 4.896 | 4.967 | | IL-6 (C73-C83) | 6448 | 0.068 | 1.101 | 2.177 |
| | | 6446 | 0.086 | 3.644 | 3.395 | | | 6449 | 0.074 | 3.623 | 3.975 |
| | | Avg | 0.123 | 4.645 | 4.551 | | | Avg | 0.077 | 2.281 | 2.987 |
| 28 | UBITh ®2-εK-KKK- | 6462 | 0.094 | 11.29 | >10 | 33 | Cholera Toxin Th-KKK-εK- | 6405 | 0.143 | 0.000 | 0.000 |
| | IL-6 (C73-C83) | 6463 | 0.143 | 4.215 | 4.754 | | IL-6 (C73-C83) | 6406 | 0.084 | 2.360 | 3.649 |
| | | 6464 | 0.095 | 4.553 | 4.984 | | | 6407 | 0.083 | 4.848 | 4.840 |
| | | Avg | 0.111 | 6.685 | 7.246 | | | Avg | 0.103 | 2.403 | 2.830 |
| 45 | EBV BPLF1 Th-KKK-εK- | 6456 | 0.083 | 2.948 | 3.035 | 52 | Schistosoma mansoni Th- | 6402 | 0.070 | 2.533 | 3.341 |
| | IL-6 (C73-C83) | 6457 | 0.084 | 3.552 | 4.506 | | KKK-εK-IL-6 (C73-C83) | 6403 | 0.084 | 3.444 | 3.452 |
| | | 6458 | 0.078 | 2.525 | 2.397 | | | 6404 | 0.087 | 0.000 | 0.374 |
| | | Avg | 0.082 | 3.008 | 3.313 | | | Avg | 0.081 | 1.992 | 2.389 |

1. A promiscuous artificial T helper cell (Th) epitope selected from the group consisting of SEQ ID NOs: 32-52.

2. A peptide immunogen construct represented by the following formulae:
(A)ₙ-(Target antigenic site)-(B)ₒ-(Th)ₘ-(A)ₙ-X
or
(A)ₙ-(Th)ₘ-(B)ₒ-(Target antigenic site)-(A)ₙ-X
or
(A)ₙ-(Th)ₘ-(B)ₒ-(Target antigenic site)-(B)ₒ-(Th)ₘ-(A)ₙ-X
or
{(A)ₙ-(Th)p-(B)ₒ-(Target antigenic site)-(B)ₒ-(Th)ₚ-(A)ₙ-X}ₘ
wherein:
each A is independently an amino acid;
each B is independently a heterologous spacer;
each Th is independently a promiscuous artificial Th epitope selected from the group consisting of SEQ ID NOs: 1-52;
the Target antigenic site is a B cell epitope from a foreign-antigen protein, a self-antigen protein, a CTL epitope, a Tumor-Associated Carbohydrate Antigen (TACA), a B cell epitope from a neoantigen, a small molecule drug, or an immunologically reactive analogue thereof;
X is an amino acid, α-COOH, or α-CONH₂;
n is 0, 1, 2, 3, 4, 5, 6, 7, 8, 9, or 10;
m is 1, 2, 3, or 4; and
o is 0, 1, 2, 3, 4, 5, 6, 7, 8, 9, or 10; and
p is 0, 1, 2, 3, or 4.

3. (canceled)

4. The peptide immunogen construct of claim 2, wherein the target antigenic site is a B cell epitope from a self-antigen protein selected from the group consisting of:
(a) an Aβ peptide having the amino acid sequence of SEQ ID NO: 56, 57, 58, 59, or 60;
(b) an alpha-Syn peptide having the amino acid sequence of SEQ ID NO: 61;
(c) an IgE EMPD peptide having the amino acid sequence of SEQ ID NO: 62;
(d) a Tau peptide having the amino acid sequence of SEQ ID NO: 63, 69, 70, or 71;
(e) an IL-31 peptide having the amino acid sequence of SEQ ID NO: 64 or 72; and
(f) an IL-6 peptide having the amino acid sequence of SEQ ID NO: 145.

5. The peptide immunogen construct of claim 2, wherein the heterologous spacer of component B is selected from the group consisting of an amino acid, Lys-, Gly-, Lys-Lys-Lys-, (α, ε-N)Lys, ε-N-Lys-Lys-Lys-Lys (SEQ ID NO: 53), Lys-Lys-Lys-εNLys (SEQ ID NO: 54), Gly-Gly, Pro-Pro-Xaa-Pro-Xaa-Pro (SEQ ID NO: 55), and any combination thereof.

6.-11. (canceled)

12. The peptide immunogen construct of claim 2, wherein the target antigenic site is a CTL epitope having an amino acid sequence selected from the group consisting of SEQ ID NOs: 76-144.

13.-17. (canceled)

18. The peptide immunogen construct of claim 2, wherein the target antigenic site is a TACA selected from the group consisting of GD3, GD2, Globo-H, GM2, Fucosyl GM1, GM2, PSA, Le^{y}, Le^{x}, SLe^{x}, SLe^{a}, Tn, TF, and STn.

19. The peptide immunogen construct of claim 2, wherein the target antigenic site is a B cell epitope from a neoantigen selected from the group consisting of SEQ ID NOs: 73-75.

20. The peptide immunogen construct of claim 2, wherein the target antigenic site is a small molecule drug.

21.-22. (canceled)

23. A pharmaceutical composition comprising the peptide immunogen construct according to claim 2.

24. A method of preventing and/or treating a disease, condition, or ailment in a subject comprising administering a pharmaceutically affective amount of the pharmaceutical composition of claim 23 to the subject.

25. The method according to claim 24, wherein the disease, condition, or ailment is HIV and wherein the target antigenic site is a CTL epitope from HIV selected from the group consisting of SEQ ID NOs: 76-82.

26. The method according to claim 24, wherein the disease, condition, or ailment is HSV and wherein the target antigenic site is a CTL epitope from HSV selected from the group consisting of SEQ ID NOs: 83-106.

27. The method according to claim 24, wherein the disease, condition, or ailment is FMDV and wherein the target antigenic site is a CTL epitope from FMDV selected from the group consisting of SEQ ID NOs: 107-123.

28. The method according to claim 24, wherein the disease, condition, or ailment is PRRSV and wherein the target antigenic site is a CTL epitope from PRRSV selected from the group consisting of SEQ ID NOs: 124-142.

29. The method according to claim 24, wherein the disease, condition, or ailment is CSFV and wherein the target antigenic site is a CTL epitope from CSFV selected from the group consisting of SEQ ID NOs: 143-144.

30. (canceled)

31. The method according to claim 24, wherein the disease, condition, or ailment is cancer and wherein the target antigenic site is a TACA selected from the group consisting of GD3, GD2, Globo-H, GM2, Fucosyl GM1, GM2, PSA, Ley, Lex, SLex, SLea, Tn, TF, and STn.

32. The method according to claim 24, wherein the disease, condition, or ailment is cancer and wherein the target antigenic site is a B cell epitope from a neoantigen selected from the group consisting of SEQ ID NOs: 73-75.

33. A method for tailoring an immune response in a subject comprising:
(a) preparing more than one peptide immunogen construct according to claim 2, wherein the Target antigenic site remains constant and the Th epitope is different on each peptide immunogen construct;
(b) preparing more than one pharmaceutical composition, each of which comprises one of the peptide immunogen constructs prepared in (a) and a pharmaceutically acceptable adjuvant or carrier;
(c) administering each of the pharmaceutical compositions prepared in (b) to different subjects;
(d) monitoring the immune response in each of the subjects; and
(e) selecting the pharmaceutical composition that produces a desired immune response.

34. The method according to claim 33, wherein the pharmaceutically acceptable adjuvant or carrier in each of the pharmaceutical compositions is the same.

35. The method according to claim 33, wherein the pharmaceutically acceptable adjuvant or carrier in each of the pharmaceutical compositions is different.
